SEQUENCE LISTING (23 sequences)

SEQ ID NO: 1 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
gggagagatgagagagaaatt

SEQ ID NO: 2 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
gtgtagtgctgtaaagaaatt

SEQ ID NO: 3 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
gcaaaatgtcgtcgaataatt

SEQ ID NO: 4 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
tggaaaaggtgtcgacaattt

SEQ ID NO: 5 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
ggacttcaggaacttcctgtt

SEQ ID NO: 6 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  modified_base 1: mod_base = OTHER; note = 5'Disulfide guanosine
  modified_base 9: mod_base = OTHER; note = 2'-methoxyuridine
  misc_feature 1..19: note = RNA
  misc_feature 20..21: note = DNA
gggagagatgagagagaaatt

SEQ ID NO: 7 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
cttaataactagaggagaatt

SEQ ID NO: 8 - DNA, 20 bp, synthetic construct
  source 1..20: mol_type = other DNA; note = Description of Artificial Sequence: Synthetic oligonucleotide; note = Description of Combined DNA/RNA Molecule: Synthetic oligonucleotide
  misc_feature 1..18: note = RNA
  misc_feature 19..20: note = DNA
cgcaaaggatacgaaatctt

SEQ ID NO: 9 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
ggataagcactacggcaaatt

SEQ ID NO: 10 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
tttctctctcatctctccctt

SEQ ID NO: 11 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  modified_base 1: mod_base = OTHER; note = Phosphate 2'-methoxyuridine
  modified_base 2: mod_base = OTHER; note = 2'-methoxyuridine
  modified_base 3: mod_base = OTHER; note = 2'-fluoro-deoxyuridine
  modified_base 5: mod_base = OTHER; note = 2'-fluoro-deoxyuridine
  modified_base 7: mod_base = OTHER; note = 2'-fluoro-deoxyuridine
  modified_base 14: mod_base = OTHER; note = 2'-fluoro-deoxyuridine
  modified_base 16: mod_base = OTHER; note = 2'-fluoro-deoxyuridine
  modified_base 19: mod_base = OTHER; note = Cytidine phosphorothioate
  modified_base 20: mod_base = OTHER; note = Deoxythymidine phosphorothioate
  modified_base 21: mod_base = OTHER; note = Deoxythymidine phosphorothioate
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..13: note = RNA
  misc_feature 14: note = DNA
  misc_feature 15: note = RNA
  misc_feature 16: note = DNA
  misc_feature 17..19: note = RNA
  misc_feature 20..21: note = DNA
tttctctctcatctctccctt

SEQ ID NO: 12 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
tttctttacagcactacactt

SEQ ID NO: 13 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
ttattcgacgacattttgctt

SEQ ID NO: 14 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
attgtcgacaccttttccatt

SEQ ID NO: 15 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  misc_feature 1..19: note = RNA
  misc_feature 20..21: note = DNA
caggaagttcctgaagtcctt

SEQ ID NO: 16 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  modified_base 1: mod_base = OTHER; note = Phosphate uridine
  misc_feature 1..19: note = RNA
  misc_feature 20..21: note = DNA
ttctcctctagttattaagtt

SEQ ID NO: 17 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  modified_base 1: mod_base = OTHER; note = Phosphate guanosine
  misc_feature 1..19: note = RNA
  misc_feature 20..21: note = DNA
gattccgtatcctttgccgtt

SEQ ID NO: 18 - DNA, 21 bp, synthetic construct
  source 1..21: mol_type = other DNA; note = Description of Artificial Sequence: Synthetic oligonucleotide; note = Description of Combined DNA/RNA Molecule: Synthetic oligonucleotide
  modified_base 1: mod_base = OTHER; note = Phosphate uridine
  misc_feature 1..19: note = RNA
  misc_feature 20..21: note = DNA
tttgccgtagtgcttatcctt

SEQ ID NO: 19 - protein, 201 aa, Homo sapiens
MNQNTTEPVAATETLAEVPEHVLRGLPEEVRLFPSAVDKTRIGVWATKPILKGKKFGPFVGDKKKRSQVKNNVYMWEVYYPNLGWMCIDATDPEKGNWLRYVNWACSGEEQNLFPLEINRAIYYKTLKPIAPGEELLVWYNGEDNPEIAAAIEEERASARSKRSSPKSRKGKKKSQENKNKGNKIQDIQLKTSEPDFTSAN

SEQ ID NO: 20 - protein, 1481 aa, Homo sapiens
MRDSAEGPKEDEEKPSASALEQPATLQEVASQEVPPELATPAPAWEPQPEPDERLEAAACEVNDLGEEEEEEEEEDEEEEEDDDDDELEDEGEEEASMPNENSVKEPEIRCDEKPEDLLEEPKTTSEETLEDCSEVTPAMQIPRTKEEANGDVFETFMFPCQHCERKFTTKQGLERHMHIHISTVNHAFKCKYCGKAFGTQINRRRHERRHEAGLKRKPSQTLQPSEDLADGKASGENVASKDDSSPPSLGPDCLIMNSEKASQDTINSSVVEENGEVKELHPCKYCKKVFGTHTNMRRHQRRVHERHLIPKGVRRKGGLEEPQPPAEQAQATQNVYVPSTEPEEEGEADDVYIMDISSNISENLNYYIDGKIQTNNNTSNCDVIEMESASADLYGINCLLTPVTVEITQNIKTTQVPVTEDLPKEPLGSTNSEAKKRRTASPPALPKIKAETDSDPMVPSCSLSLPLSISTTEAVSFHKEKSVYLSSKLKQLLQTQDKLTPAGISATEIAKLGPVCVSAPASMLPVTSSRFKRRTSSPPSSPQHSPALRDFGKPSDGKAAWTDAGLTSKKSKLESHSDSPAWSLSGRDERETVSPPCFDEYKMSKEWTASSAFSSVCNQQPLDLSSGVKQKAEGTGKTPVQWESVLDLSVHKKHCSDSEGKEFKESHSVQPTCSAVKKRKPTTCMLQKVLLNEYNGIDLPVENPADGTRSPSPCKSLEAQPDPDLGPGSGFPAPTVESTPDVCPSSPALQTPSLSSGQLPPLLIPTDPSSPPPCPPVLTVATPPPPLLPTVPLPAPSSSASPHPCPSPLSNATAQSPLPILSPTVSPSPSPIPPVEPLMSAASPGPPTLSSSSSSSSSSSSFSSSSSSSSPSPPPLSAISSVVSSGDNLEASLPMISFKQEELENEGLKPREEPQSAAEQDVVVQETFNKNFVCNVCESPFLSIKDLTKHLSIHAEEWPFKCEFCVQLFKDKTDLSEHRFLLHGVGNIFVCSVCKKEFAFLCNLQQHQRDLHPDKVCTHHEFESGTLRPQNFTDPSKAHVEHMQSLPEDPLETSKEEEELNDSSEELYTTIKIMASGIKTKDPDVRLGLNQHYPSFKPPPFQYHHRNPMGIGVTATNFTTHNIPQTFTTAIRCTKCGKGVDNMPELHKHILACASASDKKRYTPKKNPVPLKQTVQPKNGVVVLDNSGKNAFRRMGQPKRLNFSVELSKMSSNKLKLNALKKKNQLVQKAILQKNKSAKQKADLKNACESSSHICPYCNREFTYIGSLNKHAAFSCPKKPLSPPKKKVSHSSKKGGHSSPASSDKNSNSNHRRRTADAEIKMQSMQTPLGKTRARSSGPTQVPLPSSSFRSKQNVKFAASVKSKKPSSSSLRNSSPIRMAKITHVEGKKPKAVAKNHSAQLSSKTSRSLHVRVQKSKAVLQSKSTLASKKRTDRFNIKSRERSGGPVTRSLQLAAAADLSENKREDGSAKQELKDFRNFL

SEQ ID NO: 21 - DNA, 28 bp, synthetic construct
  source 1..28: mol_type = other DNA
ttggtggtggtggttgtggtggtggtgg

SEQ ID NO: 22 - DNA, 26 bp, synthetic construct
  source 1..26: mol_type = other DNA
ggtggtggtggttgtggtggtggtgg

SEQ ID NO: 23 - DNA, 12 bp, unidentified
  source 1..12: mol_type = unassigned DNA; note = C/T-rich consensus sequence
actttcactttc

(19) United States
(12) Patent Application Publication
(10) Pub. No.: US 2026/0224735 A1
(43) Pub. Date: Aug. 6, 2026

(54) TARGETED ANTI-PRDM COMPOSITIONS AND USES THEREOF

(71) Applicant: ARIZ PRECISION MEDICINE, INC., Davis, CA (US)

(72) Inventor: Brad J. NILES, Davis, CA (US)

(73) Assignee: ARIZ PRECISION MEDICINE, INC., Davis, CA (US)

(21) Appl. No.: 19/147,066

(22) PCT Filed: Jan. 10, 2024
(86) PCT No.: PCT/US2024/011064

§ 371 Date: Jul. 10, 2025

Related U.S. Application Data
(60) Provisional application No. 63/479,301, filed on Jan. 10, 2023.

(51) Int. Cl. A61K 47/69; A61K 9/51; A61K 45/06; A61K 47/54; A61P 35/00; C12N 15/113; C12N 15/115

(52) U.S. Cl. CPC A61K 47/6925; A61K 9/5115; A61K 45/06; A61K 47/549; A61P 35/00; C12N 15/1135; C12N 15/115; C12N 2310/11; C12N 2310/14; C12N 2310/16; C12N 2310/351

(57) ABSTRACT

Inhibitory RNA molecules and specifically targeted compositions that specifically inhibit mammalian PRDM expression, with therapeutic effect in cell proliferative diseases, such as cancer.

### CROSS REFERENCE TO RELATED APPLICATION

This application claims the benefit of U.S. Provisional Application No. 63/479,301, filed Jan. 10, 2023, and which is hereby incorporated by reference in its entirety.

### SEQUENCE LISTING

The instant application contains a Sequence Listing which has been filed electronically. The Sequence Listing is provided as a filed entitled “60673-707_601_SL.xml”, created Jan. 9, 2024, which is 56,430 bytes in size. The information in the electronic format of the Sequence Listing is incorporated by reference in its entirety.

### BACKGROUND

Small interfering RNAs (siRNAs) represent a new and emerging therapeutic area that has shown promising results in a number of diseases. Using siRNA it is possible to target a specific transcript for degradation, and therefore control the expression of a particular gene, whose expression is implicated in a disease. Cancer is a major health concern worldwide, being the second leading cause of death in the world. It is complex multifactorial disease, and understanding key biomolecular components that are associated with the disease etiology is crucial in order to generate candidates that can be regulated by novel therapeutic approaches, including, but not limited to siRNA. Additionally, specific targeting of siRNA to the disease site is crucial for limiting off-target effects and increasing efficiency and can be a deciding factor towards a successful therapeutic development.

### SUMMARY

In one aspect, provided herein is a pharmaceutical composition for treating a cancer in a subject in need thereof, comprising (i) a therapeutically effective amount of an inhibitor of a PRDM isoform and (ii) a cancer cell targeting moiety that binds specifically to a nuclear protein that is expressed on a cancer cell surface. In some embodiments, the PRDM isoform is a PRDM2 isoform, in particular, a RIZ2 isoform. In some embodiments, the PRDM inhibitor is a PRDM2-RIZ2 inhibitor, selected from a group consisting of small molecules, peptides, antibodies and nucleic acid inhibitors. In some embodiments, the PRDM inhibitor is an anti-PRDM-2 (RIZ2) siRNA. In some embodiments, the molar ratio of the inhibitor of a PRDM isoform to the targeting moiety is 1:10 to 10:1 or 1:100 to 100:1. In some embodiments, the molar ratio of the inhibitor of a PRDM isoform to the targeting moiety is 1:4 to 4:1.

In some embodiments, the pharmaceutical composition inhibits tumor cell growth in vivo as determined in a mouse xenograft tumor model. In some embodiments, the mouse xenograft tumor model is an NSCLC model. In some embodiments, the mouse is an athymic mouse. In some embodiments, the mouse is an immunocompromised mouse. Cells of the A549 lung carcinoma cell line is injected subcutaneously in the mouse to generate a tumor of the human lung cancer cells. Treatment of the siRNA on the tumor results in inhibition of the tumor growth. Accordingly, the pharmaceutical composition is determined to inhibit tumor cell growth in vivo. In some embodiments, the pharmaceutical composition is antiproliferative to cancer cells. In some embodiments, the pharmaceutical composition is tumor static. In some embodiments, the pharmaceutical composition is tumoricidal.

In some embodiments, the nuclear protein that is expressed on a cancer cell surface is nucleolin. In some embodiments, the nuclear protein that is over-expressed on a cancer cell surface is nucleolin.

In some embodiments, the cancer cell targeting moiety is a DNA aptamer. In some embodiments, the cancer cell targeting moiety binds to nucleolin on the surface of a cancer cell.

In some embodiments, the cancer cell targeting moiety comprises a nucleic acid having a sequence set forth in SEQ ID NO: 21 or a sequence that has less than 3 nucleotides differing from that of SEQ ID NO: 21.

Provided herein is a composition for treating a cancer in a subject, comprising (i) a therapeutically effective amount of a PRDM inhibitor and (ii) a cancer cell targeting moiety that binds specifically to a nuclear protein that is expressed on a cancer cell surface. In some embodiments, the PRDM is a PRDM2. In some embodiments, the PRDM2 is RIZ2. In some embodiments, the PRDM inhibitor is selected from a group consisting of small molecules, peptides, antibodies and nucleic acid inhibitors. In some embodiments, the PRDM inhibitor is a methyl transferase inhibitor. In some embodiments, the PRDM inhibitor is an anti-PRDM-2 (RIZ2) siRNA.

In some embodiments, the composition described above comprises anti-PRDM2 siRNA that comprises a sense strand and an antisense strand, selected from Table 2. In some embodiments, the cancer cell targeting moiety is a DNA aptamer. In some embodiments, the DNA aptamer binds to a nuclear protein expressed on the cancer cell surface.

In some embodiments, the nuclear protein expressed on the surface of a cancer cell is nucleolin.

In some embodiments, the DNA aptamer binds to nucleolin on the surface of a cancer cell.

In some embodiments, the cancer cell targeting moiety comprises a nucleic acid having a sequence with at least 85% sequence identity to the sequence of SEQ ID NO: 21.

In some embodiments, the cancer cell targeting moiety comprises a nucleic acid having a sequence with at least 90% sequence identity to the sequence of SEQ ID NO: 21.

In some embodiments, the cancer cell targeting moiety comprises a nucleic acid having a sequence with at least 95% sequence identity to the sequence of SEQ ID NO: 21.

In some embodiments, the cancer cell targeting moiety comprises a nucleic acid having a sequence set forth in SEQ ID NO: 21 or a sequence that has less than 3 nucleotides differing from that of SEQ ID NO: 21.

In some embodiments, the composition further comprises a nanoparticle delivery vehicle, comprising: (a) the therapeutically effective amount of an anti-PRDM2 siRNA comprising the sense strand and the antisense strand, wherein the sense strand and the antisense strand are selected from Table 2; (b) a DNA aptamer that binds to a nuclear protein expressed on the surface of a cancer cell having a sequence that is at least 85% sequence identity to the sequence of SEQ ID NO: 21.

In some embodiments, the nanoparticle delivery vehicle is a calcium phosphate nanoparticle delivery vehicle.

In some embodiments, the anti-PRDM2 siRNA molecule comprises a double stranded RNA comprising a sense strand and an antisense strand, each strand having 10-21 nucleotides, wherein the sense strand and the antisense strand hybridize with each other to form at least a region of double strands, and wherein each of the sense strand and the antisense strand has at least one nucleotide overhang at the 5′ or the 3′ end.

In some embodiments, the anti-PRDM2 siRNA molecule comprises at least 10 contiguous nucleotides that are homologous to 10 contiguous nucleotides of a human PRDM2/RIZ2 gene.

In some embodiments, the anti-PRDM2 siRNA molecule and the DNA aptamer are admixed together with the calcium nanoparticle.

In some embodiments, the anti-PRDM2 siRNA molecule is coupled to the DNA aptamer.

In some embodiments, the anti-PRDM2 siRNA molecule is coupled to the DNA aptamer non-covalently. In some embodiments, the anti-PRDM2 siRNA molecule and the DNA aptamer are both bound to a nanoparticle described herein. In some embodiments, the anti-PRDM2 siRNA molecule is coupled to the DNA aptamer covalently.

In some embodiments, a composition described herein comprises one or more anti-PRDM2 siRNA molecules. In some embodiments, the anti-PRDM2 siRNA molecule is covalently linked to the DNA aptamer. In some embodiments, the anti-PRDM2 siRNA molecule is associated with a poly-ethylene glycol (PEG) moiety. In some embodiments, the anti-PRDM2 siRNA molecule is covalently linked with a poly-ethylene glycol (PEG) moiety (pegylated siRNA). In some embodiments, at least 5 mol % the anti-PRDM2 siRNA molecules (e.g., ARIZ-047) is pegylated. In some embodiments, at least 2 mol %, at least 5 mol %, at least 8 mol %, at least 10 mol %, at least 20 mol %, or at least 50 mol % of the anti-PRDM2 siRNA molecules are pegylated. In some embodiments, 5-20 mol %, 2-30 mol %, 8-15 mol %, 10-50 mol %, 1-10 mol %, 50-99 mol %, or 20-80 mol % of the anti-PRDM2 siRNA molecules are pegylated. In some embodiments, 10-15 mol % of the anti-PRDM2 siRNA molecules are pegylated. In some embodiments, about 12.5 mol % of the anti-PRDM2 siRNA molecules are pegylated. In some embodiments, a composition described herein comprises one or more DNA aptamers. In some embodiments, the DNA aptamer is pegylated. In some embodiments, at least 20 mol % the DNA aptamers are pegylated. In some embodiments, at least 10 mol %, at least 25 mol %, at least 50 mol %, at least 75 mol %, at least 90 mol %, at least 95 mol %, or at least 99 mol % of the DNA aptamers are pegylated. In some embodiments, 10-99 mol %, 20-95 mol %, 50-95 mol %, 60-95 mol %, or 50-80 mol % of the DNA aptamers are pegylated. In some embodiments, 70-80 mol % the DNA aptamers are pegylated. In some embodiments, about 75 mol % the DNA aptamers are pegylated.

In some embodiments, the DNA aptamer is exposed on the surface of the nanoparticle delivery vehicle.

Provided herein is a pharmaceutical composition comprising the composition of any one of embodiments described above, and a pharmaceutically acceptable excipient. In some embodiments, the anti-PRDM2 siRNA molecule comprises a sense strand having a sequence that is at least 95% identical to SEQ ID NO: 6, and an antisense strand having a sequence that is at least 95% identical to SEQ ID NO: 11. In some embodiments, the the anti-PRDM2 siRNA molecule comprises a sense strand having a sequence set forth in SEQ ID NO: 6, and an antisense strand having a sequence set forth in SEQ ID NO: 11. In some embodiments, the anti-PRDM2 siRNA molecule inhibits human PRDM2/RIZ2 expression.

In some embodiments, the anti-PRDM2 siRNA molecule inhibits a tumor growth.

In some embodiments, the anti-PRDM2 siRNA molecule comprises a sense strand sequence that is at least 80% identical to the sequence set forth in SEQ ID NO: 1, and an antisense strand sequence that is at least 80% identical to the sequence set forth in SEQ ID NO: 10. In some embodiments, the anti-PRDM2 siRNA molecule comprises a sense strand sequence that is at least 90% identical to the sequence set forth in SEQ ID NO: 1, and an antisense sequence that is at least 90% identical to the sequence set forth in SEQ ID NO: 10.

In some embodiments, the anti-PRDM2 siRNA molecule comprises a sequence that is at least 95% identical to the sequence set forth in SEQ ID NO: 1, and a sequence that is at least 95% identical to the sequence set forth in SEQ ID NO: 10.

In some embodiments, the anti-PRDM2 siRNA molecule comprises one or more modified ribonucleotide bases.

In some embodiments, the anti-PRDM2 siRNA molecule comprises a pseudouridine, a 5-methylcytosine, or both.

In some embodiments, the anti-PRDM2 siRNA molecule comprises one or more of: a deoxythymidine (dTdT) modification at the 3′-terminus or the 5′ terminus, a phosphorothioate linkage between two consecutive nucleotide bases and a 5′-phosphate (5′-P) on the sense strand or the antisense strand.

In some embodiments, the anti-PRDM2 siRNA molecule comprises a 5′ terminal phosphate group on the sense strand or the anti-sense strand.

In some embodiments, the anti-PRDM2 siRNA molecule comprises not more than 3 nucleotides mismatch between the sense and the antisense strand.

In some embodiments, the anti-PRDM2 siRNA molecule is encapsulated in the calcium phosphate nanoparticle delivery vehicle.

In some embodiments, the nanoparticles have a particle size ranging from 10 nm-1 mm in diameter.

In some embodiments, the nanoparticle comprises a Ca:P molar ratio that ranges between 0.9-1.67. In some embodiments, the Ca:P molar ratio is between 1 to 1.2.

In some embodiments, the aptamer forms G-quadruplex structures.

In some embodiments, the molar ratio of the anti-PRDM2 siRNA molecule to the cell targeting moiety is from about 50:1 to about 1:50, about 10:1 to about 1:10, about 5:1 to about 1:5, about 2:1 to about 1:2, or about 1.5:1 to about 1:1.5.

In some embodiments, the molar ratio of the anti-PRDM2 siRNA molecule to the cell targeting moiety is about 1:1.

In some embodiments, the delivery vehicle comprises one or more polyethylene glycols, and wherein the polyethylene glycols comprise amine, carboxy, sulfahydryl, phosphate, maleimide or other active groups bonded to the PEG.

In some embodiments, an average molecular weight of the polyethylene glycols ranges from 500 Da to 2000 Da.

In some embodiments, the polyethylene glycols are bonded to the siRNA via a phosphoamide, amide, disulfide, thioester or other chemical bond.

In some embodiments, the therapeutically effective amount of the inhibitory RNA is 10 ng-100 milligrams.

In some embodiments, the pharmaceutically acceptable excipient is an aqueous solution.

In some embodiments, the composition is formulated as an injectable solution.

In some embodiments, the composition is formulated for pulmonary delivery.

In some embodiments, the composition is formulated for nebulization.

Provided herein is a pharmaceutical composition comprising a calcium phosphate nanoparticle delivery vehicle, comprising: (a) a therapeutically effective amount of an anti-PRDM2 siRNA comprising a sense strand having a sequence set forth in SEQ ID NO: 6; and an anti-sense strand having a sequence set forth in SEQ ID NO: 11 (b) a cancer cell targeting DNA aptamer, having a sequence of SEQ ID NO: 21, wherein the cancer cell targeting DNA aptamer binds to nucleolin on a cancer cell.

Provided herein is an injectable formulation for anti-cancer therapy, comprising:
(a) a double stranded siRNA having a sense strand comprising a sequence of SEQ ID NO: 6; and an antisense comprising a sequence of SEQ ID NO: 11;
(b) a calcium phosphate nanoparticle encapsulating the siRNA of (a);
(c) a cancer cell targeting moiety covalently associated with (a) or (b), having a nucleic acid sequence of SEQ ID NO: 21; and
(d) a pharmaceutically acceptable excipient.

In some embodiments, the composition comprises a calcium phosphate nanoparticle.

In some embodiments, the composition shows at least 1.1 fold higher tumoricidal activity than (i) AS1411 alone, or (ii) a comparable amount of the inhibitory RNA molecule within the calcium phosphate nanoparticle in absence of the aptamer. In some embodiments, the tumoricidal activity of at least 1.1 fold is at least 1.5 fold or at least 2 fold.

In some embodiments, a molar ratio of the inhibitory RNA molecule to the targeting agent in the pharmaceutical composition ranges from about 50:1 to about 1:50, about 10:1 to about 1:10, about 5:1 to about 1:5, about 2:1 to about 1:2, or about 1.5:1 to about 1:1.5.

In some embodiments, the molar ratio of the inhibitory RNA molecule (e.g., ARIZ-047) to the targeting agent (e.g., an aptamer such as As1411) is about 1:1. In some embodiments, the molar ratio of the inhibitory RNA molecule to the targeting agent is about 8:1. In some embodiments, the molar ratio of the inhibitory RNA molecule to the targeting agent is about 12:1 to 4:1.

In some embodiments, the delivery vehicle comprises one or more polyethylene glycols, and wherein the polyethylene glycols comprise amine, carboxy, sulfahydryl, phosphate, maleimide or other groups bonded to the PEG.

In some embodiments, the polyethylene glycols range in size from 500 Da to 20 Da. In some embodiments, the polyethylene glycol (PEG) chain has a molecular weight of 2000. In some embodiments, the PEG has a molecular weight of about 400-5000 Da. In some embodiments, the PEG has a molecular weight of about 1000-3000 Da. In some embodiments, the PEG has a molecular weight of about 400-10,000 Da. In some embodiments the PEG is bifunctional, e.g., having two reactive ends suitable for bi-conjugation or crosslinking two entities. In some embodiments the PEG is homobifunctional, e.g., having two reactive ends that are the same. In some embodiments, the PEG is heterobifunctional, e.g., having two reactive ends that are non-identical, with different binding properties. In some embodiments, the PEG is linear, in some embodiments, the PEG has multiple reactive arms. In some embodiments, the PEG can be Y-shaped. In some embodiments, about 12.5% of the siRNA molecules are pegylated.

In some embodiments, the polyethylene glycols are bonded to the siRNA via a phosphoamide, amide, disulfide, thioester or other chemical bond.

In some embodiments of the pharmaceutical composition, the therapeutically effective amount of the inhibitory RNA is 10 ng-100 milligrams per dose.

The pharmaceutical composition of claim 1, wherein the therapeutically effective amount of the inhibitory RNA is 1-50 micrograms. In some embodiments, the pharmaceutically acceptable excipient is an aqueous solution. In some embodiments, the composition is formulated as an injectable solution.

In some embodiments, a single dose of the injectable solution is 0.1-2 ml. In some embodiments, the composition is formulated for pulmonary delivery. In some embodiments, the composition is formulated for nebulization.

Provided herein is a pharmaceutical composition, comprising (i) a calcium phosphate nanoparticle, wherein the nanoparticle comprises (a) a pegylated siRNA, wherein the pegylated siRNA has a sequence that is at least 90% identical to any one of the sequences set forth in Table 2, wherein the pegylated siRNA comprises 10-2000 ethylene glycol units, and (b) a pegylated aptamer, wherein the aptamer has a sequence that is at least 90% identical to the sequence set forth in SEQ ID NO: 21, wherein the pegylated aptamer comprises 10-2000 ethylene glycol units, wherein a molar ratio of the pegylated siRNA to the pegylated aptamer is about 10:1 to 1:10; and (ii) a pharmaceutically acceptable excipient.

In one aspect, provided herein is an injectable formulation for anti-cancer therapy, comprising: (a) 1-20 micrograms of an siRNA having any one of the sequences of Table 2 (e.g., a sense strand of SEQ ID NO: 6 and an antisense strand of SEQ ID NO: 11); (b) a calcium phosphate nanoparticle encapsulating the siRNA of (a); (c) a targeting agent covalently associated with (a) or (b), wherein the targeting is an anti-nucleolin aptamer AS1411; and (d) a pharmaceutically acceptable excipient.

In some embodiments of the composition described herein, the cancer is selected from lung cancer, liver cancer, pancreatic cancer, breast cancer, bladder cancer, prostate cancer, colon and rectal cancer, kidney cancer, endometrial cancer, head and neck cancer, thyroid cancer, melanoma and leukemia.

Provided herein is a method of treating a cancer in a subject in need thereof comprising, administering to the subject a pharmaceutical composition described herein.

In some embodiments, the pharmaceutical composition comprises an injectable formulation comprising at least 1-100 microgram of the inhibitory RNA molecule that comprises a sense strand having is at least 80% identical to a sequence set forth in SEQ ID NO: 1, and an antisense strand having is at least 80% identical to a sequence set forth in SEQ ID NO: 10. In some embodiments, the pharmaceutical composition comprises an injectable formulation comprising at least 1-100 microgram of the inhibitory RNA molecule that comprises a sense strand having is at least 90% identical to a sequence set forth in SEQ ID NO: 1, and an antisense strand having is at least 90% identical to a sequence set forth in SEQ ID NO: 10. In some embodiments, the pharmaceutical composition comprises an injectable formulation comprising at least 1-100 microgram of the inhibitory RNA molecule that comprises a sense strand having is at least 95% identical to a sequence set forth in SEQ ID NO: 1, and an antisense strand having is at least 95% identical to a sequence set forth in SEQ ID NO: 10. In some embodiments, the pharmaceutical composition comprises an injectable formulation comprising at least 1-100 microgram of the inhibitory RNA molecule that comprises a sense strand having is at least 80% identical to a sequence set forth in SEQ ID NO: 6, and an antisense strand having is at least 80% identical to a sequence set forth in SEQ ID NO: 11. In some embodiments, the pharmaceutical composition comprises an injectable formulation comprising at least 1-100 microgram of the inhibitory RNA molecule that comprises a sense strand having is at least 90% identical to a sequence set forth in SEQ ID NO: 6, and an antisense strand having is at least 90% identical to a sequence set forth in SEQ ID NO: 11. In some embodiments, the pharmaceutical composition comprises an injectable formulation comprising at least 1-100 microgram of the inhibitory RNA molecule that comprises a sense strand having is at least 95% identical to a sequence set forth in SEQ ID NO: 6, and an antisense strand having is at least 95% identical to a sequence set forth in SEQ ID NO: 11. In some embodiments, the administration is via systemic, intravenous, intramuscular, intratumoral, or intrapulmonary delivery.

In some embodiments, the cancer is lung cancer.

Provided herein is a method of treating lung cancer in a human subject, comprising, administering to the subject a pharmaceutical composition comprising: (a) a therapeutically effective amount of an siRNA having the sense strand sequence of SEQ ID NO: 1 and the antisense strand sequence of SEQ IDN O: 10; (b) a calcium phosphate nanoparticle encapsulating the siRNA of (a); (c) a targeting agent associated with (a) or (b), wherein the targeting is an anti-nucleolin aptamer AS1411; and (d) a pharmaceutically acceptable excipient. In some embodiments, the targeting agent is covalently associated with (a) or (b). In some embodiments, the targeting agent is covalently associated with (a). In some embodiments, the targeting agent is covalently associated with (b).

In some embodiments, administering the composition leads to suppression of tumor growth by greater than 50% compared to vehicle control. In some embodiments, administering the composition is more effective at suppression of tumor growth as compared to an otherwise identical composition without the targeting agent. In some embodiments, the composition is more effective at suppression of tumor growth as compared to carboplatin. In some embodiments, the composition is more effective at suppression of tumor growth as compared to an otherwise identical composition without the targeting agent as determined by tumor volume in a xenograft model. In some embodiments, the tumor growth in the xenograft model that is treated with compositions of this disclosure is less than 50% compared to the tumor growth in the xenograft model that is treated with an otherwise identical composition without the targeting agent. In some embodiments, the tumor growth in the xenograft model that is treated with compositions of this disclosure is less than 80%, 60%, 50%, 40%, 30%, 20%, or 10% compared to the tumor growth in the xenograft model that is treated with an otherwise identical composition without the targeting agent.

Additional features of any of the aforesaid multifunctional molecules, nucleic acids, vectors, host cells, or methods include one or more of the following enumerated embodiments.

Those skilled in the art will recognize or be able to ascertain using no more than routine experimentation, many equivalents to the specific embodiments of the invention described herein. Such equivalents are intended to be encompassed by the following enumerated embodiments.

Additional features of any of the aforesaid multifunctional molecules, nucleic acids, vectors, host cells, or methods include one or more of the following enumerated embodiments.

### BRIEF DESCRIPTION OF THE FIGURES

FIG. 1A depicts graphical illustrations of two exemplary embodiments among the various embodiments of the invention described herein, comprising a drug delivery system containing an siRNA payload with complementarity to a PRDM mRNA. In one example shown on the left, the drug delivery system is a nanoparticle composition that encapsulates an siRNA, the siRNA is directly conjugated to an aptamer cell targeting element via a linker to specifically target the payload to cancer cells or cancer stem cells. In the examples shown on the right, the drug delivery system is a nanoparticle composition that encapsulates an siRNA, the siRNA is conjugated PEG conjugated; and the nanoparticle composition further comprises PEG-conjugated aptamer. The nanoparticle compositions in either example may further comprise an additional drug (not shown in figure) as an additional payload. Such additional payload(s) may be currently approved chemotherapy agents, repurposed drugs, or any drug that effectively aids in killing, or controlling the growth or spread of cancer cells. Individual components of the nanoparticle composition in the diagram are displayed and marked in the index within the box at the bottom.

FIG. 1B depicts data showing size information for the calcium phosphate nanoparticle after formation, as measured by dynamic light scattering.

FIG. 1C depicts data showing efficacy of the exemplary anti-RIZ2 siRNA described herein (comprising SEQ ID NO: 6 and SEQ ID NO: 11) in tumor cell killing in mouse-A549 xenograft model. A non-targeted siRNA control was used in which the same siRNA in absence of the targeting aptamer is present in the nanoparticle composition. For targeted nanoparticle composition comprising the exemplary siRNA, siRNA was conjugated with PEG, and mixed with PEG conjugated aptamer targeting nucleolin, and mixed with ingredients necessary for the nanoparticle composition coating under conditions that will form the nanoparticle composition. The nucleolin binding aptamer remains exposed on the surface of the nanoparticle composition, and targets the nanoparticle composition to a cancel cell which expresses nucleolin on the surface. Carboplatin is shown as a positive control. Vehicle control is 5% dextrose in water.

FIG. 1D depicts data showing downregulation of RIZ2 expression with siRNA formulated in a nanoparticle comprising the targeting moiety (AS1411) or without the targeting moiety.

FIG. 2 depicts data from RNA-Seq quantitative gene expression analysis showing effect of the exemplary anti-RIZ2 (PRDM2) siRNA (the exemplary anti-RIZ2 siRNA described herein, comprising SEQ ID NO: 6 and SEQ ID NO: 11) on A549 cells. The gene expression analysis was arranged in a volcano plot, with decreased expression on the left, and increased expression on the right (left panel), and arranged in gene ontology subgroups (right panel), each plot showing that distinct set of genes are differentially expressed. A total of 61 and 50 differentially expressed genes were down and upregulated, respectively. These results suggest that ARIZ047 treatment elicits significant changes in gene expression in A549 cells, providing insight into the molecular mechanisms underlying its therapeutic effects in lung cancer.

FIG. 3 depicts data from RNA-Seq analysis, showing the exemplary anti-RIZ2 siRNA-mediated change in RNA landscape enriched in gene ontology associated with anti-cancer function, in pathways that regulate cell proliferation, cell migration, and cell death. The exemplary anti-RIZ2 siRNA described herein comprises SEQ ID NO: 6 and SEQ ID NO: 11.

FIG. 4 depicts data demonstrating that weighted co-expression network (Bayesian) and unweighted network (non-Bayesian) both identified EGR1 as driver gene. FIG. 4A shows a gene dendrogram, obtained by clustering gene expression. The plot shows differentially expressed genes can be clustered into nine gene modules. FIG. 4B shows scale independence (top panel) and Mean connectivity (bottom panel) of the expression data, which guided the analysis shown in FIG. 4A. FIG. 4C Venn diagrams showing overlap of gene clusters leading to identification of EGR1 related genes as the driver gene family upregulated and mediating the anti-RIZ2 siRNA mediated antitumor activity; FIG. 4D shows a comparison of mRNA expression levels of Jun and EGR1, relative comparing the exemplary anti-RIZ2 siRNA (as described herein, comprising SEQ ID NO: 6 and SEQ ID NO: 11) treated or scRNA (scrambled siRNA) treated cells. The y-axis shows the fold increase in mRNA expression levels for anti-RIZ2 siRNA treated cells compared to scRNA treated cells. The data taken together shows EGR1 plays an important role in negative regulation of WNT signaling, insulin signaling, and Type1 interferon signaling.

FIG. 5A depicts data demonstrating that tumor cell killing by anti-RIZ2-siRNA could be blocked by treatment with R-espondin, an agonist of WNT pathway signaling.

FIG. 5B shows HEK-293 cells stably transfected with GFP or RIZ2-GFP and treated with the indicated Wnt-signaling agonists.

FIG. 5C shows bar graphs representing the relative fold change in RIZ2 lacking- and overexpressing-spheroid size (area) in the presence of Wnt-signaling agonists, which was calculated using the Application Suite Software (****p<0.0001, ***p=0.0005, **p<0.005, *p<0.05).

FIG. 6 depicts data demonstrating increase in PD-L1 expression in lung cancer cells upon treatment with an exemplary siRNA. The siRNA has a sense strand of SEQ ID NO: 6 and an antisense strand of SEQ ID NO: 11. A549 cells were treated with the siRNA, or treated with the scrambled (scr) control sequence or left untreated. siRNA treated cells exhibited about 6-fold increase in PD-L1 expression compared to either of the controls (untreated and scr).

The accompanying drawings numbered herein are given by way of illustration only and are not intended to be limitative to any extent.

### DETAILED DESCRIPTION

Current anti-cancer drug therapies are of limited effectiveness. Commonly used anti-cancer drugs may bring about temporary remission of tumors, and help prolong the patient's life, but in most cases are not curative because they often do not eliminate the cancer completely and tumors may subsequently reemerge. Small molecule chemotherapies are currently the most widely used treatment for cancer. However, in most therapeutic approaches, targeted and long-term efficacy is yet to be achieved.

Biologic and chemotherapeutic agents can have serious off-target toxic effects, causing collateral damage to normal, healthy cells and tissues as well as the cancerous cells that they are intended to control and destroy. Consequently, patients suffer from severe side effects due to toxicity. Immune based therapies were expected to decrease toxicity and improve survival, but despite their promise they have only incrementally improved the prospects for cancer treatment and for favorable long-term patient outcomes. Although targeted therapy and immunotherapy are increasingly used as supplements or alternatives to traditional chemotherapy, such therapies generally lack long-term effectiveness, as cancers commonly adapt and rapidly develop resistance, escaping the targeted therapy effect. For the foregoing reasons, chemotherapy, immunotherapy, and targeted biologic therapies are yet to deliver the promises of being curative. The need for specific, targeted and effective therapy remains unmet, and in many cases a combination of therapeutic modalities may prove to be the clinically effective approach.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. It is understood that terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. Nothing herein is intended as a promise.

The term “about” as used herein refers to the usual error range for the respective value readily known to the skilled person in this technical field. Reference to “about” a value or parameter herein includes (and describes) embodiments that are directed to that value or parameter per se. “About” can mean within ±10% of a value. For example, if it is stated, “a marker may be increased by about 50%,” it is implied that the marker may be increased between 45%-55%.

By “agent” is meant any small molecule chemical compound, antibody, nucleic acid molecule, or polypeptide, or fragments thereof.

The terms “polynucleotide” and “nucleic acid” are used interchangeably herein and refer to polymers of nucleotides of any length, and include DNA and RNA, for example, mRNA. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered using the methods of the invention is mRNA.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection.

Cell proliferative disorder or hyperproliferative disorder, may be considered as a disease or a disorder associated with an increased, in most cases uncontrolled cell proliferation due to disfunction of one or more cell cycle related genes. Exemplary hyperproliferative disorder may be a neoplasia, or a cancer. Cell cycle is the cyclic interval between two consecutive G0/G1 phases (or two consecutive G1/M phases) in the life cycle of a cell, which usually remains constant for a cell type under optimal environmental conditions. A cell cycle disorder may be a disorder that disrupts the normal cyclic interval between two consecutive G0/G1 phases (or two consecutive G1/M phases) in the life cycle of the cell, in general that may be caused by disruption of a cell cycle regulator. For example, an exemplary cell cycle regulator is cyclin D. In some embodiments, RIZ1 and/or RIZ2 may be considered regulators of cell cycle. In some embodiments, RIZ1 and/or RIZ2 may be considered master regulators of cell cycle, in that they control life cycle ubiquitously; or that they control a nodal point in the life cycle of a cell which is irrespective of the cell type, location or environmental conditions. In some embodiments, a tumoricidal agent described herein may reduce the proliferative rate of a cancer. In some embodiments, the tumoricidal agent may have an anti-proliferative action to a cancer. In some cases the tumoricidal and anti-proliferative functions may be interchangeably used.

Expression or gene expression may generally refer to the transcription process of a gene, resulting in mRNA product. The mRNA product may usually be further translated leading to protein generation. Gene expression may ultimately lead to the generation of the protein or the polypeptide product of transcription followed by translation of the gene. Expression on a cell surface may usually be considered to refer to a protein product that is present on a cell surface, or a biomolecule that is present on the cell surface. For example, nucleolin as a protein product is a nuclear protein in a normal healthy cell, e.g., a non-cancer cell. An aberrant expression of the protein nucleolin is observed in cancer, where nucleolin is expressed on the cell surface, e.g. the nucleolin protein is present exposed on the cell surface. In some embodiments, nucleolin is aberrantly expressed on the surface of a cancer cell. In some embodiments, nucleolin is overexpressed in a cancer cell, and is accumulated on a cell surface. In some embodiments, it can be understood that the molecule, e.g. nucleolin is predominantly expressed on the cell surface of a cancer cell. In some embodiments, it may be considered that nucleolin is not predominantly expressed on the surface of a non-cancer cell, e.g., a healthy cell, e.g., a healthy non-cancer cell, even though it is expressed in the nucleus of a healthy/non-cancer cell. In some embodiments, expression of the nucleolin on the exposed cell surface makes it an attractive target for selecting or targeting a cancer cell appreciably in preference to a non-cancer cell, which does not present exposed nucleolin on the cell surface.

Hyperplasia may be defined as a condition of rapid cell proliferation, e.g., as a result of disruption of a cell cycle regulator, e.g., a checkpoint inhibitor. Neoplasia is a condition where a cell has undergone a neoplastic transformation, e.g., a cancerous transformation.

RNA interference may refer to the process of sequence-specific post-transcriptional gene silencing in animals mediated by short interfering RNAs (siRNAs) (Zamore et al, 2000, Cell, 101, 25-33; Fire et al., 1998, Nature, 391, 806; Hamilton et al., 1999, Science, 286, 950-951; Lin et al., 1999, Nature, 402, 128-129; and Strauss, 1999, Science, 286, 886). The corresponding process in plants (Heifetz et al., International PCT Publication No. WO 99/61631) is commonly referred to as post-transcriptional gene silencing or RNA silencing and is also referred to as quelling in fungi. The presence of long dsRNAs in cells stimulates the activity of a ribonuclease III enzyme referred to as dicer (Bass, 2000, Cell, 101, 235; Zamore et al, 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

In some aspects, a reference, or a control may be used to represent a baseline or a steady state of a condition, e.g., an amount or a level of a transcript or a translated product that signifies a state in a non-diseased (e.g., non-cancer) cell, cell population or tissue or organism, such that with respect to said amount or level in the reference or control, the corresponding state in another exemplary set may be considered to be at a diseased or abnormal state (e.g., cancer). A reference or a control may often be as commonly used and may be well understood, based on the context, by one of skill in the art. In some embodiments, for example, the protein level of RIZ2 is higher in a cancer cell relative to a non-cancer cell, and the average level of the protein in a non-cancer cell, if known, could be used as a control or a reference. Similarly, the ratio of level of RIZ1 over RIZ2 in a cell (that is not a cancer cell and may otherwise be identical to a cancer cell excepting the diseased state, e.g., tissue or origin or from another human being that is not suffering from a cancer), could be used as a control or reference to determine whether the ratio of RIZ1 over RIZ2 levels in a cancer cell is a deviation from the reference level, in which, the said level in the control or reference is perceived as “normal” and the deviated level in the cancer cell as a deviation from the normal.

In some embodiments, when comparing gene expression levels between two states, e.g., before and after contracting a disease, or before and after a certain drug treatment, expression of a gene may be considered to be upregulated or downregulated. These terms may be understood to be relative to the former state, if the expression level has gone up or down respectively.

In some embodiments, the term nanoparticle is used to refer to a delivery vehicle for a drug or a biomolecule, wherein the nanoparticle is a encapsulating agent that harbors the drug or the biomolecule and is capable of transferring the drug or biomolecule across a cell membrane and deliver it to the inside of a cell. Nanoparticles may be limited in size, e.g., less than 1 micron, less than 500 nm, etc. Nanoparticles may have various compositions, as is exemplified to some extent elsewhere in the disclosure.

Targeting moiety may refer to a protein, peptide, DNA or any other biomolecule, or a chemical ligand that targets a certain product (e.g., a drug cargo) to a cell where the drug is expected to act, which may be referred to as a target cell. In some cases, for example, a cancer cell is a target for a drug, and a targeting moiety may be attached to the drug, such that the drug is subjected to specifically make a direct or indirect contact with the target cell, and the target cell eventually uptakes the drug.

Conjugation may refer to chemical process which requires a covalent bond formation between two conjugating elements, e.g., A and B. Fusion may refer to a chemical process where an attachment, physical or chemical may result between two fusing elements, e.g., A and B.

The term “inhibition” may be used often interchangeably with “reduction” or “reduction in expression levels” and may indicate reduction in the level of a particular component that is inhibited, wherein the reduction may be partial, (e.g., 20%, 50%, 75%, 80%), or complete (about 100%). For example, a RIZ2 inhibitor inhibits RIZ2 protein in a cell by about 50%, or about 100%.

A target cell may be considered a cell to which a pharmaceutical composition is directed. A target molecule or a target protein may be a protein that is present on a target cell, to which a pharmaceutical compound may be specifically directed. For example, a target cell may be a cancer cell, to which the pharmaceutical composition described herein is directed, e.g., via a targeting moiety to the target cell; wherein the targeting moiety in turn helps the association or proximation of the pharmaceutical compound in the pharmaceutical composition to the target cell. For example, as described herein, the pharmaceutical composition for cancer may comprise an siRNA and a targeting moiety, wherein the targeting moiety can be an aptamer: wherein the aptamer, by being capable of binding to a cell surface protein (target protein) on a cancer cell brings the pharmaceutical compound, e.g., the siRNA within the pharmaceutical composition in proximity to the cancer cell. For example, the target protein may be a protein overexpressed on a cancer cell. In some embodiments, the target protein may be a protein that is aberrantly expressed on a cancer cell surface, e.g., nucleolin. In some embodiments, the targeting moiety is structurally associated with the pharmaceutical compound. In some embodiments, the targeting moiety is covalently associated with the pharmaceutical compound. In some embodiments, the targeting moiety is not covalently associated with the pharmaceutical compound.

Any ranges, for example for concentrations, dimensions, weight, mass, volume, density measurements, percentages, or fractions that are described or presented herein are inferred to be inclusive of the terminal numbers, digits of the range disclosed. Where size is indicated in length, it may be understood that the indicated length is the largest diameter if denoting a spherical or semi-spherical object. For example, a nanoparticle size is designated by its diameter, which may be denoted as 1 micrometer, or by a range of 1 micrometer to 10 micrometer, where the nanoparticle exhibits a diameter to be 1, 2, 3, 4, 5, 6, 7, 8, 9, or 10 micrometers.

The terms “treatment,” “treating,” “alleviation” and the like, may be used in the context of a disease, injury or disorder, are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein may cover any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

Cancer is a multifactorial disease. The causative factors are not completely understood but are thought to be composed of both hereditary (e.g., BRCA1 and BRCA2 genes) and environmental factors (e.g., high fat diets, obesity, and smoking).

Lung cancer is one of the most common cancer worldwide, the third most commonly diagnosed cancer in the United States, and by far the most frequent cause of cancer deaths (Spiro et al., 2002, Am. J. Respir. Crit. Care Med. 166:1166-96; Jemal et al., 2003, CA Cancer J. Clin. 53:5-26). Cigarette smoking is believed responsible for an estimated 87% of all lung cancers making it the most deadly preventable disease. Lung cancer is divided into two major types that account for over 90% of all lung cancers: small cell lung cancer (SCLC) and non-small cell lung cancer (NSCLC). SCLC accounts for 15-20% of cases and is characterized by its origin in large central airways and histological composition of sheets of small cells with little cytoplasm. SCLC is more aggressive than NSCLC, growing rapidly and metastasizing early and often. NSCLC accounts for 80-85% of all cases and is further divided into three major subtypes based on histology: adenocarcinoma, squamous cell carcinoma (epidermoid carcinoma), and large cell undifferentiated carcinoma.

Lung cancer typically presents late in its course, and thus has poor prognosis. Although surgery offers the best chance of a cure, only a small fraction of lung cancer patients are eligible with the majority relying on chemotherapy and radiotherapy. Despite attempts to manipulate the timing and dose intensity of these therapies, survival rates have increased little over the last 15 years (Spiro et al., 2002, Am. J. Respir. Crit. Care Med. 166:1166-96). siRNA therapeutics could be a potential approach for lung cancer therapy.

Colorectal cancer is the third most common cancer and the fourth most frequent cause of cancer deaths worldwide (Weitz et al., 2005, Lancet 365:153-65). Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Colorectal carcinoma has a tendency to invade locally by circumferential growth and elsewhere by lymphatic, hematogenous, transperitoneal, and perineural spread. The most common site of extra-lymphatic involvement is the liver, with the lungs the most frequently affected extra-abdominal organ. Other sites of hematogenous spread include the bones, kidneys, adrenal glands, and brain. The current staging system for colorectal cancer is based on the degree of tumor penetration through the bowel wall and the presence or absence of nodal involvement. This staging system is defined by three major Duke's classifications: Duke's A disease is confined to submucosa layers of colon or rectum; Duke's B disease has tumors that invade through muscularis propria and can penetrate the wall of the colon or rectum; and Duke's C disease includes any degree of bowel wall invasion with regional lymph node metastasis. While surgical resection is highly effective for early-stage colorectal cancers, providing cure rates of 95% in Duke's A patients, the rate is reduced to 75% in Duke's B patients and the presence of positive lymph node in Duke's C disease predicts a 60% likelihood of recurrence within five years. Treatment of Duke's C patients with a post-surgical course of chemotherapy reduces the recurrence rate to 40%-50%, and is now the standard of care for these patients.

Epithelial carcinomas of the head and neck arise from the mucosal surfaces in the head and neck area and are typically squamous cell in origin. This category includes tumors of the paranasal sinuses, the oral cavity, and the nasopharynx, oropharynx, hypopharynx, and larynx. Traditional modes of therapy (radiation therapy, chemotherapy, and hormonal therapy), while useful, have been limited by the emergence of treatment-resistant cancer cells. Clearly, new approaches are needed to identify targets for treating head and neck cancer and cancer generally.

Breast cancer is the most common cancer in woman, with an estimate 12% of women at risk of developing the disease during their lifetime. Although mortality rates have decreased due to earlier detection and improved treatments, breast cancer remains a leading cause of death in middle-aged women. Furthermore, metastatic breast cancer is still an incurable disease. On presentation, most patients with metastatic breast cancer have only one or two organ systems affected, but as the disease progresses, multiple sites usually become involved. The most common sites of metastatic involvement are locoregional recurrences in the skin and soft tissues of the chest wall, as well as in axilla and supraclavicular areas. The most common site for distant metastasis is the bone (30-40% of distant metastasis), followed by the lungs and liver. And although only approximately 1-5% of women with newly diagnosed breast cancer have distant metastasis at the time of diagnosis, approximately 50% of patients with local disease eventually relapse with metastasis within five years. At present the median survival from the manifestation of distant metastases is about three years.

Current methods of diagnosing and staging breast cancer include the tumor-node-metastasis (TNM) system that relies on tumor size, tumor presence in lymph nodes, and the presence of distant metastases as described in the American Joint Committee on Cancer: AJCC Cancer Staging Manual. Philadelphia, Pa.: Lippincott-Raven Publishers, 5th ed., 1997, pp 171-180, and in Harris, J R: “Staging of breast carcinoma” in Harris, J. R., Hellman, S., Henderson, I. C., Kinne D. W. (eds.): Breast Diseases. Philadelphia, Lippincott, 1991. These parameters are used to provide a prognosis and select an appropriate therapy. The morphologic appearance of the tumor may also be assessed but because tumors with similar histopathologic appearance can exhibit significant clinical variability, this approach has serious limitations. Finally, assays for cell surface markers can be used to divide certain tumor types into subclasses. For example, one factor considered in the prognosis and treatment of breast cancer is the presence of the estrogen receptor (ER) as ER-positive breast cancers typically respond more readily to hormonal therapies such as tamoxifen or aromatase inhibitors than ER-negative tumors. Yet these analyses, though useful, are only partially predictive of the clinical behavior of breast tumors, and there is much phenotypic diversity present in breast cancers that current diagnostic tools fail to detect and current therapies fail to treat.

Despite multiple causative factors of breast cancer, the survival and growth of the breast cancer cells largely depend upon the common set of molecular signaling pathways in the cells.

Prostate cancer is the most common cancer in men in the developed world, representing an estimated 33% of all new cancer cases in the U.S., and is the second most frequent cause of death (Jemal et al., 2003, CA Cancer J. Clin. 53:5-26). Since the introduction of the prostate specific antigen (PSA) blood test, early detection of prostate cancer has dramatically improved survival rates, and the five-year survival rate for patients with local and regional stage prostate cancers at the time of diagnosis is nearing 100%. Yet more than 50% of patients will eventually develop locally advanced or metastatic disease (Muthuramalingam et al., 2004, Clin. Oncol. 16:505-16). Currently radical prostatectomy and radiation therapy provide curative treatment for the majority of localized prostate tumors. However, therapeutic options are very limited for advanced cases. For metastatic disease, androgen ablation with luteinising hormone-releasing hormone (LHRH) agonist alone or in combination with anti-androgens is the standard treatment. Yet despite maximal androgen blockage, the disease nearly always progresses with the majority developing androgen-independent disease. At present there is no uniformly accepted treatment for hormone refractory prostate cancer, and chemotherapeutic regimes are commonly used (Muthuramalingam et al., 2004, Clin. Oncol. 16:505-16; Trojan et al., 2005, Anticancer Res. 25:551-61).

Cancer arises from dysregulation of the mechanisms that control normal tissue development and maintenance, and increasingly stem cells are thought to play a central role (Beachy et al., 2004, Nature 432:324). During normal animal development, cells of most or all tissues are derived from normal precursors, called stem cells (Morrison et al., 1997, Cell 88:287-98; Morrison et al., 1997, Curr. Opin. Immunol. 9:216-21; Morrison et al., 1995, Annu. Rev. Cell. Dev. Biol. 11:35-71). Stem cells are cells that: (1) have extensive proliferative capacity; 2) are capable of asymmetric cell division to generate one or more kinds of progeny with reduced proliferative and/or developmental potential; and (3) are capable of symmetric cell divisions for self-renewal or self-maintenance. The best-known example of adult cell renewal by the differentiation of stem cells is the hematopoietic system where developmentally immature precursors (hematopoietic stem and progenitor cells) respond to molecular signals to form the varied blood and lymphoid cell types. Other cells, including cells of the gut, breast ductal system, and skin are constantly replenished from a small population of stem cells in each tissue, and recent studies suggest that most other adult tissues also harbor stem cells, including the brain.

Solid tumors are composed of heterogeneous cell populations. For example, breast cancers are a mixture of cancer cells and normal cells, including mesenchymal (stromal) cells, inflammatory cells, and endothelial cells. Classic models of cancer hold that phenotypically distinct cancer cell populations all have the capacity to proliferate and give rise to a new tumor. In the classical model, tumor cell heterogeneity results from environmental factors as well as ongoing mutations within cancer cells resulting in a diverse population of tumorigenic cells. This model rests on the idea that all populations of tumor cells would have some degree of tumorigenic potential. (Pandis et al., 1998, Genes, Chromosomes & Cancer 12:122-129; Kuukasjrvi et al., 1997, Cancer Res. 57:1597-1604; Bonsing et al., 1993, Cancer 71:382-391; Bonsing et al., 2000, Genes Chromosomes & Cancer 82: 173-183; Beerman H et al., 1991, Cytometry. 12:147-54; Aubele M & Werner M, 1999, Analyt. Cell. Path. 19:53; Shen L et al., 2000, Cancer Res. 60:3884).

An alternative model for the observed solid tumor cell heterogeneity is that solid tumors result from a “solid tumor stem cell” (or “cancer stem cell” from a solid tumor) that subsequently undergoes chaotic development through both symmetric and asymmetric rounds of cell division. In this stem cell model, solid tumors contain a distinct and limited (possibly even rare) subset of cells that share properties with normal “stem cells” in that they extensively proliferate and efficiently give rise both to additional solid tumor stem cells (self-renewal) and to the majority of within a solid tumor that lack tumorigenic potential. Indeed, mutations within a long-lived stem cell population can initiate the formation of cancer stem cells that underlie the growth and maintenance of tumors and whose presence contributes to the failure of current therapeutic approaches.

The stem cell nature of cancer was first revealed in the blood cancer, acute myeloid leukemia (AML) (Lapidot et al., 1994, Nature 17:645-8). More recently it has been demonstrated that malignant human breast tumors similarly harbor a small, distinct population of cancer stem cells enriched for the ability to form tumors in immunodeficient mice. An ESA+, CD44+, CD24−/low, Lin-cell population was found to be 50-fold enriched for tumorigenic cells compared to unfractionated tumor cells (Al-Hajj et al., 2003, PNAS 100:3983-8). Furthermore, a similar population is also present in colon cancers.

While various cancer types have unique biological mechanisms and cellular processes and etiological characteristics, it is through amassing large volume of information on the processes over the past century, and through mining such information, that researchers are able to now seek mechanisms of therapeutic intervention that target relatively early processes in cancer development to prevent progression of the disease more efficiently.

A cellular transformation from a seemingly normal cell can occur from a single genetic mutation in a cell, caused by an external chemical or environmental effect, or by spontaneous mutation, or by genetically inherited mutation. Irrespective of whether it is a result of a single, multiple, simultaneous, spontaneous, induced or inherited cause, the transformation is accompanied by concurrent loss or dysregulation of mechanisms that tightly control the cell division process.

By addressing one of the root causes, a cancer may be cured, for example overcoming the underlying genetic abnormalities that make cells cancerous and give rise to this devastating disease in the first place. However, certain nodal mechanisms that govern normal functioning and propagation of cells could also be targeted for effective control of a wider variety of cancer types.

The present disclosure offers therapeutic compositions and methods that are directed towards intervening a nodal mechanism that is found to be common in a large variety of cancers. The approach may involve modulating the expression of protein or proteins that act as master regulators to suppress tumorigenesis in normal cells, but become drivers of cancer cell formation when expressed abnormally. In one aspect, the present disclosure involves compositions and methods that effectively target a large variety of cancer for therapy, by targeting aberrant expression of one or more PRDM family members.

### Disregulation of PRDM Family Members in Various Cancers

PRDF1 and RIZ1 homology domain containing (PRDMs) are a subfamily of Kruppel-like zinc finger proteins controlling key processes in various cancer types. There is evidence of widespread upregulation of some PRDM family members and isoforms or variants across multiple tumor types. This along with their somatic dispensability make some PRDM family members, and in particular certain isoforms or variant forms within the family excellent therapeutic candidates with broad therapeutic window and limited toxic side effects.

PRDMs exhibit unique structural and functional dualities: (a) most of these proteins comprise a PR domain and ZNF arrays, a structural feature that combines a SET-like domain known as a PR domain, typically found in methyltransferases, with a variable array of C2H2 zinc fingers (ZNF) characteristic of DNA-binding transcription factors; (b) some of these proteins may be transcriptional activators/repressors, while their physiological function is context- and cell-dependent; mechanistically, some PRDMs have a PKMT activity and directly catalyze histone lysine methylation, while others are rather pseudomethyltransferases and act by recruiting transcriptional cofactors; (c) several of the PRDM proteins may be oncogenes or tumor suppressors—their pathological function depends on the specific PRDM isoform expressed during tumorigenesis.

All PRDM proteins share a characteristic structure that brings together aSET [Su(var)3-9, Enhancer-of-zeste and Trithorax]-like domain called a PR [PRDF1 (positive regulatory domain I-binding factor 1) domain, a RIZ1 (retinoblastoma protein-interacting zinc finger gene 1)] domain, and a variable array of Cys2-His2 (C2H2) zinc fingers (ZNF). Notable exceptions to this general structure include rare splice variants of PRDM7 and PRDM11 that are C2H2 zinc finger-deficient [3] and several PR-less isoforms, containing only the C-terminal zinc finger domains. A Q-rich, unstructured domain is uniquely present at the C terminus of PRDM10. Consistent with the conserved role of this domain in transcriptional activation, its deletion leads to downregulation of PRDM10-regulated genes.

PR/SET ([Su(var)3-9, enhancer-of-zeste and trithorax] domain containing family of methyltransferases (PRDM) are emerging through current investigations to play a major role in a large variety of cancers, having some members that are protective against cancer, and some members that are upregulated in cancer and promote transformation. Critical regulation of certain cancer promoting PRDM family members and isotypes might be an important therapeutic strategy.

In general, PRDM2 belongs to the positive regulatory domain (PRDM) gene family, a subfamily of Kruppel-like zinc finger gene products currently including 19 members in humans. PR domains have a protein-binding interface, and some of them can accommodate the universal methyl donor S-adenosyl methionine (SAM), therefore functioning as lysine methyltransferases (KMTs) PRDM (PRDI-BF1 and RIZ homology domain containing) protein family members belong to a superfamily of histone/protein methyltransferases (PRDMs), which are characterized by the conserved N-terminal PR domain, with methyltransferase activity and zinc finger arrays at the C-terminus. Members of this class are characterized by the presence of a PR domain and a variable number of Zn-finger repeats. Experimental evidence has shown that the PRDM proteins play an important role in gene expression regulation, modifying the chromatin structure either directly, through the intrinsic methyltransferase activity, or indirectly through the recruitment of chromatin remodeling complexes. PRDM proteins function as transcription factors, governing the expression of a vast array of other genes involved in developmental processes including growth, differentiation, proliferation, mobility, and survival. PRDM proteins function by tethering transcription factors to target gene promoters or by recognition of specific DNA consensus sequences via the Zinc-finger domains (Rienzo et al., Front. Oncol., 29 Jan. 2021 Sec Molecular and Cellular Oncology https://doi.org/10.3389/fonc.2020.583533). Of note, PRDM proteins contribute to many developmental processes, driving cell proliferation, differentiation, and maturation events by specifying cell fate choice or maintaining cell specialization through transduction of several cell signals. PRDM proteins are therefore master transcriptional regulators, driving and maintaining cell state transitions in response to developmental signals. Some PRDM proteins normally function as tumor suppressors, if their expression becomes dysregulated, or certain isoforms become expressed in abnormal proportions, these changes may become drivers of cancer onset and progression. Many cancer types are strongly associated with dysfunction of particular PRDM proteins. In such tumors, the normal PRDM protein may be expressed at levels that are abnormally low. PRDM proteins have a dual action: they mediate the effect induced by different cell signals like steroid hormones and control the expression of growth factors. PRDM proteins therefore have a pivotal role in the transduction of signals that control cell proliferation and differentiation and consequently neoplastic transformation. (Zazzo et al., Biology (Basel). 2013 March; 2(1): 107-141). The PR domain shares high homology with the catalytic SET (Suppressor of variegation 3-9, Enhancer of zeste and Trithorax) domain that defines a group of histone methyltransferases (Xiao B. et al., Curr. Opin. Struct. Biol. 2003; 13:699-705). In the human genome there are 17 genes encoding for proteins with a PR/SET and all of them but PRDM11 have a variable number of Zn-finger domains (Fumasoni I. et al., BMC Evol. Biol. 2007; 7:187). PRDM proteins have a pivotal role in the transduction of signals that control cell proliferation and differentiation and consequently neoplastic transformation (Fog C. K et al., BioEssays. 2011; 34:50-60). A common characteristic of PRDM family genes is the expression of different molecular forms by alternative splicing or by the action of different promoters. Furthermore, some genes of this family are expressed as two alternative forms, one lacking the PR domain (PR-minus) but otherwise identical to the other PR-containing product (PR-plus) (PRDM1, PRDM2, PRDM3, PRDM16) [(Gyory et al., J. Immunol, 2003, 170:3125-3133), (Liu L., et al., J. Biol. Chem. 1997, 272:2984-2991)]. Other genes encode for proteins that differ for the presence or absence of Zn-finger domains (PRDM6, PRDM9).

PRDM1 and PRDM2, initially identified as Blimp-1 (B lymphocyte-induced maturation protein-1) and RIZ (Retinoblastoma interacting zinc finger protein) respectively, have two promoters that encode for a PR-plus and a PR-minus isoform. PRDM1 promoters are localized upstream of exon 1 and exon 4 respectively. These transcriptional start sites at two promoters guide: PRDI-BF1 (Positive regulatory domain I-binding factor 1) a (PR-plus) e PRDI-BF1β (PR-minus) that differ only by the PR domain presence. Similarly, to PRDM1, PRDM2 expresses two proteins, PRDM2a/RIZ1 (PR-plus) and PRDM2b/RIZ2 (PR-minus), by differential transcription initiated by the two promoters. One promoter of PRDM2 is located upstream of the open reading frame in a region including exon 1a and a second promoter is located within intron 5 and exon 6. PRDM2 was first identified in independent studies as retinoblastoma interacting zinc-finger protein (RIZ) and as GATA-3 binding protein through functional screenings of human cDNA libraries (Sorrentino, A., et al., 2018, Volume 1861, Issue 7, Pages 657-671). Later, a PRDM2 variant, named MTE-binding protein zinc-finger type (MTB-ZF), was isolated from a human monocytic leukemia cell line cDNA expression library.

TABLE 1
PRDM genes and proteins

| | | Methyl transferase |
|---|---|---|
| | Cellular | activity |
| Gene/Protein | localization | (if/where known) |
| PRDM1(BLIMP1) / PR domain zinc finger | nucleocytoplasm | — |
| protein 1 | | |
| PRDM 2(RIZ) / PR domain zinc finger | Isoform: RIZ1: | H3K9 HMT activity |
| protein 2 (MTE binding protein) | nucleus | |
| | Isoform: RIZ2: | — |
| | nucleus | |
| PRDM3 / MIDS1 and EVI1 complex locus | Isoform 1: nucleus | H3K9me1 |
| protein 1 | Isoform 2: nucleus | |
| | Isoform 3: nucleus | |
| | Isoform 4: nucleus | |
| | Isoform 5: nucleus | |
| | Isoform 6: nucleus | |
| PRDM4 / PR domain zinc finger protein 4 | nucleus | — |
| PRDM5/ PR domain zinc finger protein 5 | Isoform 1: nucleus | — |
| | Isoform 2: nucleus | |
| | Isoform 3: nucleus | |
| PRDM6 / Putitive histone, lysine N-methyl | Isoform 1: nucleus | H4K20 |
| transferase PRDM6 | Isoform 2: nucleus | |
| | Isoform 3: nucleus | |
| PRDM7/ Probable histone, lysine N-methyl | Isoform 1: nucleus | — |
| transferase PRDM7 | Isoform 2: nucleus | |
| | Isoform 3: nucleus | |
| PRDM8/ PR domain zinc finger protein 8 | Isoform 1: nucleus | H3K9 |
| | Isoform 2: nucleus | |
| PRDM9/ histone N-methyl transferase PRDM9 | nucleus | H3K4-me3 |
| PRDM10/PR domain zinc finger protein 10 | Isoform 1: nucleus | — |
| | Isoform 2: nucleus | |
| | Isoform 3: nucleus | |
| | Isoform 4: nucleus | |
| | Isoform 5: nucleus | |
| | Isoform 6: nucleus | |
| PRDM11/PR domain zinc finger protein 11 | nucleus | — |
| PRDM12/PR domain zinc finger protein 12 | nucleus | — |
| PRDM13/PR domain zinc finger protein 13 | nucleus | — |
| PRDM14/PR domain zinc finger protein 14 | nucleus | — |
| PRDM15/PR domain zinc finger protein 15 | nucleus | — |
| PRDM16/PR domain zinc finger protein 16 | Isoform 1: nucleus | H3K9me1 |
| | Isoform 2: nucleus | |
| | Isoform 3: nucleus | |
| PRDM17// PR domain zinc finger protein 17 | nucleus | — |

Thus far, enzymatic activity has been experimentally demonstrated only for a few family members: PRDM9 (directed toward H3K4me3, H3K9me1/3, H3K18me1, H3K36me3 and H4K20me1/2), PRDM2, PRDM3 and PRDM16 (H3K9me1) and PRDM8 (H3K9me3) (Xie, M. et al., J. Biol. Chem., 1997, 272:42, 2636-26366). Additionally, activity has been reported for PRDM6, even though the nature of this activity requires further elucidation.

PRDM1 was shown to function as a tumor suppressor in B-cell lymphomas. The PRDM1 gene has two alternative promoters, transcribing two isoforms (i.e., PRDM1a and PRDM1b) with opposite functions, but both still able to bind to DNA (a phenomenon described as ‘Yin and Yang’). The PRDM1 comprises a C/T-rich consensus characterized by two consecutive [ACTTTC] repeats (SEQ ID NO: 23) is consistent with the predicted consensus of ZNF1-4.

PRDM2, or retinoblastoma-binding protein (RIZ) RIZ encodes Rb-binding proteins and the PRDI-BF1/BLIMP1 transcription repressor, which promotes B-lymphocyte maturation. RIZ can have two isoforms, PRDM2a (RIZ1) and PRDM2b (RIZ2). Both the proteins are widely expressed in mammalian cells. RIZ1 is identical to RIZ2, except that it has an extra 201 residues at the amino terminus, which, in RIZ1 comprises PR domain. These extra residues in RIZ1 is known to confer specific function to RIZ1 protein. An internal promoter generates RIZ2, that lacks the RIZ1 PR domain. The PR domain represents the major functional motif within the RIZ1 amino-terminal region. PR domain is a derivative of SET domain and may function as the protein binding interface in the regulation of chromatin-mediated gene expression. RIZ1 and RIZ2 isoforms can function may have opposing functions in cancer and are expressed indifferent contexts. PRDM2 RIZ 1 isoform may have tumor suppressor function, whereas, PRDM2-RIZ2 isoform may lack tumor suppressor function. In fact, it is highly upregulated in many cancer cell types, thereby disrupting the normal ration of Riz1-to-RIZ2 expression in cells. The proportionately higher expression of RIZ2 over RIZ1 in cancer cells when compared to normal (tissue matched non-cancer) cells may be highly correlated with the transformative process.

PRDM3 also has two isoforms. The short isoform of PRDM3, known as EVI1, is a potent oncogene in several leukemia subtypes. PRDM3/EVI1 has two sets of ZNFs, one at the N- and one at the C terminus of the protein, comprised of seven and three ZNF tandem repeats, respectively. Previous studies have demonstrated that the PRDM3/EVI1 N-terminal ZNF domain recognizes a GATA-like motif [GAC/TA]X0-6[GAT/CA], while the C terminus binds to an ETS-like motif [GAA/TGAT/G], respectively. PRDM4 is a nonessential transcriptional regulator during development. PRDM5 may function a tumor suppressor gene in a multitude of cancer types, whose expression is epigenetically silenced through CpG methylation. PRDM5's tumor sup-pressor function involves its ability to negatively regulate WNT signaling through transcriptional regulation of WNT reporters and WNT-responsive genes (Deng Q & Huang S (2004), Oncogene 23, 4903-4910. Likewise, PRDM 16 expresses two isoforms, one longer (PRDM16) comprising the PR domain and one shorter (MEL1), which lacks the PR domain and it is the shorter isoform that acts as a dominant-negative isoform in cancer. PRDM9 is known to play a role in oncogenesis.

### Inhibiting the Inhibitor

PRDM1 was shown to function as a tumor suppressor in B-cell lymphomas. The PRDM1 gene has two alternative promoters, transcribing two isoforms (i.e., PRDM1a and PRDM1b) with opposite functions, but both still able to bind to DNA (a phenomenon described as ‘Yin and Yang’). The PRDM1 comprises a C/T-rich consensus characterized by two consecutive [ACTTTC] repeats (SEQ ID NO: 23) is consistent with the predicted consensus of ZNF1-4.

PRDM2, or retinoblastoma-binding protein (RIZ) RIZ encodes Rb-binding proteins and the PRDI-BF1/BLIMP1 transcription repressor, which promotes B-lymphocyte maturation. RIZ can have two isoforms, PRDM2a (RIZ1) and PRDM2b (RIZ2). Both the proteins are widely expressed in mammalian cells. RIZ1 is identical to RIZ2, except that it has an extra 201 residues at the amino terminus, which, in RIZ1 comprises PR domain. These extra residues in RIZ1 is known to confer specific function to RIZ1 protein. An internal promoter generates RIZ2, that lacks the RIZ1 PR domain. The PR domain represents the major functional motif within the RIZ1 amino-terminal region. PR domain is a derivative of SET domain and may function as the protein binding interface in the regulation of chromatin-mediated gene expression. RIZ1 and RIZ2 isoforms can function may have opposing functions in cancer and are expressed indifferent contexts. PRDM2 RIZ 1 isoform may have tumor suppressor function, whereas, PRDM2-RIZ2 isoform may lack tumor suppressor function. In fact, it is highly upregulated in many cancer cell types, thereby disrupting the normal ration of RIZ1-to-RIZ2 expression in cells. The proportionately higher expression of RIZ2 over RIZ1 in cancer cells when compared to normal (tissue matched non-cancer) cells may be highly correlated with the transformative process.

PRDM3 also has two isoforms. The short isoform of PRDM3, known as EVI1, is a potent oncogene in several leukemia subtypes. PRDM3/EVI1 has two sets of ZNFs, one at the N- and one at the C terminus of the protein, comprised of seven and three ZNF tandem repeats, respectively. Previous studies have demonstrated that the PRDM3/EVI1 N-terminal ZNF domain recognizes a GATA-like motif [GAC/TA]X0-6[GAT/CA], while the C terminus binds to an ETS-like motif [GAA/TGAT/G], respectively. PRDM4 is a nonessential transcriptional regulator during development. PRDM5 may function a tumor suppressor gene in a multitude of cancer types, whose expression is epigenetically silenced through CpG methylation. Likewise, PRDM 16 expresses two isoforms, one longer (PRDM16) comprising the PR domain and one shorter (MEL1), which lacks the PR domain and it is the shorter isoform that acts as a dominant-negative isoform in cancer. PRDM9 is known to play a role in oncogenesis.

In some aspects, the present disclosure discloses targeting one or more isoforms of PRDM that plays a predominant role associated with cancer.

One of the primary challenges in developing a therapeutic that targets the PRDMs is that the PRDMs are difficult drug targets. The ‘Yin and Yang’ regulation entails that the oncogenic isoforms (i.e., PRDM1b, EVI1, MEL1) that are deprived of the druggable PR domain. (Di Tullio F. et al., The FEBS Journal 289(2022) 1256-1275). This had led major scientific and therapeutic efforts to concentrate on the category of oncogenic PRDMs in cancer, which are expressed as full length, hence retaining the druggable PR domain. Among the PRDMs showing an oncogenic function, extensive molecular characterization has been provided only for PRDM9, for PRDM14, PRDM15.

In general, most PRDMs are either silenced or deleted in cancer through an array of genetic (i.e., polymorphisms, frameshift/inactivating mutations, and chromosomal deletion) and epigenetic (i.e., DNA methylation and transcriptional silencing) mechanisms. For instance, genomic regions containing PRDM1(6q21-q22.1), PRDM2(1p36), and PRDM4(12q23-q24.1) are frequently deleted in cancer. Frameshift and inactivating mutations identified in PRDM1, PRDM2, PRDM3, PRDM8, and PRDM11 have been linked to transformation. In addition, an increasing number of studies have reported downregulation of several PRDMs at the transcriptional level via a variety of epigenetic mechanisms involving DNA methylation, histone post-translational modifications, and micro-RNAs, leading to deregulation of the local chromatin structure at PRDM-associated regulatory regions. Therefore, the predominant focus in PRDM related therapeutic development has been concentrated on induction or upregulation of these PRDMs that are deficient in cancer, rather than addressing another phenomenon in which the subtle balance of the full length versus the truncated, shorter isoform of some PRDM genes is disturbed in cancer cells.

The present disclosure is based on an important finding that a balance between RIZ1 and RIZ2 levels in mammalian cells is a determinant of an unperturbed normal life cycle of the cell, and an imbalance of the same leads to cell cycle changes leading to a hyper-proliferative disorder, e.g., a hyperplasia or cancer. The present disclosure is based on the further breakthrough development that RIZ2 can be specifically targeted to address the imbalance, and thereby to prevent or ameliorate a hyper-proliferative disorder, such as a cancer.

Of particular interest are cancer treatments that focus on altering the expression of the PRDM class of tumor suppressor genes. The PRDMs are a family of genes and proteins that control cell growth, proliferation, survival and mobility. Studies show that changes in the expression and activity of the PRDMs are commonly among the first changes in normal cells that lead to cancer cell formation. Aberrant expression of PRDM genes is strongly implicated in a wide variety of cancer types. The PRDMs have been implicated as causative genes and proteins in solid tumors such as breast, colon, gastric, liver, lung, melanoma, prostate and other cancers, as well as in blood cancers such as leukemia, lymphoma and myeloma.

A method of treating a cancer is hereby contemplated, comprising inhibiting at least one PRDM molecule that is a shorter variant of two or more PRDM isoforms, wherein the other of the two or more isoforms, or variants comprise longer molecules comprising at least one active domain that is missing in the shorter variant. For example, the shorter variant of PRDM2 family is RIZ2. In some embodiments the inhibitor that inhibits a shorter PRDM variant alters the intracellular balance between the longer and shorter variant, wherein the imbalance between the longer and the shorter variant is associated with the disease of condition to be treated. In some embodiments the inhibitor inhibiting the shorter variant decreases the protein level of the shorter variant compared to the longer variant. In some embodiments, the inhibitor that inhibits the shorter PRDM variant can be a protein inhibitor, a peptide inhibitor, a small molecule inhibitor, an antibody or fragment thereof, an scFv, a bispecific or trispecific molecule, a diabody, a single chain antibody, an inhibitory nucleic acid, such as miRNA, or siRNA. In some embodiments, the disease or condition is cancer, and inhibiting the shorter variant may function as an anti-proliferative agent.

In one aspect of the invention, provided herein is a method for inhibiting a shorter isoform or variant inside a diseased cell, e.g., a cancer cell, comprising, administering an inhibitor of the shorter isoform in a pharmaceutical formulation that further comprises a targeting moiety that brings the inhibitor in contact with the diseased cell. In some embodiments, the targeting moiety is a cancer cell-targeting moiety. In some embodiments, the formulation comprises a nanoparticle, wherein the nanoparticle comprises the inhibitor and the targeting moiety, and wherein the nanoparticle displays the targeting moiety on the surface of the nanoparticle. The targeting moiety can bind to a target protein on a target cell (e.g., cancer cell) surface, and deliver the therapeutically active siRNA compound into the cancer cell. In some embodiments, the targeting moiety is a ligand, an antibody, an scFv, a single domain antibody, a single chain antibody, a diabody—that binds to a protein on a cancer cell surface.

An exemplary sequence of the N-terminal region comprising the PR domain in RIZ1, that is absent in RIZ2 is shown below, and a putative PR domain is marked in bold:

(SEQ ID NO: 19)
MNQNTTEPVAATETLAEVPEHVLRGLPEEVRLFPSAVDKTRIGVWATKPILKG
KKFGPFVGDKKKRSQVKNNVYMWEVYYPNLGWMCIDATDPEKGNWLRYVNWA
CSGEEQNLFPLEINRAIYYKTLKPIAPGEELLVWYNGEDNPEIAAAIEEERASARSKR
SSPKSRKGKKKSQENKNKGNKIQDIQLKTSEPDFTSAN.

RIZ2 is characterized by the absence of the segment designated as SEQ ID NO: 19 that is present in RIZ1.

An exemplary RIZ2 protein sequence is provided below.

(SEQ ID NO: 20)
MRDSAEGPKEDEEKPSASALEQPATLQEVASQEVPPELATPAPAWEPQ
PEPDERLEAAACEVNDLGEEEEEEEEEDEEEEEDDDDDELEDEGEEEAS
MPNENSVKEPEIRCDEKPEDLLEEPKTTSEETLEDCSEVTPAMQIPRTK
EEANGDVFETFMFPCQHCERKFTTKQGLERHMHIHISTVNHAFKCKYCG
KAFGTQINRRRHERRHEAGLKRKPSQTLQPSEDLADGKASGENVASKD
DSSPPSLGPDCLIMNSEKASQDTINSSVVEENGEVKELHPCKYCKKVFG
THTNMRRHQRRVHERHLIPKGVRRKGGLEEPQPPAEQAQATQNVYVP
STEPEEEGEADDVYIMDISSNISENLNYYIDGKIQTNNNTSNCDVIEME
SASADLYGINCLLTPVTVEITQNIKTTQVPVTEDLPKEPLGSTNSEAKK
RRTASPPALPKIKAETDSDPMVPSCSLSLPLSISTTEAVSFHKEKSVYL
SSKLKQLLQTQDKLTPAGISATEIAKLGPVCVSAPASMLPVTSSRFKRR
TSSPPSSPQHSPALRDFGKPSDGKAAWTDAGLTSKKSKLESHSDSPAWS
LSGRDERETVSPPCFDEYKMSKEWTASSAFSSVCNQQPLDLSSGVKQKA
EGTGKTPVQWESVLDLSVHKKHCSDSEGKEFKESHSVQPTCSAVKKRKP
TTCMLQKVLLNEYNGIDLPVENPADGTRSPSPCKSLEAQPDPDLGPGSG
FPAPTVESTPDVCPSSPALQTPSLSSGQLPPLLIPTDPSSPPPCPPVLT
VATPPPPLLPTVPLPAPSSSASPHPCPSPLSNATAQSPLPILSPTVSPS
PSPIPPVEPLMSAASPGPPTLSSSSSSSSSSSSFSSSSSSSSPSPPPLS
AISSVVSSGDNLEASLPMISFKQEELENEGLKPREEPQSAAEQDVVVQE
TFNKNFVCNVCESPFLSIKDLTKHLSIHAEEWPFKCEFCVQLFKDKTDL
SEHRFLLHGVGNIFVCSVCKKEFAFLCNLQQHQRDLHPDKVCTHHEFES
GTLRPQNFTDPSKAHVEHMQSLPEDPLETSKEEEELNDSSEELYTTIKI
MASGIKTKDPDVRLGLNQHYPSFKPPPFQYHHRNPMGIGVTATNFTTHN
IPQTFTTAIRCTKCGKGVDNMPELHKHILACASASDKKRYTPKKNPVPL
KQTVQPKNGVVVLDNSGKNAFRRMGQPKRLNFSVELSKMSSNKLKLNAL
KKKNQLVQKAILQKNKSAKQKADLKNACESSSHICPYCNREFTYIGSLN
KHAAFSCPKKPLSPPKKKVSHSSKKGGHSSPASSDKNSNSNHRRRTADA
EIKMQSMQTPLGKTRARSSGPTQVPLPSSSFRSKQNVKFAASVKSKKPS
SSSLRNSSPIRMAKITHVEGKKPKAVAKNHSAQLSSKTSRSLHVRVQKS
KAVLQSKSTLASKKRTDRFNIKSRERSGGPVTRSLQLAAAADLSENKRE
DGSAKQELKDFRNFL.

The sequence of SEQ ID NO: 20 aligns perfectly with RIZ1 at amino acid residue 202 for RIZ1 onwards. Shown below, the query sequence is RIZ1, and the subject sequence is RIZ2. For example, a PRDM2/RIZ1 is a sequence of that of NM_012231. For example, a PRDM2/RIZ2 is a sequence is that of NM_001007257. The sequence of NM_012231 is an exemplary human RIZ1 sequence. The sequence of NM_001007257 is an exemplary human RIZ2 sequence.

In one aspect, provided herein is a pharmaceutical composition for treating a cancer in a subject in need thereof, comprising: (i) a therapeutically effective amount of an inhibitory RNA molecule (siRNA) capable of binding to a PRDM2 transcript, and inhibiting translation of the PRDM2 RIZ2 transcript without inhibiting the translation of the full length RIZ1 transcript, such that effective ratio of RIZ1:RIZ2 proteins in the cancer cell is increased upon contacting the cell with the pharmaceutical composition comprising the siRNA.

In some embodiments, a pharmaceutical composition is hereby presented or contemplated herein which comprises an inhibitor suitable for the inhibition of the short isoform of PRDM3, known as EVI1, is a potent oncogene in several leukemia subtypes. In some embodiments, the inhibitor is an siRNA that selectively or preferentially inhibits the translation of the shorter transcript and over the full length PRDM3.

In some embodiments, a pharmaceutical composition is hereby presented or contemplated herein which comprises an inhibitor suitable for the inhibition of the short isoform of PRDM16 (MEL1 isoform), which lacks the PR domain and acts as a dominant-negative isoform in cancer.

In some embodiments, a PRDM9 transcript or translated product may be targeted from downregulation in a cancer by a pharmaceutical agent, such that the agent specifically downregulates or inhibits the PRDM9 shorter (inhibitory) isoform, and not the longer isoform.

Both RIZ1 and RIZ2 bind to GC-rich Sp-1-like DNA elements and repress transcription of the simian virus 40 early promoter, but RIZ1 is a more potent repressor than RIZ2, suggesting that the PR domain of RIZ1 modulates transcription (Huang, S., J. Biol. Chem., Vol. 273, No. 26, Issue of June 26, pp. 15933-15939).

RIZ1 proteins are expressed in normal tissues, but in many human malignant tissues and cancer cell lines, a reduction or absence of RIZ1 and/or an increase in RIZ2 expression levels have frequently been detected. As has been proposed for other PRDM proteins, this “yin-yang” imbalance in the amount of the two protein products may be important for neoplastic transformation. Moreover, an exclusive negative selection (specific genetically or epigenetically mediated downregulation) for RIZ1 versus RIZ2 seems to be a common feature for various human cancers. This observation suggests that RIZ1 may have tumor suppressor activity and that RIZ2 is necessary for oncogenesis by promoting cell proliferation through its mitogenic activity. RIZ2 putative intrinsic growth-promoting oncogenic properties have been linked to the first cluster of Zn-finger domains (residues 359-507) that are present in both RIZ1 and RIZ2. Indeed, stably transfected MCF7 cells expressing this cluster of Zn-fingers showed an increased proliferation rate compared to control cells, both in estrogen deprived conditions or upon estrogen stimulation, and showed higher expression levels of cyclin D1 and A and reduced responsiveness to the growth inhibitory effect of anti-estrogens. In addition, through a proteomic mass spectrometry-based approach, the forced expression of PRDM2 gene Zn-finger domains in MCF-7 cells was correlated to the differential expression of proteins associated with different types of carcinomas or involved in cell proliferation and differentiation, such as keratin K8, glycolytic enzyme α-enolase, acid protease cathepsin D and nucleoside diphosphate kinase A. Altered expression of these proteins may contribute to the observed higher proliferation rate. The oncogenic proprieties of RIZ2 are likely inhibited by the presence of the PR domain in RIZ1 protein. The drastic functional difference in chromatin regulation between RIZ1 and RIZ2, due to the PR domain conferring specific functions to RIZ1, may underlie their opposite roles in tumorigenesis.

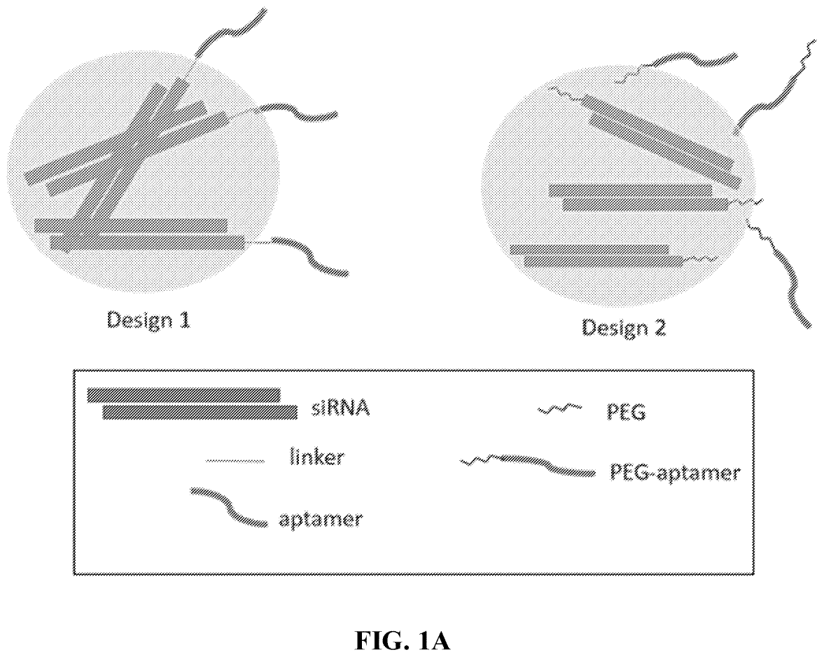

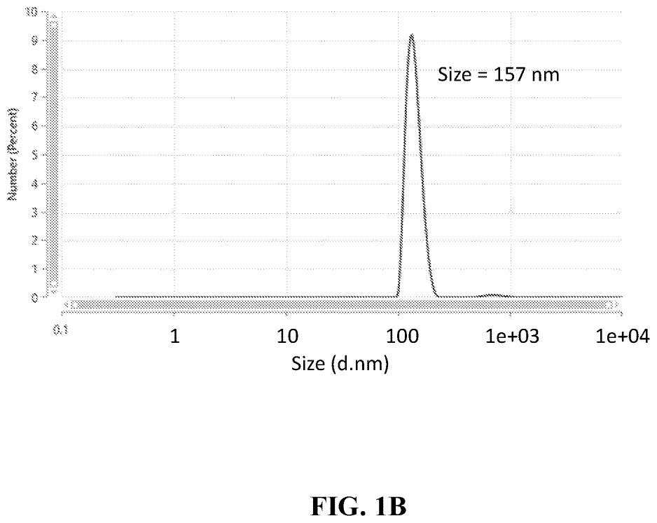

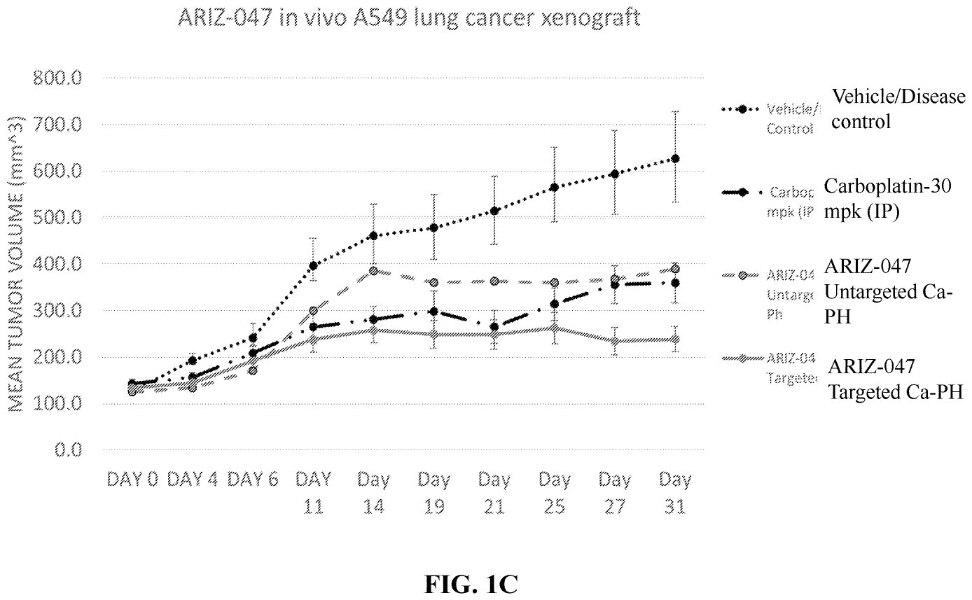

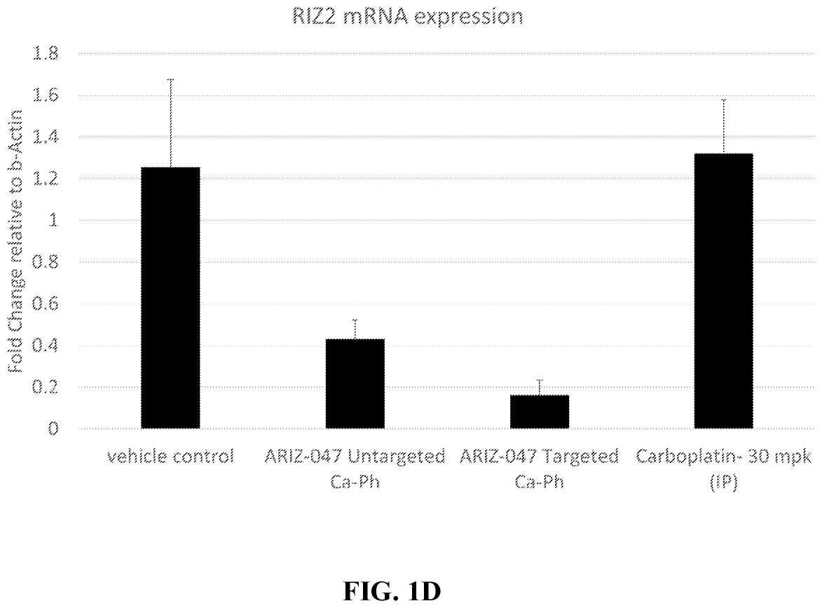

The imbalance in the respective amounts of RIZ1 and RIZ2 may be an important cause of malignancy, with the PR-positive isoform commonly lost or downregulated and the PR-negative isoform always being present at higher levels in cancer cells. Interestingly, the RIZ1 isoform also represents an important target of estradiol action downstream of the interaction with hormone receptor. Furthermore, the imbalance between the two products could also be a molecular basis for other human diseases. In one embodiment according to the invention as illustrated in FIG. 1, a drug delivery vehicle is equipped with a protective layer (for example, PEGylation of liposomes) to extend the drug lifetime in the bloodstream and shield the drug delivery system from destruction by the immune system. In some embodiments, the drug delivery system is specifically targeted to cancer cells via a ligand that targets a receptor or other moiety on the surface of a tumor cell or cancer stem cell. The ligand may be a protein, peptide or other class of molecule with the ability to bind to the targeted cancer cell with high specificity and affinity. This targeted drug delivery system should protect against damage to healthy cells, as well as concentrate the payload at the target site. Together this should increase efficacy (at lower drug doses) and improve the safety profile, resulting in a higher therapeutic index.

In other embodiments, the drug delivery vehicle may carry payloads in addition to the inhibitory siRNA, to potentiate the anti-cancer efficacy of the drug formulation. Such additional payloads may include, but are not limited to, large or small molecule chemotherapy agents.

PRDM2/RIZ1 may be regulated via methylation. In one aspect, RIZ2 hypermethylation may be targeted for cancer therapy. For instance, in a study, PRDM2/RIZ1 methylation frequency has been observed to be about 73% in tumor vs. 20% in distant lung tissue (Tan S. et al., Oncotarget and Therapy 2018: 11, 2991-3002).

The two RIZ isoforms regulate cellular functions in a “Yin-Yang” fashion (Di Zazzo, Biology 2016, 5, 54; doi:10.3390) whereby the two forms produce dual complimentary opposite reactions. Specifically, the RIZ1 protein plays the role of tumor suppressor, arresting cancer cells in the G2/M phase of the cell cycle and promoting apoptosis, while the RIZ2 protein acts as a proto-oncogene, promoting cell division. Thus, an imbalance in the amounts of RIZ1 and RIZ2 may be an important cause of cancer progression. That view is supported by research finding that silencing or deregulation of RIZ1 expression, associated with increased RIZ2 expression, has been observed in a variety of human cancers, including hepatoma, leukemia, malignant lymphoma, breast cancer, colorectal cancer, thyroid carcinoma and others.

In one aspect, the instant disclosure provides a method for regulating PRDM2 gene expression. In some embodiments, the method can regulate RIZ2 expression. In some embodiments, the method can be used to decrease RIZ2 expression. In some embodiments, the RIZ2 expression is decreased in a cell using the method described herein. In some embodiments, the method described herein is used to decrease RIZ2 expression in a cell within a mammalian system. In some embodiments, the mammalian system is a human system.

In some embodiments, provided herein is a method to decrease the level of RIZ2 in a cell, that shows aberrantly high expression of RIZ2 compared to a normal cell, wherein a normal cell is a healthy cell, or a cell collected from a healthy individual, wherein a healthy individual may be an individual who does not present clinical signs of a disease or a condition. In some embodiments, a cell that shows aberrantly high expression of RIZ2 is a cell that has a cell cycle regulation disorder. In some embodiments, a cell that shows aberrantly high expression of RIZ2 is a cell that exhibits hyperplasia. In some embodiments, a cell that shows aberrantly high expression of RIZ2 is a cancer cell.

In some embodiments, provided herein is a method of decreasing RIZ2 expression specifically in a cancer cell, or a cell exhibiting hyperplasia, wherein the cell exhibits an aberrantly high expression of RIZ2. In some embodiments, the cell that exhibits aberrantly high expression of RIZ2 also exhibits a decrease in expression of RIZ1 concurrently. In some embodiments, the method provided herein is capable of decreasing RIZ2 expression, and concurrently increase expression of RIZ1. In some embodiments, the method provided herein restores a RIZ1-RIZ2 balance in a cell. In some embodiments, the method provided herein allows upregulation of RIZ1 and downregulation of RIZ2. In some embodiments, the method provided herein allows upregulation of RIZ1 by downregulation of RIZ2. In some embodiments, the method provided herein comprises contacting the cell with a RIZ2 inhibitor.

Provided herein is a method of downregulation of RIZ2 in a cell expressing elevated RIZ2 levels than that of a normal cell, and simultaneously upregulating RIZ1 levels, wherein the cell expressing elevated RIZ2 levels than that of a normal cell also exhibits a decreased RIZ1 level than that of a normal cell, the method comprises contacting the cell with a RIZ2 inhibitor described in the disclosure. In some embodiments, a normal cell is a cell that exhibits a normal cell division cycle. In some embodiments, provided herein is a method of restoring RIZ1-RIZ2 balance in a cell. In some embodiments, the ratio of RIZ1 to RIZ2 mRNA expression levels are altered by greater than at least 1.1-fold. For example, in some embodiments, the ratio of RIZ1 to RIZ2 mRNA expression levels are altered by greater than at least 1.2-fold. In some embodiments, the ratio of RIZ1 to RIZ2 mRNA expression levels are altered by greater than at least 1.3-fold. In some embodiments, the ratio of RIZ1 to RIZ2 mRNA expression levels are altered by greater than at least 1.4-fold. In some embodiments, the ratio of RIZ1 to RIZ2 mRNA expression levels are altered by greater than at least 1.5-fold. In some embodiments, the ratio of RIZ1 to RIZ2 mRNA expression levels are altered by greater than at least 1.6-fold. In some embodiments, the ratio of RIZ1 to RIZ2 mRNA expression levels are altered by greater than at least 1.7-fold. In some embodiments, the ratio of RIZ1 to RIZ2 mRNA expression levels are altered by greater than at least 1.8-fold. In some embodiments, the ratio of RIZ1 to RIZ2 mRNA expression levels are altered by greater than at least 1.9-fold. In some embodiments, the ratio of RIZ1 to RIZ2 mRNA expression levels are alters the ratio of RIZ1 to RIZ2 mRNA expression levels by at least 2-fold.

In some embodiments, the method provided herein comprises contacting the cell with a composition comprising a Retinoblastoma Protein-Interacting Zinc Finger Protein 2 (RIZ2) inhibitor. In some embodiments, the RIZ2 inhibitor is a non-naturally occurring agent, e.g., a synthetic agent. In some embodiments, the RIZ2 inhibitor is a small molecule. In some embodiments, the small molecule is designed to be specific for inhibiting RIZ2 and not inhibiting RIZ1. In some embodiments, the RIZ2 inhibitor comprises a peptide. In some embodiments, the RIZ2 inhibitor is a peptide that specifically inhibits RIZ2 in a cell, and comprises at least about 4 amino acids, at least about 4 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, or at least about 20 amino acids, or more than 20 amino acids. In some embodiments, the RIZ2 inhibitor comprises a conjugated polypeptide. In some embodiments, the conjugated polypeptide comprises at least one bioactive polypeptide capable of decreasing RIZ2 level (RIZ2 inhibitor), and a moiety that confers stability, or passage across cell membrane or allows targeting the bioactive peptide to a specific cell, a tissue or an organ when applied in vivo. In some embodiments, the RIZ2 inhibitor comprises one or more polynucleotides. In some embodiments, RIZ2 inhibitor comprises a synthetic polynucleotide. In some embodiments, RIZ2 inhibitor comprises a synthetic polynucleotide such as an siRNA.

In some embodiments, the method comprises contacting the cell with a RIZ2 inhibitor wherein the RIZ2 inhibitor is present at an amount sufficient to decrease the RIZ2 level in the cell, and to increase the RIZ1 level in the cell such that the level of RIZ2 and RIZ1 are comparable to a normal cell, or such that the ratio of RIZ1 versus RIZ2 is brought back to levels similar to normal cell, i.e., a cell that has a normal proliferation rate, e.g., a normal cell cycle.

In some embodiments, the methods described herein is a method of inhibiting cell proliferation, comprising contacting a population of cells with a composition comprising a RIZ2 inhibitor.

In some embodiments, the methods described herein, when applied to a population of cells, reduces cell proliferation rate in the cell population from a hyperproliferative state to a proliferation rate similar to normal healthy cells.

In some embodiments, the methods described herein, when applied to a population of cells, results in a reduction of a cell number of the population of cells by at least 10%. In some embodiments, contacting the cells with a RIZ2 inhibitor results in a reduction of a cell number of the population of cells by at least about 15%, or by at least about 20%, at least about 25%, by at least about 30%, by at least about 35%, at least about 40%, at least about 45%, by at least about 50%, at least about 55%, at least about 60%, by at least about 65%, by at least about 70%, by at least about 75%, by at least about 80%, at least about 85%, or at least about 90%.

In some embodiments, contacting the cells with the RIZ2 inhibitor results in a reduction of a cell number of the population of cells by about 90%. In some embodiments, inhibiting cell proliferation comprises reducing the cell mass by at least 10%. In some embodiments, contacting the cells with a RIZ2 inhibitor results in a reduction of a cell mass of the population of cells by at least about 15%, or by at least about 20%, at least about 25%, by at least about 30%, by at least about 35%, at least about 40%, at least about 45%, by at least about 50%, at least about 55%, at least about 60%, by at least about 65%, by at least about 70%, by at least about 75%, by at least about 80%, at least about 85%, or at least about 90%.%. In some embodiments, contacting the cells with a RIZ2 inhibitor results in a reduction of a cell mass of the population of cells by at least about 15%, or by at least about 20%, at least about 25%, by at least about 30%, by at least about 35%, at least about 40%, at least about 45%, by at least about 50%, at least about 55%, at least about 60%, by at least about 65%, by at least about 70%, by at least about 75%, by at least about 80%, at least about 85%, or at least about 90%.

In some embodiments, inhibiting cell proliferation comprises reducing a cell mass by about 50%. In some embodiments, inhibiting cell proliferation comprises reducing a cell mass by about 60%. In some embodiments, inhibiting cell proliferation comprises reducing a cell mass by about 65%. In some embodiments, inhibiting cell proliferation comprises reducing a cell mass by about 70%. In some embodiments, inhibiting cell proliferation comprises reducing a cell mass by about 75%.

In some embodiments, the method described here comprises a method of treating a hyperproliferative disorder in a subject, e.g., a human subject, by administering to the subject a therapeutically effective amount of the RIZ2 inhibitor, wherein, when administered at a dose and suitable time interval reduced or ameliorated the hyperproliferative disorder in the subject. In some embodiments, the hyperproliferative disorder is cancer.

siRNA

Provided herein is an inhibitory RNA molecule suitable for inhibiting RIZ2 expression. In some embodiments, the specific RNA molecule is suitable for therapeutic applications. In some embodiments, the inhibitory RNA molecule is a synthetic small nucleic acid molecules, such as short interfering nucleic acid (siNA), short interfering RNA (siRNA), double-stranded RNA (dsRNA), micro-RNA (miRNA), and short hairpin RNA (shRNA) molecules capable of modulating gene expression in cells by RNA inference (RNAi). The siNA molecules of the invention can be unmodified or chemically modified. The use of chemically modified siNA can improve various properties of native siRNA molecules through increased resistance to nuclease degradation in vivo and/or improved cellular uptake. The chemically modified siNA molecules of the instant invention provide useful reagents and methods for a variety of therapeutic, cosmetic, cosmeceutical, prophylactic, diagnostic, agricultural, target validation, genomic discovery, genetic engineering and pharmacogenomic applications. In some embodiments, the inhibitory RNA molecule is an siRNA comprising a sense and antisense strand capable of hybridizing to form a duplex or double strand region.

In some embodiments, the siRNA comprises an antisense strand of at least 5, 6, 7, 8, 9, 10 contiguous nucleotides complementary and capable of binding (e.g., hybridizing) to at least 5, 6, 7, 8, 9, 10 contiguous nucleotides of the PRDM2 gene.

In some embodiments, the siRNA comprises an antisense strand of at least 5, 6, 7, 8, 9, 10 contiguous nucleotides complementary and capable of binding (e.g., hybridizing) to at least 5, 6, 7, 8, 9, 10 contiguous nucleotides of the RIZ1 or RIZ2 gene.

In some embodiments, the siRNA comprises an antisense strand of at least 5, 6, 7, 8, 9, 10 contiguous nucleotides complementary and capable of binding (e.g., hybridizing) to at least 5, 6, 7, 8, 9, 10 contiguous nucleotides of the RIZ2 gene.

In some embodiments, the siRNA comprises an antisense strand of at least 5, 6, 7, 8, 9, 10 contiguous nucleotides complementary and capable of binding (e.g., hybridizing) to at least 5, 6, 7, 8, 9, 10 contiguous nucleotides of the RIZ2 gene but not the RIZ1 gene.

In some embodiments, the siRNA binds (e.g., hybridizes) specifically to at least 5, 6, 7, 8, 9, 10 contiguous nucleotides of the RIZ2 gene, and predominantly affects RIZ2 gene expression without negatively affecting RIZ 1 expression levels. In some embodiments, the terms ‘negatively affecting an expression’ can indicate, inter alia, downregulating the expression of the said gene or gene product. For example, in some embodiments, a substance e.g., “a” negatively affecting an expression of another substance e.g., “b” could mean that the expression of the substance “b” is downregulated by 5%, 10%, 15%, 20%, 30%, 40% 50% or more in the presence of, as a consequence of, or in correlation with the presence of the substance “a”, wherein the expression could be mRNA expression, or the level of a translated protein product of the substance “b”. In this regard, in some embodiments, the siRNA that binds specifically to at least 5, 6, 7, 8, 9, 10 contiguous nucleotides of the RIZ2 gene and downregulates RIZ2 expression level, does not necessarily downregulate RIZ1 expression levels. For example, in some embodiments, after application of the siRNA that binds specifically to a RIZ2 transcript, the RIZ2 mRNA level in a cell can be downregulated by about 10%, about 20%, about 30%, about 40%, about 50%, about 60%, about 70%, about 80%, about 90% or about 91, 92, 93, 94, 95, 96, 97, 98, 99%; but the level of a RIZ1 mRNA is not appreciably downregulated in the same cell. In some embodiments, the after application of the siRNA that binds specifically to a RIZ2 transcript, the RIZ2 mRNA level in a cell can be downregulated by about 10%, about 20%, about 30%, about 40%, about 50%, about 60%, about 70%, about 80%, about 90% or about 91, 92, 93, 94, 95, 96, 97, 98, 99%; but the level of a RIZ1 mRNA may be upregulated by about 10%, about 20%, about 30%, about 40%, about 50% or more. In some embodiments, after application of the siRNA that binds specifically to a RIZ2 transcript, the RIZ2 mRNA level in a cell can be downregulated by about 10%, about 20%, about 30%, about 40%, about 50%, about 60%, about 70%, about 80%, about 90% or about 91, 92, 93, 94, 95, 96, 97, 98, 99%, and the ratio of RIZ1 mRNA to RIZ2 mRNA is increased in the cell. In some embodiments, after application of the siRNA that binds specifically to a RIZ2 transcript, the RIZ2 mRNA level in a cell can be downregulated by about 10%, about 20%, about 30%, about 40%, about 50%, about 60%, about 70%, about 80%, about 90% or about 91, 92, 93, 94, 95, 96, 97, 98, 99%, and the molar ratio of RIZ1 mRNA to RIZ2 mRNA is increased in the cell. In some embodiments, after application of the siRNA that binds specifically to a RIZ2 transcript, the RIZ2 mRNA level in a cell can be downregulated by about 10%, about 20%, about 30%, about 40%, about 50%, about 60%, about 70%, about 80%, about 90% or about 91, 92, 93, 94, 95, 96, 97, 98, 99%, and the molecular ratio of RIZ1 mRNA to RIZ2 mRNA is increased in the cell.

In some embodiments, the ratio of RIZ1 to RIZ2 transcripts is upregulated in a cell after application of the siRNA by about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 fold or more. In some embodiments, the ratio of RIZ1 to RIZ2 transcripts is increased in a cell after application of the siRNA by about 1.1-10 fold, 1.1-20 fold, 1.1-30 fold, or more.

In one aspect, the present disclosure provides one or more siRNA sequences capable of reducing (e.g., silencing) the expression of RIZ2 gene, without negatively affecting the expression of RIZ1 gene, and specifically, without reducing the expression of RIZ1 gene. More specifically, provided herein are siRNA sequences that reduce or inhibit the expression of a RIZ2 gene, and induces the expression of RIZ1 gene. In some embodiments, the siRNA sequences provided herein are capable of reducing, or inhibiting human RIZ2 expression, without inhibiting human RIZ1 expression. In some embodiments, the present disclosure is based on the surprising and unexpected finding that one or more siRNA targeting a common region of the RIZ2 and RIZ1 gene, predominantly inhibits RIZ2 but spares RIZ1, even though the siRNA shares homology to both the genes/gene products.

In some embodiments, the siRNA is designed to target a region between 1-500 nucleotides from the 5′-end of the RIZ2 mRNA. In some embodiments, the siRNA is designed to target a region between 10-500 nucleotides from the 5′-end of the RIZ2 mRNA. In some embodiments, the siRNA is designed to target a region between 100-500 nucleotides from the 5′-end of the RIZ2 mRNA. In some embodiments, the siRNA is designed to target a region between 200-500 nucleotides from the 5′-end of the RIZ2 mRNA. In some embodiments, the siRNA is designed to target a region between 500-1000 nucleotides from the 5′-end of the RIZ2 mRNA. In some embodiments the siRNA is designed to target a region between 200-2000 nucleotides from the 5′-end of the RIZ2 mRNA. In some embodiments, the siRNA is designed to target a region between 500-2500 nucleotides from the 5′-end of the RIZ2 mRNA. In some embodiments, the siRNA is designed to target a region between 1000-2500 nucleotides from the 5′-end of the RIZ2 mRNA. In some embodiments, the siRNA is designed to target a region between 1600-2200 nucleotides from the 5′-end of the RIZ2 mRNA. In some embodiments, the siRNA is designed to target a region between 1800-2200 nucleotides from the 5′-end of the RIZ2 mRNA. In some embodiments, the siRNA is designed to target a region between 1900-1950 nucleotides from the 5′-end of the RIZ2 mRNA. In some embodiments, the siRNA is designed to target a region between 1920-1940 nucleotides from the 5′-end of the RIZ2 mRNA. In some embodiments, the siRNA is designed to target a region between 2000-3500 nucleotides from the 5′-end of the RIZ2 mRNA. In some embodiments, the siRNA is designed to target a region between 1000-5000 nucleotides from the 5′-end of the RIZ2 mRNA. In some embodiments, the siRNA is designed to target a region between 1500-3000 nucleotides from the 5′-end of the RIZ2 mRNA. In some embodiments, the siRNA is designed to target a region between 2500-3500 nucleotides from the 5′-end of the RIZ2 mRNA. In some embodiments, the siRNA is designed to target a region between 3500-5000 nucleotides from the 5′-end of the RIZ2 mRNA. In some embodiments, the siRNA is designed to target a region between 4000-5000 nucleotides from the 5′-end of the RIZ2 mRNA. In some embodiments, the siRNA is designed to target a region between 4250-4500 nucleotides from the 5′-end of the RIZ2 mRNA. In some embodiments, the siRNA is designed to target a region between 4300-4400 nucleotides from the 5′-end of the RIZ2 mRNA. In some embodiments, the siRNA is designed to target a region between 4500-5000 nucleotides from the 5′-end of the RIZ2 mRNA. In some embodiments, the siRNA is designed to target a region between 4550-4700 nucleotides from the 5′-end of the RIZ2 mRNA. In some embodiments, the siRNA is designed to target a region between 4560-4600 nucleotides from the 5′-end of the RIZ2 mRNA. In some embodiments, the siRNA is designed to target a region between 4500-6000 nucleotides from the 5′-end of the RIZ2 mRNA. In some embodiments, the siRNA is designed to target a region between 5000-6140 nucleotides from the 5′-end of the RIZ2 mRNA. In some embodiments, the siRNA is designed to target a region between 6050-6155 nucleotides from the 5′-end of the RIZ2 mRNA. In some embodiments, the siRNA is designed to target a region between 6090-6120 nucleotides from the 5′-end of the RIZ2 mRNA.

Provided below is an exemplary list of sequences comprising sense and antisense strands of siRNA shown in the exemplary studies for the invention disclosed herein. For example, ARIZ-047 comprises a senses strand having a sequence of SEQ ID NO: 6 and an antisenses strand having a sequence of SEQ ID NO: 11.

TABLE 2
RIZ-specific siRNA

| | SEQ ID | Sequence | SEQ ID | Sequence |
|---|---|---|---|---|
| Name | NO: | (Sense strand) | NO: | (Antisense stranded) |
| ARIZ- | 1 | 5′- | 10 | 5′- |
| 011 | | GGGAGAGAUGAGAGAGAAAd | | UUUCUCUCUCAUCUCUCCCd |
| | | TdT-3′ | | TdT-3′ |
| ARIZ- | 6 | 5′Disulfide- | 11 | 5′P- |
| 047 | | GGGAGAGA4GAGAGAGAAAd | | 446C6C6CUCAUC6C6CCCdTdT-3′ |
| | | TdT-3′ (where 4 = | | (where 4 = 2′-methoxyuridine; |
| | | 2′-methoxyuridine) | | 6 = 2′- |
| | | | | Fluoro-deoxyuridine; bold + |
| | | | | underline = phosphorothioate) |
| ARIZ- | 2 | 5′- | 12 | 5′- |
| 012 | | GUGUAGUGCUGUAAAGAAAd | | UUUCUUUACAGCACUACACd |
| | | TdT-3′ | | TdT-3′ |
| ARIZ- | 3 | 5′- | 13 | 5′- |
| 013 | | GCAAAAUGUCGUCGAAUAAd | | UUAUUCGACGACAUUUUGCd |
| | | TdT-3′ | | TdT-3′ |
| ARIZ- | 4 | 5′- | 14 | 5′- |
| 014 | | UGGAAAAGGUGUCGACAAUd | | AUUGUCGACACCUUUUCCAd |
| | | TdT-3′ | | TdT-3′ |
| ARIZ- | 5 | 5′- | 15 | 5′- |
| 015 | | GGACUUCAGGAACUUCCUGd | | CAGGAAGUUCCUGAAGUCCd |
| | | TdT-3′ | | TdT-3′ |
| ARIZ- | 7 | 5′- | 16 | 5′P- |
| 062 | | CUUAAUAACUAGAGGAGAAd | | UUCUCCUCUAGUUAUUAAGd |
| | | TdT-3′ | | TdT-3′ |
| ARIZ- | 8 | 5′- | 17 | 5′P- |
| 063 | | CGCAAAGGAUACGAAAUCdT | | GAUUCCGUAUCCUUUGCCGd |
| | | dT-3′ | | TdT-3′ |
| ARIZ- | 9 | 5′- | 18 | 5′P- |
| 064 | | GGAUAAGCACUACGGCAAAd | | UUUGCCGUAGUGCUUAUCCd |
| | | TdT-3′ | | TdT-3′ |

Provided herein is a number of double stranded polynucleotide compositions comprising a sense strand and an antisense strand presented in Table 2, wherein the left column comprising the sense strand sequence for each double stranded polynucleotide pairs with an antisense strand sequence presented on the right column within the row. In some embodiments, the siRNA is 21 nucleotides in length. In some embodiments, the siRNA comprises a 1, 2, or 3 nucleotide single nucleotide overhang at the 5′- or the 3′-end of either the sense or antisense strand.

Therefore, in some embodiments, provided herein is a RIZ 2 inhibitor comprising a double stranded polynucleotide comprising a sense strand comprising a nucleotide sequence differing by no more than 4 nucleotides from SEQ TD NO: 1, and an antisense strand comprising a nucleotide sequence differing by no more than 4 nucleotides from SEQ TD NO: 10. In some embodiments, provided herein is a RIZ 2 inhibitor comprising a double stranded polynucleotide comprising a sense strand of SEQ TD NO: 1, and an antisense strand of SEQ TD NO: 10.

In some embodiments, provided herein is a RIZ 2 inhibitor comprising a double stranded polynucleotide comprising a sense strand comprising a nucleotide sequence differing by no more than 4 nucleotides from SEQ ID NO: 6, and an antisense strand comprising a nucleotide sequence differing by no more than 4 nucleotides from SEQ ID NO: 11. In some embodiments, provided herein is a RIZ 2 inhibitor comprising a double stranded polynucleotide comprising a sense strand of SEQ TD NO: 6, and an antisense strand of SEQ ID NO: 11.

In some embodiments, provided herein is a RIZ 2 inhibitor comprising a double stranded polynucleotide comprising a sense strand comprising a nucleotide sequence differing by no more than 4 nucleotides from SEQ ID NO: 2, and an antisense strand comprising a nucleotide sequence differing by no more than 4 nucleotides from SEQ ID NO: 12. In some embodiments, provided herein is a RIZ 2 inhibitor comprising a double stranded polynucleotide comprising a sense strand of SEQ ID NO: 2, and an antisense strand of SEQ ID NO: 12.

In some embodiments, provided herein is a RIZ 2 inhibitor comprising a double stranded polynucleotide comprising a sense strand comprising a nucleotide sequence differing by no more than 4 nucleotides from SEQ ID NO: 3, and an antisense strand comprising a nucleotide sequence differing by no more than 4 nucleotides from SEQ ID NO: 13. In some embodiments, provided herein is a RIZ 2 inhibitor comprising a double stranded polynucleotide comprising a sense strand of SEQ ID NO: 3, and an antisense strand of SEQ ID NO: 13.

In some embodiments, provided herein is a RIZ 2 inhibitor comprising a double stranded polynucleotide comprising a sense strand comprising a nucleotide sequence differing by no more than 4 nucleotides from SEQ ID NO: 4, and an antisense strand comprising a nucleotide sequence differing by no more than 4 nucleotides from SEQ ID NO: 14. In some embodiments, provided herein is a RIZ 2 inhibitor comprising a double stranded polynucleotide comprising a sense strand of SEQ ID NO: 4, and an antisense strand of SEQ ID NO: 14.

In some embodiments, provided herein is a RIZ 2 inhibitor comprising a double stranded polynucleotide comprising a sense strand comprising a nucleotide sequence differing by no more than 4 nucleotides from SEQ ID NO: 5, and an antisense strand comprising a nucleotide sequence differing by no more than 4 nucleotides from SEQ ID NO: 15. In some embodiments, provided herein is a RIZ 2 inhibitor comprising a double stranded polynucleotide comprising a sense strand of SEQ ID NO: 5, and an antisense strand of SEQ ID NO: 15.

In some embodiments, provided herein is a RIZ 2 inhibitor comprising a double stranded polynucleotide comprising a sense strand comprising a nucleotide sequence differing by no more than 4 nucleotides from SEQ ID NO: 7, and an antisense strand comprising a nucleotide sequence differing by no more than 4 nucleotides from SEQ ID NO: 16. In some embodiments, provided herein is a RIZ 2 inhibitor comprising a double stranded polynucleotide comprising a sense strand of SEQ ID NO: 7, and an antisense strand of SEQ ID NO: 16.

In some embodiments, provided herein is a RIZ 2 inhibitor comprising a double stranded polynucleotide comprising a sense strand comprising a nucleotide sequence differing by no more than 4 nucleotides from SEQ ID NO: 8, and an antisense strand comprising a nucleotide sequence differing by no more than 4 nucleotides from SEQ ID NO: 17. In some embodiments, provided herein is a RIZ 2 inhibitor comprising a double stranded polynucleotide comprising a sense strand of SEQ ID NO: 8, and an antisense strand of SEQ ID NO: 17.

In some embodiments, provided herein is a RIZ 2 inhibitor comprising a double stranded polynucleotide comprising a sense strand comprising a nucleotide sequence differing by no more than 4 nucleotides from SEQ ID NO: 9, and an antisense strand comprising a nucleotide sequence differing by no more than 4 nucleotides from SEQ ID NO: 18. In some embodiments, provided herein is a RIZ 2 inhibitor comprising a double stranded polynucleotide comprising a sense strand of SEQ ID NO: 9, and an antisense strand of SEQ ID NO: 18.

The sense strand can be 15 to 49 nucleotides in length. The antisense strand can be 18 to 49 nucleotides in length. The sense and antisense strands can be either the same length or they can be different lengths. In some aspects, the sense and antisense strands are each independently 18 to 27 nucleotides in length. In some aspects, both the sense and antisense strands are each 21-26 nucleotides in length. In some aspects, the sense and antisense strands are each 21-24 nucleotides in length. In some aspects, the sense and antisense strands are each independently 19-21 nucleotides in length. In some aspects, the sense strand is about 19 nucleotides in length while the antisense strand is about 21 nucleotides in length. In some aspects, the sense strand is about 21 nucleotides in length while the antisense strand is about 23 nucleotides in length. In some aspects, a sense strand is 23 nucleotides in length and an antisense strand is 21 nucleotides in length. In some aspects, both the sense and antisense strands are each 21 nucleotides in length. In some aspects, the RNAi agent antisense strands are each 18, 19, 20, 21, 22, 23, 24, 25, 26, 27, 28, 29, or 30 nucleotides in length. In some embodiments, the RNAi agent sense strands are each 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The sense and antisense strands are annealed to form a duplex, and in some aspects, a double-stranded RNAi agent has a duplex length of about 15, 16, 17, 18, 19, 20, 21, 22, 23 or 24 nucleotides.

Examples of nucleotide sequences used in forming PRDM2/RIZ2 RNAi agents are provided in Table 2. Examples of RNAi agent duplexes, that include the sense strand and antisense strand sequences in Table 2.

In some aspects, the region of perfect, substantial, or partial complementarity between the sense strand and the antisense strand is 15-26 (e.g., 15, 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the PRDM2/RIZ2 RNAi agents described herein includes at least 15 consecutive nucleotides that have at least 85% identity to a core stretch sequence (also referred to herein as a “core stretch” or “core sequence”) of the same number of nucleotides in an PRDM2/RIZ2 mRNA. In some aspects, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the PRDM2/RIZ2 mRNA target. In some aspects, this sense strand core stretch is 15, 16, 17, 18, 19, 20, 21, 22, or 23 nucleotides in length. In some aspects, this sense strand core stretch is 17 nucleotides in length. In some aspects, this sense strand core stretch is 19 nucleotides in length.

An antisense strand of an PRDM2/RIZ2 RNAi agent described herein includes at least 15 consecutive nucleotides that have at least 85% complementarity to a core stretch of the same number of nucleotides in an PRDM2/RIZ2 mRNA and to a core stretch of the same number of nucleotides in the corresponding sense strand. In some aspects, an antisense strand core stretch is 100% (perfectly) complementary or at least about 85% (substantially) complementary to a nucleotide sequence (e.g., target sequence) of the same length present in the PRDM2/RIZ2 mRNA target. In some aspects, this antisense strand core stretch is 15, 16, 17, 18, 19, 20, 21, 22, or 23 nucleotides in length. In some aspects, this antisense strand core stretch is 19 nucleotides in length. In some aspects, this antisense strand core stretch is 17 nucleotides in length. A sense strand core stretch sequence can be the same length as a corresponding antisense core sequence or it can be a different length.

The PRDM2/RIZ2 RNAi agent sense and antisense strands anneal to form a duplex. A sense strand and an antisense strand of an PRDM2/RIZ2 RNAi agent can be partially, substantially, or fully complementary to each other. Within the complementary duplex region, the sense strand core stretch sequence is at least 85% complementary or 100% complementary to the antisense core stretch sequence. In some aspects, the sense strand core stretch sequence contains a sequence of at least 15, at least 16, at least 17, at least 18, at least 19, at least 20, at least 21, at least 22, at least 23, at least 24, or at least 25 nucleotides that is at least 85% or 100% complementary to a corresponding 15, 16, 17, 18, 19, 20, 21, 22, 23, 24, or 25 nucleotide sequence of the antisense strand core stretch sequence (i.e., the sense and antisense core stretch sequences of an PRDM2/RIZ2 RNAi agent have a region of at least 15, at least 16, at least 17, at least 18, at least 19, at least 20, at least 21, at least 22, at least 23, at least 24, or at least 25 nucleotides that is at least 85% base paired or 100% base paired.)

In some aspects, the antisense strand of an PRDM2/RIZ2 RNAi agent disclosed herein differs by no more than 0, 1, 2, or 3 nucleotides from any of the antisense strand sequences in Table 2. In some aspects, the sense strand of an PRDM2/RIZ2 RNAi agent disclosed herein differs by no more than 0, 1, 2, or 3 nucleotides from any of the sense strand sequences in Table 2.

In some aspects, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the PRDM2/RIZ2 mRNA. The sense strand additional nucleotides, if present, may or may not be identical to the corresponding sequence in the PRDM2/RIZ2 mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some aspects, both the sense strand and the antisense strand of an RNAi agent contain 3′ and 5′ extensions. In some aspects, one or more of the 3′ extension nucleotides of one strand base pairs with one or more 5′ extension nucleotides of the other strand. In other aspects, one or more of 3′ extension nucleotides of one strand do not base pair with one or more 5′ extension nucleotides of the other strand. In some aspects, an PRDM2/RIZ2 RNAi agent has an antisense strand having a 3′ extension and a sense strand having a 5′ extension. In some aspects, the extension nucleotide(s) are unpaired and form an overhang. As used herein, an “overhang” refers to a stretch of one or more unpaired nucleotides located at a terminal end of either the sense strand or the antisense strand that does not form part of the hybridized or duplexed portion of an RNAi agent disclosed herein.

In some aspects, an PRDM2/RIZ2 RNAi agent comprises an antisense strand having a 3′ extension of 1, 2, 3, 4, 5, or 6 nucleotides in length. In other aspects, an PRDM2/RIZ2 RNAi agent comprises an antisense strand having a 3′ extension of 1, 2, or 3 nucleotides in length. In some aspects, one or more of the antisense strand extension nucleotides comprise nucleotides that are complementary to the corresponding PRDM2/RIZ2 mRNA sequence. In some aspects, one or more of the antisense strand extension nucleotides comprise nucleotides that are not complementary to the corresponding PRDM2/RIZ2 mRNA sequence.

In some aspects, an PRDM2/RIZ2 RNAi agent comprises a sense strand having a 3′ extension of 1, 2, 3, 4, or 5 nucleotides in length. In some aspects, one or more of the sense strand extension nucleotides comprises adenosine, uracil, or thymidine nucleotides, AT dinucleotide, or nucleotides that correspond to or are the identical to nucleotides in the PRDM2/RIZ2 mRNA sequence. In some aspects, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some aspects, an PRDM2/RIZ2 RNAi agent comprises a sense strand having a 5′ extension of 1, 2, 3, 4, 5, or 6 nucleotides in length. In some aspects, one or more of the sense strand extension nucleotides comprise nucleotides that correspond to or are identical to nucleotides in the PRDM2/RIZ2 mRNA sequence.

Examples of sequences used in forming PRDM2/RIZ2 RNAi agents are provided in Table 2. In some aspects, an PRDM2/RIZ2 RNAi agent antisense strand includes a sequence of any of the sequences in Table 2 (e.g., any one of SEQ ID NOs: 10-18). The PRDM2/RIZ2 RNAi agent antisense strand may be modified or unmodified. In certain aspects, an PRDM2/RIZ2 RNAi agent antisense strand comprises or consists of any one of the modified sequences in Table 2. In some aspects, an PRDM2/RIZ2 RNAi agent antisense strand includes the sequence of nucleotides (from 5′ end→3′ end) at positions 1-17, 2-15, 2-17, 1-18, 2-18, 1-19, 2-19, 1-20, 2-20, 1-21, or 2-21, of any of the sequences in Table 2. In some aspects, an PRDM2/RIZ2 RNAi agent sense strand includes the sequence of any of the sequences in Table 2. In some aspects, an PRDM2/RIZ2 RNAi agent sense strand includes the sequence of nucleotides (from 5′ end→3′ end) at positions 1-18, 1-19, 1-20, 1-21, 2-19, 2-20, 2-21, 3-20, 3-21, or 4-21 of any of the sequences in Table 2. In certain aspects, an PRDM2/RIZ2 RNAi agent sense strand comprises or consists of a modified sequence of any one of the modified sequences in Table 2.

In some aspects, the sense and antisense strands of the RNAi agents described herein contain the same number of nucleotides. In some aspects, the sense and antisense strands of the RNAi agents described herein contain different numbers of nucleotides. In some aspects, the sense strand 5′ end and the antisense strand 3′ end of an RNAi agent form a blunt end. In some aspects, the sense strand 3′ end and the antisense strand 5′ end of an RNAi agent form a blunt end. In some aspects, both ends of an RNAi agent form blunt ends. In some aspects, neither end of an RNAi agent is blunt-ended. As used herein a “blunt end” refers to an end of a double stranded RNAi agent in which the terminal nucleotides of the two annealed strands are complementary (form a complementary base-pair).

In some aspects, the sense strand 5′ end and the antisense strand 3′ end of an RNAi agent form a frayed end. In some aspects, the sense strand 3′ end and the antisense strand 5′ end of an RNAi agent form a frayed end. In some aspects, both ends of an RNAi agent form a frayed end. In some aspects, neither end of an RNAi agent is a frayed end. As used herein a frayed end refers to an end of a double stranded RNAi agent in which the terminal nucleotides of the two annealed strands from a pair (i.e., do not form an overhang) but are not complementary (i.e. form a non-complementary pair). In some aspects, one or more unpaired nucleotides at the end of one strand of a double stranded RNAi agent form an overhang. The unpaired nucleotides may be on the sense strand or the antisense strand, creating either 3′ or 5′ overhangs. In some aspects, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The PRDM2/RIZ2 RNAi agents disclosed herein may also be comprised of one or more modified nucleotides. In some aspects, substantially all of the nucleotides of the sense strand and substantially all of the nucleotides of the antisense strand of the PRDM2/RIZ2 RNAi agent are modified nucleotides. In some embodiments, the one or more modified nucleotides is a 2′-modified nucleotide (e.g., 2′-methoxyuridine or 2′-fluoro-deoxyuridine). The PRDM2/RIZ2 RNAi agents disclosed herein may further be comprised of one or more modified internucleoside linkages, e.g., one or more phosphorothioate linkages. In some aspects, an PRDM2/RIZ2 RNAi agent contains one or more modified nucleotides and one or more modified internucleoside linkages. In some aspects, a 2′-modified nucleotide is combined with modified internucleoside linkage.

In some aspects, an PRDM2/RIZ2 RNAi agent is prepared or provided as a salt, mixed salt, or a free-acid. In some aspects, an PRDM2/RIZ2 RNAi agent is prepared as a pharmaceutically acceptable salt. In some aspects, an PRDM2/RIZ2 RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administering of the oligonucleotide construct.

In some aspects, an PRDM2/RIZ2 RNAi agent contains one or more modified nucleotides. As used herein, a “modified nucleotide” is a nucleotide other than a ribonucleotide (2′-hydroxyl nucleotide). In some aspects, a modified nucleotide comprises a nucleobase modification, a ribose modification, a backbone modification, or combinations thereof. In some aspects, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e., a nucleotide with a group other than a hydroxyl group at the 2′ position of the five-membered sugar ring) include, but are not limited to, 2′-O-methyl nucleotides, 2′-fluoro nucleotides (also referred to herein as 2′-deoxy-2′-fluoro nucleotides), 2′-deoxy nucleotides, 2′-methoxyethyl (2′-O-2-methoxylethyl) nucleotides (also referred to as 2′-MOE), 2′-amino nucleotides, and 2′-alkyl nucleotides. It is not necessary for all positions in a given compound to be uniformly modified. Conversely, more than one modification can be incorporated in a single PRDM2/RIZ2 RNAi agent or even in a single nucleotide thereof. The PRDM2/RIZ2 RNAi agent sense strands and antisense strands can be synthesized and/or modified by methods known in the art. Modification at one nucleotide is independent of modification at another nucleotide.

In some embodiments, the PRDM2/RIZ2 RNAi agent comprises one more modified nucleobase. Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some aspects, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. In some aspects, an abasic residue can be placed internally in a nucleotide sequence. In some aspects, Ab or AbAb can be added to the 3′ end of the antisense strand. In some aspects, the 5′ end of the sense strand can include one or more additional abasic residues (e.g., (Ab) or (AbAb)). In some aspects, UUAb, UAb, or Ab are added to the 3′ end of the sense strand. In some aspects, an abasic (deoxyribose) residue can be replaced with a ribitol (abasic ribose) residue.

In some aspects, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the sense strand being unmodified ribonucleotides. In some aspects, one or more nucleotides of an RNAi agent is an unmodified ribonucleotide.

### Modified Internucleoside Linkages

In some aspects, one or more nucleotides of an PRDM2/RIZ2 RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some aspects, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some aspects, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some aspects, a sense strand of an PRDM2/RIZ2 RNAi agent can contain 1, 2, 3, 4, 5, or 6 phosphorothioate linkages, an antisense strand of an PRDM2/RIZ2 RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some aspects, a sense strand of an PRDM2/RIZ2 RNAi agent can contain 1, 2, 3, or 4 phosphorothioate linkages, an antisense strand of an PRDM2/RIZ2 RNAi agent can contain 1, 2, 3, or 4 phosphorothioate linkages, or both the sense strand and the antisense strand independently can contain 1, 2, 3, or 4 phosphorothioate linkages.

In some aspects, an PRDM2/RIZ2 RNAi agent sense strand contains at least two phosphorothioate internucleoside linkages. In some aspects, the phosphorothioate internucleoside linkages are between the nucleotides at positions 1-3 from the 3′ end of the sense strand. In some aspects, one phosphorothioate internucleoside linkage is at the 5′ end of the sense strand nucleotide sequence, and another phosphorothioate linkage is at the 3′ end of the sense strand nucleotide sequence. In some aspects, two phosphorothioate internucleoside linkages are located at the 5′ end of the sense strand, and another phosphorothioate linkage is at the 3′ end of the sense strand. In some aspects, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some aspects, the targeting ligand is linked to the sense strand via a phosphorothioate linkage.

In some aspects, an PRDM2/RIZ2 RNAi agent antisense strand contains four phosphorothioate internucleoside linkages. In some aspects, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some aspects, three phosphorothioate internucleoside linkages are located between positions 1-4 from the 5′ end of the antisense strand, and a fourth phosphorothioate internucleoside linkage is located between positions 20-21 from the 5′ end of the antisense strand. In some aspects, an PRDM2/RIZ2 RNAi agent contains at least three or four phosphorothioate internucleoside linkages in the antisense strand.

### Capping Residues or Moieties

In some aspects, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some aspects, inverted abasic residues (invAb) (also referred to in the art as “inverted abasic sites”) are added as capping residues. (See, e.g., F. Czauderna, Nucleic Acids Res., 2003, 31(11), 2705-16; U.S. Pat. No. 5,998,203). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some aspects, a capping residue is present at either the 5′ terminal end, the 3′ terminal end, or both the 5′ and 3′ terminal ends of the sense strand. In some aspects, the 5′ end and/or the 3′ end of the sense strand may include more than one inverted abasic deoxyribose moiety as a capping residue.

In some aspects, one or more inverted abasic residues (invAb) are added to the 3′ end of the sense strand. In some aspects, one or more inverted abasic residues (invAb) are added to the 5′ end of the sense strand. In some aspects, one or more inverted abasic residues or inverted abasic sites are inserted between the targeting ligand and the nucleotide sequence of the sense strand of the RNAi agent. In some aspects, the inclusion of one or more inverted abasic residues or inverted abasic sites at or near the terminal end or terminal ends of the sense strand of an RNAi agent allows for enhanced activity or other desired properties of an RNAi agent.

In some aspects, one or more inverted abasic residues (invAb) are added to the 5′ end of the sense strand. In some aspects, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb)s)), or other linkages. In some aspects, the inclusion of one or more inverted abasic residues at or near the terminal end or terminal ends of the sense strand of an RNAi agent may allow for enhanced activity or other desired properties of an RNAi agent. In some aspects, an inverted abasic (deoxyribose) residue can be replaced with an inverted ribitol (abasic ribose) residue. In some aspects, the 3′ end of the antisense strand core stretch sequence, or the 3′ end of the antisense strand sequence, may include an inverted abasic residue.

In one aspect, the present invention provides double stranded polynucleotide compositions for inhibiting RIZ2 mRNA expression. In some embodiments, the double stranded polynucleotide may occur without any modifications. In some embodiments, one or more nucleotides within the sense or antisense molecule is modified. For example ARIZ-047 sense and antisense strands have the same nucleotide sequence as ARIZ-011 but comprises modified nucleotides, e.g., the sense strand comprises a disulfide modification at the 5′-end. In some embodiments, the oligomer comprises one modified nucleotide, e.g., a 2′-methoxyuridine interrupting the stretch of non-modified nucleotides, e.g., the 9^{th} nucleotide residue from the 5′-end of SEQ ID NO: 6. In some embodiments, the sense or the antisense strand may comprise more than one modifications, For example, the antisense strand of ARIZ-047 (SEQ ID NO: 11) comprises two 5′-P′-methoxyuridine residues in tandem, and a terminal 3′-dinucleotide at the 3′end comprise phosphorothioate bonds.

Contemplated herein are one or more modifications to either the sense strand, or the antisense strand for any one of the double stranded polynucleotide compositions presented herein in table 1. In some embodiments, one or both the strands are designed to comprise a modification. In some embodiments, one strand may comprise an interrupted or gapped motif and the other strand may comprise a gapped motif, a hemimer motif, a blockmer motif, a fully modified motif, a positionally modified motif or an alternating motif. An “interrupted” or “gapped” motif comprises a modified nucleoside interrupting a contiguous sequence of nucleosides, such that the nucleotide stretch is divided into 2, or preferably 3 regions, e.g., an internal region flanked by two external regions. The regions are interrupted and separated from each other at least by having modified (different) sugar groups that comprise the nucleosides. In some embodiments, the nucleosides with different sugar groups comprise oligomeric compound p-D-ribonucleosides, or a 2′ modified nucleosides, or a 4′-thio modified nucleosides, or a 4′-thio-2′-modified nucleosides, or a bicyclic sugar modified nucleosides.

In some embodiments, the internal region or the gap generally comprises p-D-ribonucleosides but can be a sequence of sugar modified nucleosides. In some embodiments, the nucleosides located in the gap of a gapped oligomeric compound have different sugar groups than both of the wings. In some embodiments, the gapped oligomeric compounds are “symmetric”. In some embodiments, the gapped oligomeric compounds are “asymmetric”. A gapmer having the same uniform sugar modification in each of the wings may be a symmetric gapped oligomeric compound. A gapmer having different uniform modifications in each wing is termed an asymmetric gapped oligomeric compound. In some embodiments, the gapped oligomeric compounds such as these can have for example both wings comprising 4′-thio modified nucleosides (symmetric gapmer) and a gap comprising p-D-ribonucleosides or modified nucleosides other than 4′-thio modified nucleosides. In some embodiments, the asymmetric gapped oligomeric compounds may comprise one wing comprising 2′-OCH 3 modified nucleosides; and the other wing comprising 4′-thio modified nucleosides with the internal region (gap) comprising p D-ribonucleosides or sugar modified nucleosides that are other than 4′-thio or 2′-OCH 3 modified nucleosides.

In some embodiments, each strand of the compositions of the present invention can be modified to fulfil a particular role in for example the siRNA pathway. The antisense strand can be modified at the 5′-end to enhance its role in one region of the RISC while 5 the 3′-end can be modified differentially to enhance its role in a different region of the RISC.

In some embodiments, siRNAs comprise a T overhang. In some embodiments, the T overhang comprises a single Uridine (interchangeably designated as Thymidine (T) in the sequences presented herein, or two Uridine nucleotide residues in tandem (e.g., dTdT).

In some embodiments, one or more residues are linked by phosphorothioate bonds.

In one aspect, provided herein is a use of any one of the RIZ2 siRNAs described in the specification for treatment of a hyperproliferative disorder, e.g., cancer. In one aspect, provided herein is a use of any one of the RIZ2 siRNAs described in the specification for preparing a medicament suitable for the treatment of a hyperproliferative disorder, e.g., cancer.

siRNAs ARIZ-011 through -014 are complementary to sequences shared by both the RIZ1 and RIZ2 mRNA transcripts. ARIZ-015 is complementary to the last 19 bases at the 3′ end of the RIZ2 mRNA, and contains 10 nucleotides unique to RIZ2 mRNA that correspond to four terminal amino acids unique to RIZ2, as well as 9 nucleotides common to both RIZ1 and RIZ2 mRNA. ARIZ-047 is the same sequence as ARIZ-011 except for the indicated base modifications.

siRNAs ARIZ-062 through -064 are complementary to sequences unique to RIZ2 mRNA.

In some embodiments, provided herein is an siRNA that targets only RIZ2 and spares RIZ1 mRNA. In some embodiments, provided herein is an siRNA that targets only RIZ1 but does not affect RIZ2 mRNA. In some embodiments, provided herein is an siRNA that targets RIZ2 mRNA, and spares RIZ1 mRNA, and that exerts an inhibitory effect on RIZ2 mRNA. Provided herein are surprising results that, despite the fact that an siRNA disclosed herein can target either of RIZ1 or RIZ2 mRNA, Applicant's observations indicate that the siRNA specifically inhibits RIZ2 mRNA. Yet more surprising was the observation, as presented herein, that the siRNA results in increase of RIZ1 mRNA level. Even further surprising are the observations disclosed for the first time herein, is that the siRNA of the invention can preferentially lead to cell death of cancer cells and spares the non-cancer cells. This is highly unexpected in a condition where the siRNA can target a region common to both RIZ2 and RIZ1 mRNA and therefore potentially inhibit both mRNAs.

In some embodiments, the siRNA provided herein targets a RIZ2 mRNA, which when present at a higher concentration in a given cell, e.g., a diseased cell, a cell with hyperproliferative disorder such as a cancer cell is available to manipulation by the siRNA, compared to the concurrent low concentration of the RIZ1 mRNA as occurs in a diseased cell, e.g., a cancer cell. Without wishing to be bound by any theory, it may be possible that once the siRNA of the invention targets a RIZ2 mRNA which is more prone to be targeted being at a higher concentration than RIZ1 mRNA in a diseased cell, the siRNA inhibits RIZ2 expression, leading to a concomitant increase in RIZ1 mRNA. Increase in RIZ1 mRNA can help reset the cell cycle homeostasis and the cell death may be achieved for the aberrantly proliferative cells, e.g., cancer cells.

In some embodiments, the RIZ2 inhibitor comprising any one of the siRNA in Table 2 reduces the expression of RIZ2 mRNA in a treated cell by at least 5% compared to an untreated cell. In some embodiments, a RIZ2 siRNA reduces the expression of RIZ2 mRNA by at least 6%. In some embodiments, a RIZ2 siRNA reduces the expression of RIZ2 mRNA by at least 7%. In some embodiments, a RIZ2 siRNA reduces the expression of RIZ2 mRNA by at least 8%. In some embodiments, a RIZ2 siRNA reduces the expression of RIZ2 mRNA by at least 9%. In some embodiments, a RIZ2 siRNA reduces the expression of RIZ2 mRNA by at least 10%. In some embodiments, a RIZ2 siRNA reduces the expression of RIZ2 mRNA by at least 11%. In some embodiments, a RIZ2 siRNA reduces the expression of RIZ2 mRNA by at least 12%. In some embodiments, a RIZ2 siRNA reduces the expression of RIZ2 mRNA by at least 13%. In some embodiments, a RIZ2 siRNA reduces the expression of RIZ2 mRNA by at least 14%. In some embodiments, a RIZ2 siRNA reduces the expression of RIZ2 mRNA by at least 15%. In some embodiments, a RIZ2 siRNA reduces the expression of RIZ2 mRNA by at least 16%. In some embodiments, a RIZ2 siRNA reduces the expression of RIZ2 mRNA by at least 17%. In some embodiments, a RIZ2 siRNA reduces the expression of RIZ2 mRNA by at least 18%. In some embodiments, a RIZ2 siRNA reduces the expression of RIZ2 mRNA by at least 19%. In some embodiments, a RIZ2 siRNA reduces the expression of RIZ2 mRNA by at least 20%. In some embodiments, a RIZ2 siRNA reduces the expression of RIZ2 mRNA by at least 21%. In some embodiments, a RIZ2 siRNA reduces the expression of RIZ2 mRNA by at least 22%. In some embodiments, a RIZ2 siRNA reduces the expression of RIZ2 mRNA by at least 23%. In some embodiments, a RIZ2 siRNA reduces the expression of RIZ2 mRNA by at least 24%. In some embodiments, a RIZ2 siRNA reduces the expression of RIZ2 mRNA by at least 25%. In some embodiments, a RIZ2 siRNA reduces the expression of RIZ2 mRNA by at least 26%. In some embodiments, a RIZ2 siRNA reduces the expression of RIZ2 mRNA by at least 27%. In some embodiments, a RIZ2 siRNA reduces the expression of RIZ2 mRNA by at least 28%. In some embodiments, a RIZ2 siRNA reduces the expression of RIZ2 mRNA by at least 29%. In some embodiments, a RIZ2 siRNA reduces the expression of RIZ2 mRNA by at least 30%. In some embodiments, a RIZ2 siRNA reduces the expression of RIZ2 mRNA by at least 31%. In some embodiments, a RIZ2 siRNA reduces the expression of RIZ2 mRNA by at least 32%. In some embodiments, a RIZ2 siRNA reduces the expression of RIZ2 mRNA by at least 33%. In some embodiments, a RIZ2 siRNA reduces the expression of RIZ2 mRNA by at least 34%. In some embodiments, a RIZ2 siRNA reduces the expression of RIZ2 mRNA by at least 35%. In some embodiments, a RIZ2 siRNA reduces the expression of RIZ2 mRNA by at least 36%. In some embodiments, a RIZ2 siRNA reduces the expression of RIZ2 mRNA by at least 37%. In some embodiments, a RIZ2 siRNA reduces the expression of RIZ2 mRNA by at least 38%. In some embodiments, a RIZ2 siRNA reduces the expression of RIZ2 mRNA by at least 39%. In some embodiments, a RIZ2 siRNA reduces the expression of RIZ2 mRNA by at least 40%. In some embodiments, a RIZ2 siRNA reduces the expression of RIZ2 mRNA by at least 41%. In some embodiments, a RIZ2 siRNA reduces the expression of RIZ2 mRNA by at least 42%. In some embodiments, a RIZ2 siRNA reduces the expression of RIZ2 mRNA by at least 43%. In some embodiments, a RIZ2 siRNA reduces the expression of RIZ2 mRNA by at least 44%. In some embodiments, a RIZ2 siRNA reduces the expression of RIZ2 mRNA by at least 45%. In some embodiments, a RIZ2 siRNA reduces the expression of RIZ2 mRNA by at least 46%. In some embodiments, a RIZ2 siRNA reduces the expression of RIZ2 mRNA by at least 47%. In some embodiments, a RIZ2 siRNA reduces the expression of RIZ2 mRNA by at least 48%. In some embodiments, a RIZ2 siRNA reduces the expression of RIZ2 mRNA by at least 49%. In some embodiments, a RIZ2 siRNA reduces the expression of RIZ2 mRNA by at least 50%. In some embodiments, a RIZ2 siRNA reduces the expression of RIZ2 mRNA by at least 51%. In some embodiments, a RIZ2 siRNA reduces the expression of RIZ2 mRNA by at least 52%. In some embodiments, a RIZ2 siRNA reduces the expression of RIZ2 mRNA by at least 53%. In some embodiments, a RIZ2 siRNA reduces the expression of RIZ2 mRNA by at least 54%. In some embodiments, a RIZ2 siRNA reduces the expression of RIZ2 mRNA by at least 55%. In some embodiments, a RIZ2 siRNA reduces the expression of RIZ2 mRNA by at least 56%. In some embodiments, a RIZ2 siRNA reduces the expression of RIZ2 mRNA by at least 57%. In some embodiments, a RIZ2 siRNA reduces the expression of RIZ2 mRNA by at least 58%. In some embodiments, a RIZ2 siRNA reduces the expression of RIZ2 mRNA by at least 59%. In some embodiments, a RIZ2 siRNA reduces the expression of RIZ2 mRNA by at least 60%. In some embodiments, a RIZ2 siRNA reduces the expression of RIZ2 mRNA by at least 61%. In some embodiments, a RIZ2 siRNA reduces the expression of RIZ2 mRNA by at least 62%. In some embodiments, a RIZ2 siRNA reduces the expression of RIZ2 mRNA by at least 63%. In some embodiments, a RIZ2 siRNA reduces the expression of RIZ2 mRNA by at least 64%. In some embodiments, a RIZ2 siRNA reduces the expression of RIZ2 mRNA by at least 65%. In some embodiments, a RIZ2 siRNA reduces the expression of RIZ2 mRNA by at least 66%. In some embodiments, a RIZ2 siRNA reduces the expression of RIZ2 mRNA by at least 67%. In some embodiments, a RIZ2 siRNA reduces the expression of RIZ2 mRNA by at least 68%. In some embodiments, a RIZ2 siRNA reduces the expression of RIZ2 mRNA by at least 69%. In some embodiments, a RIZ2 siRNA reduces the expression of RIZ2 mRNA by at least 70%. In some embodiments, a RIZ2 siRNA reduces the expression of RIZ2 mRNA by at least 71%. In some embodiments, a RIZ2 siRNA reduces the expression of RIZ2 mRNA by at least 72%. In some embodiments, a RIZ2 siRNA reduces the expression of RIZ2 mRNA by at least 73%. In some embodiments, a RIZ2 siRNA reduces the expression of RIZ2 mRNA by at least 74%. In some embodiments, a RIZ2 siRNA reduces the expression of RIZ2 mRNA by at least 75%. In some embodiments, a RIZ2 siRNA reduces the expression of RIZ2 mRNA by at least 76%. In some embodiments, a RIZ2 siRNA reduces the expression of RIZ2 mRNA by at least 77%. In some embodiments, a RIZ2 siRNA reduces the expression of RIZ2 mRNA by at least 78%. In some embodiments, a RIZ2 siRNA reduces the expression of RIZ2 mRNA by at least 79%. In some embodiments, a RIZ2 siRNA reduces the expression of RIZ2 mRNA by at least 80%. In some embodiments, a RIZ2 siRNA reduces the expression of RIZ2 mRNA by at least 81%. In some embodiments, a RIZ2 siRNA reduces the expression of RIZ2 mRNA by at least 82%. In some embodiments, a RIZ2 siRNA reduces the expression of RIZ2 mRNA by at least 83%. In some embodiments, a RIZ2 siRNA reduces the expression of RIZ2 mRNA by at least 84%. In some embodiments, a RIZ2 siRNA reduces the expression of RIZ2 mRNA by at least 85%. In some embodiments, a RIZ2 siRNA reduces the expression of RIZ2 mRNA by at least 86%. In some embodiments, a RIZ2 siRNA reduces the expression of RIZ2 mRNA by at least 87%. In some embodiments, a RIZ2 siRNA reduces the expression of RIZ2 mRNA by at least 88%. In some embodiments, a RIZ2 siRNA reduces the expression of RIZ2 mRNA by at least 89%. In some embodiments, a RIZ2 siRNA reduces the expression of RIZ2 mRNA by at least 90%.

In some embodiments, a RIZ2 siRNA reduces the expression of RIZ2 mRNA as compared to expression of a housekeeping gene in the cells. exemplary housekeeping genes are GAPDH, or beta actin gene. In some embodiments, a RIZ2 siRNA reduces the expression of RIZ2 mRNA as compared to the expression of RIZ2 mRNA in a control cell or a population of control cells. Exemplary housekeeping genes are GAPDH, or beta actin gene.

In some embodiments, provided herein is a method of restoring RIZ1-RIZ2 balance in a cell. In some embodiments, the ratio of RIZ1 to RIZ2 mRNA expression levels are altered by greater than at least 1.1-fold. For example, in some embodiments, the ratio of RIZ1 to RIZ2 mRNA expression levels are altered by greater than at least 1.2-fold. In some embodiments, the ratio of RIZ1 to RIZ2 mRNA expression levels are altered by greater than at least 1.3-fold. In some embodiments, the ratio of RIZ1 to RIZ2 mRNA expression levels are altered by greater than at least 1.4-fold. In some embodiments, the ratio of RIZ1 to RIZ2 mRNA expression levels are altered by greater than at least 1.5-fold. In some embodiments, the ratio of RIZ1 to RIZ2 mRNA expression levels are altered by greater than at least 1.6-fold. In some embodiments, the ratio of RIZ1 to RIZ2 mRNA expression levels are altered by greater than at least 1.7-fold. In some embodiments, the ratio of RIZ1 to RIZ2 mRNA expression levels are altered by greater than at least 1.8-fold. In some embodiments, the ratio of RIZ1 to RIZ2 mRNA expression levels are altered by greater than at least 1.9-fold. In some embodiments, the ratio of RIZ1 to RIZ2 mRNA expression levels are alters the ratio of RIZ1 to RIZ2 mRNA expression levels by at least 2-fold.

In some embodiments, the RIZ2 inhibitor is an siRNA that reduces cell proliferation of a diseased cell (e.g. cancer cell) by at least 10%. In some embodiments the RIZ2 inhibitor siRNA reduces cancer cell proliferation by about 15%. In some embodiments the RIZ2 inhibitor siRNA reduces cancer cell proliferation by about 20%. In some embodiments the RIZ2 inhibitor siRNA reduces cancer cell proliferation by about 25%. In some embodiments the RIZ2 inhibitor siRNA reduces cancer cell proliferation by about 30%. In some embodiments the RIZ2 inhibitor siRNA reduces cancer cell proliferation by about 35%. In some embodiments the RIZ2 inhibitor siRNA reduces cancer cell proliferation by about 40%. In some embodiments the RIZ2 inhibitor siRNA reduces cancer cell proliferation by about 45%. In some embodiments the RIZ2 inhibitor siRNA reduces cancer cell proliferation by about 50%. In some embodiments the RIZ2 inhibitor siRNA reduces cancer cell proliferation by about 55%. In some embodiments the RIZ2 inhibitor siRNA reduces cancer cell proliferation by about 60%. In some embodiments the RIZ2 inhibitor siRNA reduces cancer cell proliferation by about 65%. In some embodiments the RIZ2 inhibitor siRNA reduces cancer cell proliferation by about 70%. In some embodiments the RIZ2 inhibitor siRNA reduces cancer cell proliferation by about 75%. In some embodiments the RIZ2 inhibitor siRNA reduces cancer cell proliferation by about 80%. In some embodiments the RIZ2 inhibitor siRNA reduces cancer cell proliferation by about 81%. In some embodiments the RIZ2 inhibitor siRNA reduces cancer cell proliferation by about 82%. In some embodiments the RIZ2 inhibitor siRNA reduces cancer cell proliferation by about 83%. In some embodiments the RIZ2 inhibitor siRNA reduces cancer cell proliferation by about 84%. In some embodiments the RIZ2 inhibitor siRNA reduces cancer cell proliferation by about 85%. In some embodiments the RIZ2 inhibitor siRNA reduces cancer cell proliferation by about 86%. In some embodiments the RIZ2 inhibitor siRNA reduces cancer cell proliferation by about 87%. In some embodiments the RIZ2 inhibitor siRNA reduces cancer cell proliferation by about 88%. In some embodiments the RIZ2 inhibitor siRNA reduces cancer cell proliferation by about 89%. In some embodiments the RIZ2 inhibitor siRNA reduces cancer cell proliferation by about 90%. In some embodiments the RIZ2 inhibitor siRNA reduces cancer cell proliferation by about 91%. In some embodiments the RIZ2 inhibitor siRNA reduces cancer cell proliferation by about 92%. In some embodiments the RIZ2 inhibitor siRNA reduces cancer cell proliferation by about 93%. In some embodiments the RIZ2 inhibitor siRNA reduces cancer cell proliferation by about 94%. In some embodiments the RIZ2 inhibitor siRNA reduces cancer cell proliferation by about 95%. In some embodiments the RIZ2 inhibitor siRNA reduces cancer cell proliferation by about 96%. In some embodiments the RIZ2 inhibitor siRNA reduces cancer cell proliferation by about 97%. In some embodiments the RIZ2 inhibitor siRNA reduces cancer cell proliferation by about 98%.

In some embodiments the RIZ2 inhibitor siRNA reduces cancer cell proliferation by about 35%. In some embodiments the RIZ2 inhibitor siRNA reduces cancer cell proliferation by about 40%. In some embodiments the RIZ2 inhibitor siRNA reduces cancer cell proliferation by about 45%. In some embodiments the RIZ2 inhibitor siRNA reduces cancer cell proliferation by about 50%. In some embodiments the RIZ2 inhibitor siRNA reduces cancer cell proliferation by about 55%. In some embodiments the RIZ2 inhibitor siRNA reduces cancer cell proliferation by about 60%. In some embodiments the RIZ2 inhibitor siRNA reduces cancer cell proliferation by about 65%. In some embodiments the RIZ2 inhibitor siRNA kills cancer cells by about 70%. In some embodiments the RIZ2 inhibitor siRNA kills cancer cells by about 75%. In some embodiments the RIZ2 inhibitor siRNA kills cancer cells by about 80%. In some embodiments the RIZ2 inhibitor siRNA kills cancer cells by about 81%. In some embodiments the RIZ2 inhibitor siRNA kills cancer cells by about 82%. In some embodiments the RIZ2 inhibitor siRNA kills cancer cells by about 83%. In some embodiments the RIZ2 inhibitor siRNA kills cancer cells by about 84%. In some embodiments the RIZ2 inhibitor siRNA kills cancer cells by about 85%. In some embodiments the RIZ2 inhibitor siRNA kills cancer cells by about 86%. In some embodiments the RIZ2 inhibitor siRNA kills cancer cells by about 87%. In some embodiments the RIZ2 inhibitor siRNA kills cancer cells by about 88%. In some embodiments the RIZ2 inhibitor siRNA kills cancer cells by about 89%. In some embodiments the RIZ2 inhibitor siRNA kills cancer cells by about 90%. In some embodiments the RIZ2 inhibitor siRNA kills cancer cells by about 91%. In some embodiments the RIZ2 inhibitor siRNA kills cancer cells by about 92%. In some embodiments the RIZ2 inhibitor siRNA kills cancer cells by about 93%. In some embodiments the RIZ2 inhibitor siRNA kills cancer cells by about 94%. In some embodiments the RIZ2 inhibitor siRNA kills cancer cells by about 95%. In some embodiments the RIZ2 inhibitor siRNA kills cancer cells by about 96%. In some embodiments the RIZ2 inhibitor siRNA kills cancer cells by about 97%. In some embodiments the RIZ2 inhibitor siRNA kills cancer cells by about 98%.

In one embodiment the RIZ2 inhibitor siRNA may reduce tumor cell mass by at least about 10%. In one embodiment the RIZ2 inhibitor siRNA may reduce cell mass (e.g. tumor cell mass) by about 50% compared to the untreated cells, or compared to a control cell population. In some embodiments, the reduction in cell mass may be greater than 50%, e.g., by about 55%, 60%, 65%, 70%, 75%, 80%, 85%, or 90%.

In some embodiments, the RIZ2 inhibitor siRNA does not significantly affect a non-cancer cell. In some embodiments, the RIZ2 inhibitor siRNA kills cancer cells by about 2 fold, 2.2 fold.

In some embodiments, one or more siRNAs are formulated into pharmaceutical compositions for use in treating a hyperproliferative disorder, or a neoplasia, e.g., breast cancer, colon cancer, endometrial cancer, esophageal cancer, glioma, kidney cancer, leukemia, lymphoma, lung cancer, liver cancer, parathyroid cancer, pituitary cancer, meningiomas, myeloma, neuroblastoma, prostate cancer, or thyroid cancers.

siRNA Delivery

In one aspect, the siRNA of the invention is encapsulated in a liposome.

In another aspect, the siRNA of the invention is a naked double stranded molecule.

In some embodiments, the siRNA of the invention is conjugated to a cell targeting moiety. In some embodiments, the siRNA of the invention is associated with a cell targeting moiety. In some embodiments, the siRNA is associated with a cell targeting moiety that is capable of targeting a cell surface element. In an exemplary embodiment, the cell targeting moiety targets a cancer cell, or a hyperproliferative cell.

In some embodiments, the cell targeting moiety is a ligand, and antibody or an antibody fragment, a single chain antibody, a peptide or an aptamer. In some embodiments, the cell targeting moiety is a cell-penetrating peptide. In some embodiments, the delivery system comprises one or more of lipids, cyclodextrin, chitosan, carbohydrate polymers, elastin-like polymers (ELP), calcium phosphate polymers or combinations thereof.

In some embodiments, the delivery system comprises cyclodextrin.

In some embodiments, the delivery system comprises chitosan.

In some embodiments, the delivery system comprises carbohydrate polymers.

In some embodiments, the delivery system comprises elastin-like polymers (ELP). In some embodiments, the targeted delivery system comprises passively targeted nanocarriers. “Passively” targeted nanocarriers utilize the enhanced permeability and retention (EPR) effect.

In some embodiments, specific molecules or ligands on cancer cell surface are targeted, with e.g., an antibody or a fragment thereof. For example, single chain anti-prostate stem cell antigen (PSCA) antibody (scAb_{PSCA}) as a specific ‘address tag’ for prostate cancer targeted imaging and therapy (Ling Y, Wei K, Luo Y, Gao X, Zhong S. Dual docetaxel/superparamagnetic iron oxide loaded nanoparticles for both targeting magnetic resonance imaging and cancer therapy. Biomaterials. 2011; 32:7139-50). For example, Hadjipanayis et al. employed an anti-epidermal growth factor receptor (EGFR) deletion mutant antibody to fabricate iron oxide nanoparticles for targeted imaging and therapeutic treatment of glioblastoma (Hadjipanayis CG, Machaidze R, Kaluzova M, Wang L, Schuette A J, Chen H. et al. EGFRvIII antibody-conjugated iron oxide nanoparticles for magnetic resonance imaging-guided convection-enhanced delivery and targeted therapy of glioblastoma. Cancer Res. 2010; 70:6303-12). For example, Chen and Shuai et al. used a CD3 single chain antibody (scAb_{CD3}) functionalized nonviral polymeric vector for gene delivery to T cells (Chen G, Chen W, Wu Z, Yuan R, Li H, Gao J. et al. MRI-visible polymeric vector bearing CD3 single chain antibody for gene delivery to T cells for immunosuppression. Biomaterials. 2009; 30:1962-70). Any of these techniques can be adapted into the siRNA delivery mechanisms contemplated herein.

In some embodiments, the siRNA is associated with one or more lipid components, e.g., a cationic lipid. In some embodiments, the siRNA is associated with one or more lipid components, e.g., at least a cationic lipid and an anionic lipid. In some embodiments, the siRNA is modified with a polyethylene glycol (PEG) molecule. In some embodiments, the siRNA is pegylated with a PEG. In some embodiments, the siRNA is associated in a complex comprising one or more lipids and a PEG. In some embodiments, the siRNA is associated in a complex comprising one or more lipids and a PEG wherein the PEG has a fixed length. In some embodiments, the PEG comprises a chain having about 20-about 120 carbon atoms. In some embodiments, the PEG comprises a chain having about 40-120 carbon atoms. In some embodiments, the PEG comprises a chain having about 60-120 carbon atoms. In some embodiments, the PEG comprises a chain having about 80-120 carbon atoms. In some embodiments, the PEG comprises a chain having about 100-120 carbon atoms. In some embodiments, the PEG comprises a chain having about 20-40 carbon atoms. In some embodiments, the PEG comprises a chain having about 20-60 carbon atoms. In some embodiments, the PEG comprises a chain having about 20-80 carbon atoms. In some embodiments, the PEG comprises a chain having about 20-100 carbon atoms. In some embodiments, the PEG comprises a chain having about 20-80 carbon atoms. In some embodiments, the PEG comprises a chain having about 20-1000 carbon atoms. In some embodiments, the PEG comprises a chain having about 20-80 carbon atoms. In some embodiments, the PEG comprises a chain having about 20-2000 carbon atoms.

In some embodiments, the PEG is about 2000 Da.

In some embodiments, the siRNA of the invention may be delivered via a nanoparticle. In some embodiments, the nanoparticle may be a lipid nanoparticle. In some embodiments, the nanoparticle may comprise a calcium phosphate nanoparticle. In some embodiments, the calcium phosphate nanoparticle comprises more than one siRNA.

In some embodiments, the nanoparticle is non-toxic, stable, and degradable in vivo releasing the siRNA. In some embodiments, the nanoparticle comprises a calcium phosphate complex. The calcium phosphate complex is stable and non-toxic.

In some embodiments, the nanoparticles, as exemplified in FIG. 1, are termed “nanoparticle compositions.”

In some embodiments, the nanoparticle, (e.g., the lipid nanoparticle, the calcium phosphate nanoparticle, etc.) comprises particles that are between 80-250 nm in diameter.

Calcium phosphate nanoparticles are stable and non-toxic. Upon delivery of the siRNA encapsulated in the calcium phosphate nanoparticle, the nanoparticles disperse readily, and upon dissociation and release of the siRNA, the residual nanoparticle materials, calcium and phosphate are naturally occurring non-toxic materials, suitable for human in vivo use. In some embodiments, the siRNA described herein comprises delivery via calcium phosphate nanoparticles. In some embodiments, the calcium phosphate nanoparticle is prepared with or without poly-ethylene glycol (PEG). PEGs may act as the terminal surface group of the nanoparticle. In some embodiments, PEG acts as a steric protectant from aggregation, endows a longer half-life in serum, enhances serum dispersibility, along with tumor permeability and availability of the siRNA.

In some embodiments, the siRNA of the invention may be associated with a cell targeting moiety. In some embodiments, the cell targeting moiety is added into the formulation mixture during calcium phosphate nanoparticle formulation.

In an exemplary method for siRNA-calcium phosphate nanoparticle formulation, the first stage of synthesis involves the conjugation of the siRNA to PEG moieties. The phosphoamide chemistry involves conjugation of siRNA strand having 5′-Phosphate (5′-P) end groups to an amine-terminated methoxy-PEG molecule, while the thioester chemistry involves conjugating siRNA with 5′sulfhydril end groups to a maleimide terminated methoxy PEG molecule. To achieve the phosphoamide conjugation, 0.12M N-(3-Dimethylaminopropyl)-N′-ethylcarbodiimide hydrochloride, 47.6 uM single stranded siRNA (inactive strand containing 5′-P groups) and 0.238M amine-PEG are dissolved in 0.1M imidazole at pH 6 and incubated 18 hrs at 50° C. This reaction typically yields 15-30% conjugation. Alternatively, the addition of 0.1M MES buffer, pH 4, into the phosphoamide reaction mixture results in conjugation efficiency of 75-90%. For the thioether chemistry, 200 uM single stranded siRNA (inactive strand containing 5′ P groups) is combined with 0.476M amine-PEG in the presence of 0.02M DTT in a 0.1M Tris HCl buffer and the reaction is incubated at 25° C. 18 hrs. This reaction yields approximately 50% conjugation. These reactions have been performed using 2 kDa and 5 kDa PEG molecules. Following conjugation, the inactive (sense) strand is annealed to the unconjugated active (antisense) strand RNA in the reaction mix by heating to 70° C. followed by slow cooling. For the highly efficient (75-90%) phosphoamide reaction, unconjugated, annealed siRNA containing either 5′P groups on both the active (antisense) and inactive (sense) strand or one 5′ P group on the active (antisense) strand are added to the reaction mixture to facilitate desalt purification, as described below. The presence of 5′ P groups on the inactive (sense) strand results in a more negatively charged particle while the absence of these groups shifts the particle charge closer to neutral.

Purification can be achieved using a modified desalting procedure that is carried out by combining 1 volume of annealed conjugation reaction mixture, 1 volume of 5M sodium acetate and 13 parts 70:30 ethyl acetate:methanol. Following incubation at −80° C. for at least 18 hours, the mixture is centrifuge for 90 minutes at 21,000 g and 4° C., the supernatant is decanted. A second desalt with methanol follows the above procedure and pellets are washed with 80% methanol and then dried. This procedure facilitates the separation of both the free and conjugated siRNA from excess PEG and reaction components including imidazole, carbodiimide, DTT, Tris and most salts.

Conjugated siRNA thus prepared is added to Na₂HPO₄ in aqueous solution. The solution is then mixed with CaCl₂) that has been adjusted to pH 8.5-10.5, most optimally pH 9. A final molar ratio of calcium to phosphate between 0.9-1.5:1, most optimally 1.2:1 is required to form stable particles. Optimal component concentrations are 5 mM Na₂HPO₄, 4.05 mM CaCl₂ and 80 uM-225 uM siRNA. However, the siRNA concentration may be varied depending on the sequence and length of sequence. Shorter, 21-bp siRNA duplexes, typically require higher concentrations of approximately 175-225 uM siRNA to stabilize a monomodal particle size distribution, while longer siRNA duplexes, 25-bp, require less material, typically approximately 80-125 uM siRNA to yield the same particle size. Some variation in particle size distribution about the lower and higher siRNA concentrations is seen between sequences of the same length. Higher siRNA concentrations are required to attain monomodal particle size distributions of <250 nm if the inactive (sense) strand does not contain 5′—P groups.

Particles are washed via centrifuge filtration with 5% dextrose containing calcium and phosphate to remove unincorporated siRNA and siRNA-PEG conjugates. Alternatively, unincorporated siRNA, Ca, Cl, Na and PO₄ can be removed from the suspension by ultracentrifugation at 132,000 g, which results in particle collection in the bottom 10% of the sample volume. Separation of the bottom 10% of the sample volume containing the particles reduces residual unincorporated components by up to 90%. Capture of the siRNA within the particle is sequence dependent and ranges from 10-25% of the siRNA added in the synthesis. The synthesis describe herein results in calcium phosphate nanoparticles containing siRNA with monomodal particle size distributions of ≤160 nm. By incubating the particles in 1-10 mM CaCl₂ at 37° C. for 1-24 h the surface charge of the nanoparticle may be altered.

The size of siRNA calcium phosphate nanoparticles, within a specified concentration range and molar ratio of calcium and phosphate, is controlled via the concentrations of siRNA-PEG added in the synthesis.

In one embodiment siRNAs may be designed wherein a 5′- or a 3′ overhang region may be chemically crosslinked to a targeting moiety. In some embodiments, the siRNA is attached via a linker to a targeting moiety. In some embodiments, the linker is a non-reactive short linker, e.g., a short peptide linker. In some embodiments, the linker may be a bioactive peptide linker. In some embodiment, the linker is a short PEG molecule, comprising 10-12 carbon atoms in a chain. In some embodiments, the PEG is PEG 2000. In some embodiments, the PEG molecule is crosslinked to the 5′ end or the 3′-end of one strand of the siRNA. In some embodiments, the crosslinker is a maleimide functional bi-conjugational linker.

In some embodiments, the crosslinker is a short polymer. In some embodiments, the polymer is a functional polymer, a conjugated polymer or a copolymer, for example, a PLGA-PEG, a PLA-PEG, a PCA-PEG, a lipid PEG, a polylysine PEG.

In some embodiments, the siRNA delivery system is a Solid Nucleic Acid Lipid Nanoparticle (SNALP) technology, which utilizes cationic or charge-conversional lipids with polyethylene glycol (PEG) surface groups.

In some embodiments, the siRNA delivery system is a cyclodextrin-based delivery system.

In some embodiments, the siRNA is conjugated directly or via a linker to an aptamer. Aptamers are synthetically tractable ligands for both diagnostic and therapeutic purposes, and are often referred to as nucleic acid ligands, “oligobodies,” or “chemical antibodies.” Aptamers that bind cell surface receptors are readily endocytosed. Aptamers are characterized by an ability to fold into complex tertiary structures and to bind with high affinity (low nM to high pM equilibrium dissociation constants) and specificity to their targets. Isolation of aptamers specific for a target of interest involves iterative rounds of a process termed SELEX (systematic evolution of ligands by exponential enrichment; (Tuerk and Gold, 1990, Proc. Natl. Acad. Sci. USA. 89, 6988-6992). For the SELEX process, an aptamer library is incubated with a protein target. The protein-bound aptamers are then specifically recovered. These sequences are amplified with PCR or RT-PCR. Single-stranded RNA or DNA sequences representing the recovered sequences are then generated from these PCR products, and used in the subsequent selection round. Exemplary aptamers known to one of skill in the art may include tenascin-C aptamer (TTA1), PMSA aptamer, CTLA-4 aptamer, HIV-bivalent aptamer etc., depending on the cancer type, target etc. (Thiel et al., Oligonucleotides. 2009 Sep. 1; 19(3): 209-222).

### Selective Targeting of Cancer Cells

In one aspect of the invention, provided herein is a method for inhibiting a shorter isoform or variant inside a diseased cell, e.g., a cancer cell, comprising, administering an inhibitor of the shorter isoform in a pharmaceutical formulation that further comprises a targeting moiety that brings the inhibitor in contact with the diseased cell. In some embodiments, the targeting moiety is a cancer cell-targeting moiety. In some embodiments, the formulation comprises a nanoparticle, wherein the nanoparticle comprises the inhibitor and the targeting moiety, and wherein the nanoparticle displays the targeting moiety on the surface of the nanoparticle. The targeting moiety can bind to a target protein on a target cell (e.g. cancer cell) surface, and deliver the therapeutically active siRNA compound into the cancer cell. In some embodiments, the targeting moiety is a ligand, an antibody, an scFv, a single domain antibody, a single chain antibody, a diabody—that binds to a protein on a cancer cell surface. In some embodiments, the targeting moiety could be a nucleic acid molecule. In some embodiments, the targeting moiety is an aptamer.

Aptamers that bind cell surface receptors are readily endocytosed. Aptamers are characterized by an ability to fold into complex tertiary structures of biomolecules and to bind with high affinity (low nM to high pM equilibrium dissociation constants) and specificity to their targets. Isolation of aptamers specific for a target of interest involves iterative rounds of a process termed SELEX (systematic evolution of ligands by exponential enrichment; (Tuerk and Gold, 1990, Proc. Natl. Acad. Sci. USA. 89, 6988-6992). For the SELEX process, an aptamer library is incubated with a protein target. The protein-bound aptamers are then specifically recovered. These sequences are amplified with PCR or RT-PCR. Single-stranded RNA or DNA sequences representing the recovered sequences are then generated from these PCR products, and used in the subsequent selection round. Exemplary aptamers known to one of skill in the art may include tenascin-C aptamer (TTA1), PMSA aptamer, CTLA-4 aptamer, HIV-bivalent aptamer etc., depending on the cancer type, target etc. (Thiel et al., Oligonucleotides. 2009 Sep. 1; 19(3): 209-222). As contemplated herein, any one of the disclosed aptamers could be suitable for the purpose of the invention, as long as the aptamer can be conjugated to or assembled into a pharmaceutical composition or formulation carrying the therapeutically active compound, e.g., the exemplary anti-RIZ2 siRNA.

### Anti-Nucleolin Aptamer

An aptamer is a nucleic acid polymer that can bind to another biomolecule specifically by means of its 3-dimensional structure with or without the complete dependence on its sequence of nucleobases. In one aspect, the method described herein comprises targeting a pharmaceutical compound, e.g., an anti-PRDM2 (RIZ2) agent, such as a small molecule or an inhibitory RNA to a cancer cell, by associating the pharmaceutical compound to a targeting moiety (e.g., aptamer) that targets a cancer cell. In some embodiments, the targeting moiety is capable of binding to a cell surface protein or glycoprotein that is expressed on a cancer cell surface. Preferably, the cell surface protein or glycoprotein that is expressed on a cancer cell surface is predominantly expressed on the surface of a cancer cell, and is not generally present on the surface of a healthy cell e.g., a non-cancer cell, thereby rendering precision and specificity to the targeting moiety to deliver a therapeutic compound to a cancer cell without generally affecting the non-cancer cell, where the target cell surface protein is not expressed. In some embodiments, the targeting moiety is capable of binding to a cancer cell-specific cell surface protein or glycoprotein. In some embodiments, a cancer cell-specific surface protein can be a protein that overexpresses in a cancer cell, whereas its expression is considerably lower in a non-cancer cell, such that the targeting moiety binds to a cancer cell preferably over a non-cancer cell, due to the sheer abundance of the protein in cancer cell. A cancer cell-specific protein can be any cell surface biomolecule, a protein a peptide, a conjugated protein, a lipoprotein, a glycoprotein, a carbohydrate molecule, a lipid molecule, that is displayed on the cell surface. In some embodiments, the cancer cell-specific cell surface protein is nucleolin, that is known to be preferentially transported to the cell surface of cancer cells, to target the pharmaceutical compound to the site of the tumor with a ligand that recognizes the nucleolin on the cancer cell surface.

In some embodiments, the aptamer is AS1411. AS1411 is a DNA aptamer that can bind to nucleolin. Use of such an aptamer could be highly beneficial in targeting the siRNA to a cancer cell, and enhancing the efficacy of the anti-PRDM siRNA drug action. In some embodiments, use of the AS1411 aptamer enhances the specificity of the anti-PRDM siRNA drug. AS1411 is a synthetic 26-28 nucleotide phosphodiester oligodeoxynucleotide (i.e. unmodified DNA). In some embodiments, AS1411 has a DNA sequence set forth as: 5′-TTGGTGGTGGTGGTTGTGGTGGTGGTGG-3′(SEQ ID NO: 21). In some embodiments, the cancer cell targeting moiety is a DNA aptamer having a sequence that is 26-28 nucleotides long and comprises a sequence that is at least 90% identical to SEQ ID NO: 21. In some embodiments, the cancer cell targeting moiety comprises a sequence that is at least 91% identical to SEQ ID NO: 21. In some embodiments, the cancer cell targeting moiety comprises a sequence that is at least 92% identical to SEQ ID NO: 21. In some embodiments, the cancer cell targeting moiety comprises a sequence that is at least 93% identical to SEQ ID NO: 21. In some embodiments, the cancer cell targeting moiety comprises a sequence that is at least 94% identical to SEQ ID NO: 21. In some embodiments, the cancer cell targeting moiety comprises a sequence that is at least 95% identical to SEQ ID NO: 21. In some embodiments, the cancer cell targeting moiety comprises a sequence that is at least 96% identical to SEQ ID NO: 21. In some embodiments, the cancer cell targeting moiety comprises a sequence that is at least 97% identical to SEQ ID NO: 21. In some embodiments, the cancer cell targeting moiety comprises a sequence that is at least 98% identical to SEQ ID NO: 21. In some embodiments, the cancer cell targeting moiety comprises a sequence that is at least 99% identical to SEQ ID NO: 21. In some embodiments, the cancer cell targeting moiety comprises a sequence of SEQ ID NO: 21. In some embodiments, the cancer cell targeting moiety comprises a sequence having less than 3 nucleotides, less than 2 nucleotides, or 1 nucleotide different from that of SEQ ID NO: 21. In some embodiments, the aptamer is a derivative of AS1411. In some embodiments, the derivative AS1411 comprises one or more chemical modifications (e.g., LNA-AS1411 and U-AS1411).

In some embodiments, the cancer cell targeting moiety comprises pegylated aptamers. In some embodiments, the cancer cell targeting moiety comprises pegylated AS1411. In some embodiments, the AS1411 is pegylated with a PEG as described here. In some embodiments, the PEG comprises a chain having about 20-about 120 carbon atoms. In some embodiments, the PEG comprises a chain having about 40-120 carbon atoms. In some embodiments, the PEG comprises a chain having about 60-120 carbon atoms. In some embodiments, the PEG comprises a chain having about 80-120 carbon atoms. In some embodiments, the PEG comprises a chain having about 100-120 carbon atoms. In some embodiments, the PEG comprises a chain having about 20-40 carbon atoms. In some embodiments, the PEG comprises a chain having about 20-60 carbon atoms. In some embodiments, the PEG comprises a chain having about 20-80 carbon atoms. In some embodiments, the PEG comprises a chain having about 20-100 carbon atoms. In some embodiments, the PEG comprises a chain having about 20-80 carbon atoms. In some embodiments, the PEG comprises a chain having about 20-1000 carbon atoms. In some embodiments, the PEG comprises a chain having about 20-80 carbon atoms. In some embodiments, the PEG comprises a chain having about 20-2000 carbon atoms. In some embodiments, the polyethylene glycol (PEG) chain has a molecular weight of 2000. In some embodiments, the PEG has a molecular weight of about 400-5000 Da. In some embodiments, the PEG has a molecular weight of about 1000-3000 Da. In some embodiments, the PEG has a molecular weight of about 400-10,000 Da.

In some embodiments, AS1411 has a DNA sequence set forth as 5′-GGTGGTGGTGGTTGTGGTGGTGGTGG-3′ (SEQ ID NO: 22). It is well known that AS1411 can form G-quadruplex-containing structures. In some embodiments, the AS1411 is a monomer. In some embodiments, the AS1411 is a multiplex structure.

AS1411 is a DNA aptamer cancer cell targeting moiety used in a formulation described herein for delivery of a PRDM inhibitor that targets a specific PRDM isoform; AS1411 can bind to a nuclear protein, nucleolin, a phosphoprotein that is abundantly expressed in cancer cells. In cancer cells, nucleolin is exposed on the cell surface. In cancer cell, nucleolin accumulates on the cancer cell surface. Nucleolin is aberrantly expressed on a cancer cell surface. This aberrant expression is not readily observed in non-cancer cells. Nucleolin has been understood to be predominantly nuclear (nucleolar) protein in normal (e.g., non-cancer) cells. Known to be a protein with predominantly nucleolar localization, nucleolin plays a central role in polymerase 1 transcription, ribosomal RNA synthesis and ribosome biogenesis. However, presence of nucleolin is recently confirmed to be extranucleolar in many different circumstances, for example, in the nucleoplasm, cytoplasm and on the cell membrane. In cancer cells, nucleolin overexpression influences cell survival, proliferation and invasion through its action on different cellular pathways (Berger, C., et al., 2015, BioChimie, Vol. 113, p 78-85). Nucleolin was found to be overexpressed in colorectal cancer, gastric cancer, breast cancer, glioblastoma, leukemia, cervical cancer, with extra-nucleolar, e.g., cytoplasmic and cell surface expression. Nucleolin can be transported to the cell surface by an unconventional secretory pathway in response to mitogenic stimuli. The functioning of AS1411 is related to selective binding to non-nuclear nucleolin accumulated on the surface of cancer cells. Nucleolin can bind to a variety of quadruplex structures, such as those exhibited by AS1411. In some embodiments, AS1411 may exhibit some antiproliferative activity. In some embodiments, the antiproliferative activity of AS1411 is lower compared to the AS1411 in combination or association with the pharmaceutical compound described herein, the pharmaceutical compound is the compound that is the therapeutically active component of the composition, for example, the anti-RIZ2 (PRDM2) specific siRNA. In some embodiments, the antiproliferative activity of AS1411 is at least 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.75, 2.8%, 2.9%, 3%, 4%, 5%, 6%, 7%, 8%, 9%, 10%, 15%, 20%, 25%, 30%, 35%, 40%, or 50% lower than the formulation comprising siRNA described herein in combination with AS1411, as demonstrated in an in vitro cancer cell proliferation assay. In some embodiments, the anti-proliferative activity of the pharmaceutical composition comprising the siRNA disclosed herein, encapsulated in the nanoparticle delivery vehicle and together with the targeting moiety, e.g., AS1411, is at least 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.75, 2.8%, 2.9%, 3%, 4%, 5%, 6%, 7% 8%, 9% 10%, 15%, 20%, 25%, 30%, 35%, 40%, or 50% higher than the anti-proliferative activity of AS1411 alone. In some embodiments, AS1411 shows tumor regression in an experimental mouse tumor model, wherein the tumor regression is lower than that exhibited by a formulation comprising both the siRNA described herein and AS1411 by at least 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.75, 2.8%, 2.9%, 3%, 4%, 5%, 6%, 7%, 8%, 9%, 10%, 15%, 20%, 25%, 30%, 35%, 40%, or 50% as seen by tumor mass measurements over a given time. In some embodiments, the tumor regression function of the pharmaceutical composition comprising the siRNA disclosed herein, encapsulated in the nanoparticle delivery vehicle and together with the targeting moiety, e.g., AS1411, is at least 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.75, 2.8%, 2.9%, 3%, 4%, 5%, 6%, 7%, 8%, 9%, 10%, 15%, 20%, 25%, 30%, 35%, 40%, or 50% higher than the anti-proliferative activity of AS1411 alone. In some embodiments, the siRNA described herein, together with the AS1411 can exhibit a tumor static effect on tumor progression when applied in vivo, when tested in a suitable animal model. In some embodiments, the siRNA described herein, together with the AS1411 can exhibit a tumoricidal effect when applied in vivo, when tested in a suitable animal model. A suitable animal model could be a mouse xenograft model described herein. Briefly, a mouse xenograft model is a disease model in which an immunodeficient or immunocompromised mouse is injected with human tumor cells, which then develop the human tumor. Drug actions are routinely tested in such mouse models developed on site or can be commercially available. Effect of the drug on the tumor thus formed can be tested on the animal model by monitoring the tumor size (growth), and by molecular and biochemical analysis of the tumor or tissue harvested from the animal.

In some embodiments, the use of AS1411 as a targeting moiety described in the instant disclosure stems from the recognition that targeting nucleolin could target early transforming and metastatic cells. During the progression of cancer, cancer cells will break away from cancer tissue, and intrude into and drift in the circulation before implanting in novel regions. It is well known that cancer cells can undergo epithelial mesenchymal transition (EMT) to enhance their metastatic potential. Some studies implied that the disturbance of nucleolin could inhibit the process of EMT. For instance, transfecting cells with nucleolin-targeted small interfering RNA could result in the inhibition of the EMT phenotypes. In some embodiments, an anti-nucleolin targeting moiety can target a cancer cell that is undergoing EMT. In some embodiments, an anti-nucleolin targeting moiety can target a cancer cell that is undergoing a break away from cancer tissue, thereby metastasizing elsewhere. In some embodiments, the anti-nucleolin targeting moiety is sufficiently short and can be conjugated to a drug, or a nucleic acid molecule, such as a therapeutic nucleic acid molecule, e.g., an siRNA. In some embodiments, the anti-nucleolin targeting moiety is AS 1411.

In some embodiments, the targeting moiety is directly conjugated to the pharmaceutical compound, e.g., siRNA in a composition. In some embodiments, the targeting moiety is conjugated to a component. In some exemplary method of preparing siRNA nanoparticles with a targeting moiety, wherein the targeting moiety, e.g. the AS 1411 is conjugated to PEG first; the AS1411-PEG and the siRNA-PEG molecules prepared as described earlier are introduced into the nanoparticle assembly reaction.

### Pharmaceutical Compositions

Provided herein is a pharmaceutical composition comprising a RIZ2 inhibitor and a pharmaceutically acceptable ingredient.

In some embodiments, the RIZ2 inhibitor is a short peptide sequence that inhibits the action of a RIZ2 protein. In some embodiments, the RIZ 2 inhibitor is a small molecule.

In some embodiments, the RIZ2 inhibitor comprises one or more polynucleotide molecules and a delivery system.

In some embodiments, the RIZ2 inhibitor comprises an antisense oligonucleotide (ASO).

In some embodiments, the RIZ2 inhibitor comprises an inhibitory RNA molecule, such as a double stranded siRNA molecule. In some embodiments, the inhibitory RNA molecule hybridizes to at least 10 contiguous nucleobases on the PRDM2 gene or a PRDM2 gene product. In some embodiments, the inhibitory RNA molecule is about 10 to about 21 nucleotides in length. In some embodiments, the RIZ2 inhibitor comprises more than one siRNA.

Provided herein is an siRNA formulated in a pharmaceutical composition for administering by subcutaneous injection.

Provided herein is an siRNA formulated in a pharmaceutical composition for administering by intravenous injection.

Provided herein is an siRNA formulated in a pharmaceutical composition for systemic administration.

Provided herein is an siRNA formulated in a pharmaceutical composition for administering locally or topically.

In some embodiments, the pharmaceutically acceptable excipient may be pathogen free aqueous component, e.g., pathogen free water. In some embodiments, the pharmaceutically acceptable excipient may be a suitable buffer at neutral pH, e.g., phosphate buffered saline. In some embodiments, the pharmaceutically acceptable excipient may be a slightly acidic aqueous solution. In some embodiments, the pharmaceutically acceptable excipient may comprise glycol, glycerol, DMSO, soluble components, sugars, salts etc. In some embodiments, the formulation comprises a bulking agent, e.g. sucrose, trehalose, mannitol, glycine, lactose and/or raffinose, to impart a desired consistency to the formulation and/or stabilization of formulation components. In some embodiments, excipient commonly used for topical administration may be used, as is known to one of skill in the art.

Provided herein is a pharmaceutical composition comprising an inhibitory double-stranded RNA comprising a sequence of SEQ ID NO: 1, or a sequence having at least 90% identity to SEQ ID NO: 1. In some embodiments, provided herein is a pharmaceutical composition comprising an inhibitory double-stranded RNA comprising a sequence of SEQ ID NO: 10, or a sequence having at least 90% identity to SEQ ID NO: 10. Provided herein is a pharmaceutical composition comprising an inhibitory double-stranded RNA comprising the sequence of SEQ ID NO: 1 as sense strand and the sequence of SEQ ID NO: 10 as antisense strand.

Provided herein is a pharmaceutical composition comprising an inhibitory double-stranded RNA comprising a sequence of SEQ ID NO: 6, or a sequence having at least 90% identity to SEQ ID NO: 6. In some embodiments, provided herein is a pharmaceutical composition comprising an inhibitory double-stranded RNA comprising a sequence of SEQ ID NO: 11, or a sequence having at least 90% identity to SEQ ID NO: 11. Provided herein is a pharmaceutical composition comprising an inhibitory double-stranded RNA comprising the sequence of SEQ ID NO: 6 as sense strand and the sequence of SEQ ID NO: 11 as antisense strand.

Provided herein is a pharmaceutical composition comprising an inhibitory double-stranded RNA In some embodiments, provided herein is a RIZ2 inhibitor comprising a double stranded polynucleotide comprising a sense strand of SEQ ID NO: 2 or a sequence having at least 90% identity to SEQ ID NO: 2, and an antisense strand of SEQ ID NO: 12, or a sequence having at least 90% identity to SEQ ID NO:12.

In some embodiments, provided herein is a pharmaceutical composition comprising a RIZ2 inhibitor comprising a double stranded polynucleotide comprising a sense strand of SEQ ID NO: 3, or a sequence having at least 90% identity to SEQ ID NO:3, and an antisense strand of SEQ ID NO: 13, or a sequence having at least 90% identity to SEQ ID NO:13.

In some embodiments, provided herein is a pharmaceutical composition comprising a RIZ2 inhibitor comprising a double stranded polynucleotide comprising a sense strand of SEQ ID NO: 4, or a sequence having at least 90% identity to SEQ ID NO:4, and an antisense strand of SEQ ID NO: 14, or a sequence having at least 90% identity to SEQ ID NO:14.

In some embodiments, provided herein is a pharmaceutical composition comprising a RIZ2 inhibitor comprising a double stranded polynucleotide comprising a sense strand of SEQ ID NO: 5, or a sequence having at least 90% identity to SEQ ID NO:5, and an antisense strand of SEQ ID NO: 15, or a sequence having at least 90% identity to SEQ ID NO:15.

In some embodiments, provided herein is a pharmaceutical composition comprising a RIZ2 inhibitor comprising a double stranded polynucleotide comprising a sense strand of SEQ ID NO: 7, or a sequence having at least 90% identity to SEQ ID NO:7, and an antisense strand of SEQ ID NO: 16, or a sequence having at least 90% identity to SEQ ID NO: 16.

In some embodiments, provided herein is a pharmaceutical composition comprising a RIZ2 inhibitor comprising a double stranded polynucleotide comprising a sense strand of SEQ ID NO: 8, or a sequence having at least 90% identity to SEQ ID NO:8, and an antisense strand of SEQ ID NO: 17 or a sequence having at least 90% identity to SEQ ID NO:17.

In some embodiments, provided herein is a pharmaceutical composition comprising a RIZ2 inhibitor comprising a double stranded polynucleotide comprising a sense strand of SEQ ID NO: 9, or a sequence having at least 90% identity to SEQ ID NO:9, and an antisense strand of SEQ ID NO: 18, or a sequence having at least 90% identity to SEQ ID NO:18.

In some embodiments, the pharmaceutical composition comprises one or more siRNAs described above, and a siRNA delivery system, such as a liposome, a nanoparticle, as disclosed herein. In some embodiments, the pharmaceutical composition having the delivery system comprises a cell targeting moiety, wherein the cell targeting moiety is a ligand, and antibody or an antibody fragment, a single chain antibody, a peptide or an aptamer. In some embodiments, the cell targeting moiety is a cell-penetrating peptide. In some embodiments, the delivery system comprises one or more of lipids, cyclodextrin, chitosan, carbohydrate polymers, elastin-like polymers (ELP), calcium phosphate polymers or combinations thereof.

Provided herein is a pharmaceutical composition comprising the RIZ2 inhibitor comprises comprising: (i) an siRNA comprising a double stranded RNA of 10-21 nucleotides that have homology to human PRDM2/RIZ2 gene; covalently linked to a (ii) a PEG molecule, linked to a (iii) cell targeting moiety; wherein the cell targeting moiety is an aptamer.

In some embodiments, the pharmaceutical composition comprises one or more chemotherapeutic drugs. Exemplary chemotherapeutic drugs may include but are not limited to: obinutuzumab, bendamustine, chlorambucil, cyclophosphamide, ibrutinib, methotrexate, cytarabine, dexamethasone, cisplatin, bortezomib, fludarabine, idelalisib, acalabrutinib, lenalidomide, venetoclax, cyclophosphamide, ifosfamide, etoposide, pentostatin, melphalan, carfilzomib, ixazomib, panobinostat, daratumumab, elotuzumab, thalidomide, lenalidomide, or pomalidomide, or a combination thereof.

In some embodiments, the therapeutic composition may comprise additional drugs, for example, checkpoint inhibitors, e.g., a PD1 inhibitor, a PD-L1 inhibitor, or a CTLA4 inhibitor or a combination thereof.

In some embodiments, the pharmaceutical composition comprises an siRNA targeting a PRDM2 transcript, e.g., RIZ2 transcript, (for example, ARIZ-047 siRNA) and further comprises an inhibitor of a WNT pathway signaling molecule. In some embodiments, the inhibitor may be a small molecule, a nucleic acid molecule, a peptide molecule, an antibody or a fragment thereof, or a conjugated molecule.

In some embodiments, the inhibitor may be present in an effective amount within the composition to effectively downregulate the action of the WNT signaling member or molecule.

In some embodiments, the pharmaceutical composition comprises an the pharmaceutical composition comprises an siRNA targeting a PRDM2 transcript, e.g., RIZ2 transcript, (for example, ARIZ-047 siRNA) and further comprises an activator of the EGR1 pathway. In some embodiments, the activator may be a small molecule, a nucleic acid molecule such as an siRNA, and antisense oligonucleotide, an miRNA, a peptide molecule, an antibody or a fragment thereof, or a conjugated molecule.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present disclosure is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a siRNA therapeutic described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that can be administered in combination with the siRNA therapeutic described herein include coadministration with chemotherapeutic agents. Thus, in some embodiments, the method or treatment involves the administration of an agent described herein in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, PA.

Useful classes of chemotherapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in the present disclosure include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2′″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6 mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In some embodiments, the additional therapeutic agent is albumin-bound paclitaxel.

In some embodiments, an additional therapeutic agent comprises an agent such as a small molecule. For example, treatment can involve the combined administration of an agent of the present disclosure with a small molecule that acts as an inhibitor against tumor-associated antigens including, but not limited to, EGFR, HER2 (ErbB2), and/or VEGF. In some embodiments, an agent of the present disclosure is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor. In another embodiment, the additional therapeutic agent is chemotherapy or other inhibitors that reduce the number of Treg cells. In certain embodiments, the therapeutic agent is cyclophosphamide or an anti-CTLA4 antibody. In another embodiment, the additional therapeutic reduces the presence of myeloid-derived suppressor cells. In a further embodiment, the additional therapeutic is carbotaxol. In another embodiment, the additional therapeutic agent shifts cells to a T helper 1 response. In a further embodiment, the additional therapeutic agent is ibrutinib.

In some embodiments, an additional therapeutic agent comprises a biological molecule, such as an antibody. For example, treatment can involve the combined administration of an agent of the present disclosure with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

The agents and compositions provided herein may be used alone or in combination with conventional therapeutic regimens such as surgery, irradiation, chemotherapy and/or bone marrow transplantation (autologous, syngeneic, allogeneic or unrelated). A set of tumor antigens can be useful, e.g., in a large fraction of cancer patients.

In some embodiments, at least one or more chemotherapeutic agents may be administered in addition to the composition comprising an immunogenic vaccine. In some embodiments, the one or more chemotherapeutic agents may belong to different classes of chemotherapeutic agents.

Examples of chemotherapy agents include, but are not limited to, alkylating agents such as nitrogen mustards (e.g. mechlorethamine (nitrogen mustard), chlorambucil, cyclophosphamide (Cytoxan®), ifosfamide, and melphalan); nitrosoureas (e.g. N-Nitroso-N-methylurea, streptozocin, carmustine (BCNU), lomustine, and semustine); alkyl sulfonates (e.g. busulfan); tetrazines (e.g. dacarbazine (DTIC), mitozolomide and temozolomide (Temodar®)); aziridines (e.g. thiotepa, mytomycin and diaziquone); and platinum drugs (e.g. cisplatin, carboplatin, and oxaliplatin); non-classical alkylating agents such as procarbazine and altretamine (hexamethylmelamine); anti-metabolite agents such as 5-fluorouracil (5-FU), 6-mercaptopurine (6-MP), capecitabine (Xeloda®), cladribine, clofarabine, cytarabine (Ara-C®), decitabine, floxuridine, fludarabine, nelarabine, gemcitabine (Gemzar®), hydroxyurea, methotrexate, pemetrexed (Alimta®), pentostatin, thioguanine, Vidaza; anti-microtubule agents such as vinca alkaloids (e.g. vincristine, vinblastine, vinorelbine, vindesine and vinflunine); taxanes (e.g. paclitaxel (Taxol®), docetaxel (Taxotere®)); podophyllotoxin (e.g. etoposide and teniposide); epothilones (e.g. ixabepilone (Ixempra®)); estramustine (Emcyt®); anti-tumor antibiotics such as anthracyclines (e.g. daunorubicin, doxorubicin (Adriamycin®, epirubicin, idarubicin); actinomycin-D; and bleomycin; topoisomerase I inhibitors such as topotecan and irinotecan (CPT-11); topoisomerase II inhibitors such as etoposide (VP-16), teniposide, mitoxantrone, novobiocin, merbarone and aclarubicin; corticosteroids such as prednisone, methylprednisolone (Solumedrol®), and dexamethasone (Decadron®); L-asparaginase; bortezomib (Velcade®); immunotherapeutic agents such as rituximab (Rituxan®), alemtuzumab (Campath®), thalidomide, lenalidomide (Revlimid®), BCG, interleukin-2, interferon-alfa and cancer vaccines such as Provenge®; hormone therapeutic agents such as fulvestrant (Faslodex®), tamoxifen, toremifene (Fareston®), anastrozole (Arimidex®), exemestan (Aromasin®), letrozole (Femara®), megestrol acetate (Megace®), estrogens, bicalutamide (Casodex®), flutamide (Eulexin®), nilutamide (Nilandron®), leuprolide (Lupron®) and goserelin (Zoladex®); differentiating agents such as retinoids, tretinoin (ATRA or Atralin®), bexarotene (Targretin®) and arsenic trioxide (Arsenox®); and targeted therapeutic agents such as imatinib (Gleevec®), gefitinib (Iressa®) and sunitinib (Sutent®). In some embodiments, the chemotherapy is a cocktail therapy. Examples of a cocktail therapy includes, but is not limited to, CHOP/R-CHOP (rituxan, cyclophosphamide, hydroxydoxorubicin, vincristine, and prednisone), EPOCH (etoposide, prednisone, vincristine, cyclophosphamide, hydroxydoxorubicin), Hyper-CVAD (cyclophosphamide, vincristine, hydroxydoxorubicin, dexamethasone), FOLFOX (fluorouracil (5-FU), leucovorin, oxaliplatin), ICE (ifosfamide, carboplatin, etoposide), DHAP (high-dose cytarabine [ara-C], dexamethasone, cisplatin), ESHAP (etoposide, methylprednisolone, cytarabine [ara-C], cisplatin) and CMF (cyclophosphamide, methotrexate, fluouracil).

In certain embodiments, an additional therapeutic agent comprises a second immunotherapeutic agent. In some embodiments, the additional immunotherapeutic agent includes, but is not limited to, a colony stimulating factor, an interleukin, an antibody that blocks immunosuppressive functions (e.g., an anti-CTLA-4 antibody, anti-CD28 antibody, anti-CD3 antibody, anti-PD-1 antibody, anti-PD-L1 antibody, anti-TIGIT antibody), an antibody that enhances immune cell functions (e.g., an anti-GITR antibody, an anti-OX-40 antibody, an anti-CD40 antibody, or an anti-4-1BB antibody), a toll-like receptor (e.g., TLR4, TLR7, TLR9), a soluble ligand (e.g., GITRL, GITRL-Fc, OX-40L, OX-40L-Fc, CD40L, CD40L-Fc, 4-1BB ligand, or 4-1BB ligand-Fc), or a member of the B7 family (e.g., CD80, CD86). In some embodiments, the additional immunotherapeutic agent targets CTLA-4, CD28, CD3, PD-1, PD-L1, TIGIT, GITR, OX-40, CD-40, or 4-1BB.

In some embodiments, the additional therapeutic agent is an immune checkpoint inhibitor. In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAG3 antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-PD-1 antibody selected from the group consisting of: nivolumab (OPDIVO), pembrolizumab (KEYTRUDA), pidilzumab, MEDI0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MEDI4736), and avelumab (MSB0010718C). In some embodiments, the additional therapeutic agent is an anti-CTLA-4 antibody selected from the group consisting of: ipilimumab (YERVOY) and tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566.

In some embodiments, the siRNA therapeutic can be administered in combination with a biologic molecule selected from the group consisting of: adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), FLT-3L, macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF-β, TNF-α, VEGF, PIGF, gamma-IFN, IL-1, IL-2, IL-3, IL-4, IL-5, IL-6, IL-7, IL-12, IL-15, and IL-18.

In some embodiments, treatment with an siRNA therapeutic described herein can be accompanied by surgical removal of tumors, removal of cancer cells, or any other surgical therapy deemed necessary by a treating physician.

In certain embodiments, treatment involves the administration of an siRNA therapeutic described herein in combination with radiation therapy. Treatment with an agent can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously.

In some embodiments, the activator may be present in an effective amount in the composition to effectively activate or upregulate the action of the member or molecule of the EGR1 pathway.

### Therapeutic and Diagnostic Methods

Therapeutic compositions described above comprising one or more inhibitory RNA molecules targeting RIZ2 may be used to treat cell proliferative disorders in a subject. In some embodiments, the subject is a human. In some embodiments, the cell proliferative disorder is a cancer. In some embodiments, the cell proliferative disorder is associated with or a result of cell cycle deregulation, loss of cell cycle checkpoint inhibition, and/or RIZ1/RIZ2 imbalance. It has been well known that histone H3 K9 methyltransferase activity of RIZ1 plays a significant role in negative regulation of cell proliferation, and RIZ1 expression is reduced in various cancers. RIZ2 was found to be upregulated in cancer cells, but its biological activity was not clear. It was mainly believed to be a non-functional counterpart of RIZ1, and it was hypothesized that RIZ2, acting as a negative regulator of RIZ1 function, mediates the proliferative effect of estrogen through regulation of survival and differentiation gene expression. J Cell Physiol 2012 March; 227(3):964-75. Additionally, RIZ2 being an N-terminal-truncated shorter transcript and translated product compared to RIZ1, the former presented less opportunity for manipulation independent of RIZ1.

The instant disclosure provide a method for targeting RIZ2, and compositions that inhibit RIZ2 independent of RIZ1.

Provided herein are therapeutic methods and compositions to specifically target and manipulate RIZ2. In some embodiments, the therapeutic composition comprises a pharmaceutical composition comprising a RIZ2-specific siRNA, for the treatment of a cell proliferative disorder. In some embodiments, the cell proliferative disorder is cancer.

Provided herein is a method of treating cancer, comprising administering to a subject in need thereof a pharmaceutical composition described in any of the sections above. As a result of the disclosed study, it is now understood that the RIZ2 provides a highly promising therapeutic target and not only can RIZ2 be selectively downregulated in cancer cells, RIZ1 can be upregulated by inhibiting RIZ2. It is also understood that RIZ/RIZ2 imbalance, e.g., downregulation of RIZ1 and upregulation of RIZ2 is a significantly upstream nodal regulation of the cell cycle, that is common in many cancers, and therefore the therapy is applicable to a wide variety of cancers. For instance, in some embodiments, the method and compositions described herein are therapeutically applicable to solid cancers. In some embodiments, the method and compositions described herein are therapeutically applicable to liquid cancers. In some embodiments, the cancer is a cancer of breast, colon, endometrial, esophageal, gastric, glioma, kidney, liver, lung, lymphoma, melanoma, meningioma, myeloma, nasopharyngeal, neuroblastoma, ovarian, pancreatic, parathyroid, pituitary, prostate, thyroid, or uterine tissue.

In some embodiments, the cancer is a solid tumor. In some embodiments, the cancer is a lung cancer. It is contemplated that any cancer may be treated using the methods and compositions described herein. Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

In other embodiments, the RIZ2 inhibitor, as described above and herein, is used to treat a hyperproliferative disorder, e.g., a hyperplasia, a cancer or hyperproliferative connective tissue disorder (e.g., a hyperproliferative fibrotic disease). In some embodiments, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the eye, lung, liver, heart, kidney, pancreas, skin and other organs). In other embodiments, the disorder is chosen from liver cirrhosis or tuberculosis. In other embodiments, the disorder is leprosy.

In some embodiments, the RIZ2 inhibitor is specifically designed to target and treat lung cancer. In some embodiments, the RIZ2 inhibitor is formulated for aerosol delivery into the lung. In some embodiments, the RIZ2 inhibitor is formulated for systemic delivery via intravenous injection.

In one embodiments, the RIZ2 inhibitor comprising the exemplary anti-RIZ2 siRNA described herein is designed for treating lung cancer. An exemplary anti-RIZ2 siRNA described herein comprises a sense strand having a sequence of SEQ ID NO: 6, or a sequence that is at least 95% identical to SEQ ID NO: 6, and an anti-sense strand having a sequence of SEQ ID NO: 11, or a sequence that is at least 95% identical to SEQ ID NO: 11.

In some embodiments, the exemplary anti-RIZ2 siRNA described herein exhibit no toxic effect on living cell, or experimental animals.

In some embodiments, the therapeutic composition comprising the RIZ2 inhibitor may be co-administered with a chemotherapeutic agent. The chemotherapeutic can be cyclophosphamide, doxorubicin, vincristine, prednisone, or rituximab, or a combination thereof. Other chemotherapeutics include obinutuzumab, bendamustine, chlorambucil, cyclophosphamide, ibrutinib, methotrexate, cytarabine, dexamethasone, cisplatin, bortezomib, fludarabine, idelalisib, acalabrutinib, lenalidomide, venetoclax, cyclophosphamide, ifosfamide, etoposide, pentostatin, melphalan, carfilzomib, ixazomib, panobinostat, daratumumab, elotuzumab, thalidomide, lenalidomide, or pomalidomide, or a combination thereof. “Co-administered” refers to administering two or more therapeutic agents or pharmaceutical compositions during a course of treatment. Such co-administration can be simultaneous administration or sequential administration. Sequential administration of a later-administered therapeutic agent or pharmaceutical composition can occur at any time during the course of treatment using the RIZ2 inhibitor.

In some embodiments, for example, a patient may be considered suitable for administering a dose regimen of a chemotherapeutic, e.g. a anti-PD1/PD-L1 therapy, and a dose regimen of a therapeutic described in the disclosure herein, comprising an anti-PRDM siRNA comprised in a nanoparticle and a cancer cell targeting aptamer, AS1411. In some embodiments, the anti-PRDM siRNA is an anti-RIZ2 siRNA. In some embodiments, the chemotherapeutic is a check point inhibitor. For example, in some embodiments, the checkpoint inhibitor is a PD1 blocker, e.g., pembrolizumab. For example, in some embodiments, the checkpoint inhibitor is a PD-L1 blocker, e.g., nivolumab. In some embodiments, the checkpoint is inhibitor ipilimumab.

In some embodiments, it is contemplated that treatment of anti-RIZ2 siRNA may sensitize cancer cells to a PD1/PD-L1 checkpoint inhibitor therapy. Based on the study disclosed herein, where the data indicates an increase in PD-L1 expression in lung cancer cell line upon treatment with an exemplary siRNA described herein, it follows that pretreatment with the siRNA followed by a checkpoint inhibitor regime (e.g., Keytruda) could result in increased efficacy of the drug. In some embodiments, pretreatment of a cancer or a tumor, e.g., NSCLC cell with anti-RIZ2 siRNA potentiates the latter treatment with the checkpoint inhibitor.

In some embodiments, the RIZ2 inhibitor siRNA may be co-administered with cisplatin.

In some embodiments, use of an siRNA described here (e.g., anti-RIZ2 siRNA comprising SEQ ID NO: 6 and SEQ ID NO: 11), with a drug as described above may provide advantage in that a lower concentration or dose of the drug can be sufficient for achieving the therapeutic effect, thereby reducing the drug related toxic effects in the subject with cancer.

In some embodiments, the RIZ2 inhibitor siRNA may be co-administered with an activator of EGR1 gene or gene cluster. In accordance to data provided herein, one or more genes that are significantly downregulated include the EGR1 gene. Accordingly, an agent that upregulates EGR1 could play an important role in augmenting the therapeutic effect of the siRNA on a cancer, when used in a combination therapy.

In some embodiments, is contemplated that treatment of anti-RIZ2 siRNA may sensitize cancer cells to an inhibitor of WNT signaling. Based on the study disclosed herein, where the data indicates the anti-proliferative action of an exemplary siRNA is dependent on inhibition of WNT signalling. Accordingly, it follows that a combination therapy of an anti-RIZ2 siRNA (e.g., ARIZ-047) together with a WNT inhibitor would improve efficacy of the siRNA.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

In some embodiments, the pharmaceutical composition comprising RIZ2 inhibitor may be administered once every day, once every 2 days, or 3 days, or 4 days or 5 days or 6 days or 7 days or 8 days or 9 days or 10 days or 11 days or 12 days or 13 days or 14 days. In some embodiments, the pharmaceutical composition comprising RIZ2 inhibitor may be administered once every 15 days. In some embodiments, the pharmaceutical composition comprising RIZ2 inhibitor may be administered once every 20 days. In some embodiments, the pharmaceutical composition comprising RIZ2 inhibitor may be administered once every month. In some embodiments, the pharmaceutical composition comprising RIZ2 inhibitor may be administered once every 2 months, or 3 months, or 4 months, or 5 months or 6 months. In some embodiments, the dosing schedule can be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art. The therapeutic formulation can be in unit dosage form. Such formulations include tablets, pills, capsules, powders, granules, solutions or suspensions in water or non-aqueous media, or suppositories.

In one aspect, provided herein is a method of patient selection that selecting a patient as a subject in need for administering a composition comprising a RIZ2 inhibitor, the method comprising: (i) detecting an elevated RIZ1 methylation level in a biological sample of the subject compared to a control sample or control value; or, (ii) detecting a downregulation of RIZ1 mRNA levels compared to a control sample or control value; wherein an at least 1.1-fold elevation of methylated RIZ1, or, at least 2-fold downregulation of RIZ1 mRNA levels in the biological sample of the subject compared to the control sample or value determines the subject to be in need for administering the composition comprising a RIZ2 inhibitor, and wherein the control sample is a sample from a clinically healthy individual, a control value is an average value of RIZ1 methylation levels from two or more clinically healthy individual samples.

In some embodiments, the RIZ2 inhibitor reduces hypermethylation of RIZ1 gene.

### Kits

The invention also provides kits comprising a RIZ2 inhibitor. Some aspects of this disclosure provide kits for the treatment of cancer, or a hyperproliferative disorder. In some embodiments, the kit is a neoplasia treatment kit.

In some embodiments, the kit may comprise a therapeutic composition comprising a RIZ2 inhibitor as described in the disclosure, in a properly labeled bottle with indicated concentration and directions for use, preserved in a temperature and environmental conditions that ensure safety, and efficacy of the therapeutic active ingredient.

In some embodiments, the kit may comprise a detector set for detecting RIZ1 and RIZ2 mRNA in a biological sample. The detector set may comprise a set of primers, e.g., a set of forward primers, and reverse primers for detecting RIZ1 levels, and a set of forward primers, and reverse primers for detecting RIZ2 levels. In some embodiments, RIZ 1 level is measured in sputum samples, and serve as biomarker for neoplasia. In some embodiments, the kit comprises one or more components for detecting RIZ1 levels and RIZ2 levels (e.g., mRNA, protein etc.) in a biological sample from a human. In some embodiments the kit may comprises a diagnosis unit for detecting RIZ1 methylation. In some embodiments, RIZ1 methylation can be detected using methylation based sequencing. In some embodiments, RIZ1 methylation may be detected by using one or more methylation based primers. The kit may comprise one or more vials comprising a primer, a reagent, an enzyme, a buffer etc. Each vial is labeled with component, concentration. The kit comprises at least one written instruction sheet for use of the kit.

The neoplasia treatment kit comprises written instructions for using the modified immune cells in the treatment of the neoplasia.

The detection kit comprises written instructions on the components included safety parameters and directions for use.

In some embodiments, a single kit may comprise components of a therapeutic kit and a detection kit.

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The section headings used herein are for organizational purposes only and are not to be construed as limiting the subject matter described.

### INCORPORATION BY REFERENCE

All publications and patents mentioned herein are hereby incorporated by reference in their entirety as if each individual publication or patent was specifically and individually indicated to be incorporated by reference.

### EXAMPLES

The following examples are included for illustrative purposes only and are not intended to limit the scope of the invention.

### Example 1: Targeted Drug Delivery Design

Two exemplary nanoparticle composition designs are shown here for a targeted drug delivery system for cancer treatment with the siRNA disclosed herein. In some embodiments, a targeted siRNA nanoparticle is designed by conjugating a DNA Aptamer (e.g., AS1411) directly to the siRNA (optionally via a linker), graphically represented in FIG. 1A (left). The linker is a PEG-2000 molecule or any other suitable linker. In another embodiment, the aptamer (e.g., AS1411) is conjugated to PEG 2000, and combined with 8 fold the amount of PEG 2000-conjugated exemplary siRNA (e.g., anti-RIZ2 siRNA, comprising a sense strand (SEQ ID NO:6) and an antisense strand (SEQ ID NO: 11)) (FIG. 1A, right). These were then mixed with the ingredients necessary to add the nanoparticle composition coating. When this nanoparticle is assembled, the size is around 150 nanometers in size (FIG. 1i). Based on the size and charge, the majority of the aptamer (e.g., AS1411) is displayed on the outside on the nanoparticle composition coating. In this example, the nanoparticle is a calcium phosphate nanoparticle.

### Example 2. Generation of Mouse Xenograft Model of Lung Cancer

In this example, mouse xenograft lung cancer model for testing the siRNA was utilized. A549 cells were engrafted into nude mice which developed A549 lung cell tumor in a few days in these mice. An exemplary siRNA having a sense strand sequence of SEQ ID NO: 6 and an antisense strand sequence of SEQ ID NO: 11 was delivered in a targeted nanoparticle composition comprising PEG-conjugated anti-nucleolin aptamer AS1411 as shown in FIG. 1A (right) and FIG. 1C, in which the siRNA was dosed at 1 mg/kg. In parallel assay sets, the same siRNA sense and antisense pair was delivered in nanoparticle compositions lacking the targeting aptamer (no targeting control).

### Example 3. Testing Efficacy In Vivo of siRNA Using Targeted siRNA Delivery

A targeted drug delivery system for cancer treatment, comprising a calcium phosphate nanoparticle displaying a targeting ligand was used in this exemplary study to deliver a PRDM2 siRNA payload specifically to cancer cells in an animal model.

Female athymic nude mice were used to prepare lung adenocarcinoma tumor model with A549 cells. An injectable formulation of calcium phosphate nanoparticle compositions with either AS1411-PEG plus PEG-ARIZ-047 or PEG-ARIZ-047 siRNA were injected intravenously into mice bearing A549 lung cancer tumor xenografts. Dosing was performed on days 0, 3, 5, 8, 10, 12, 15, 18, 22. The mice were monitored for tumor size for the effect of AS1411/siRNA. At day 31 mice were sacrificed and tumor was excised and evaluated. Blood and other tissues were harvested for mRNA levels for RNA-SEQ analysis for gene expression compared to suitable controls. Vehicle control was 5% dextrose in water. For positive control, carboplatin was used at 30 mg/kg concentration, dosed on the same days.

As shown in the data (FIG. 1C), use of the targeting agent AS1411 greatly enhanced suppression of tumor growth compared to siRNA in a non-targeted nanoparticle composition. Surprisingly, AS1411 targeted Ariz-047 was even more effective than carboplatin, and over the period of duration of the study of one month, the tumor progression was almost negligible in the targeted nanoparticle composition treated set compared to 7-fold increase in the non-treated vehicle control.

As shown in the data (FIG. 1D) use of the targeting agent AS1411 greatly enhanced intracellular knockdown of the mRNA for the target RIZ2 compared to siRNA in a non-targeted nanoparticle composition.

### Example 4: Changes in Gene Expression Upon ARIZ-047 siRNA Treatment

In this example, the effect of PRDM2 siRNA (siRNA sense strand: SEQ ID NO: 6, antisense strand SEQ ID NO: 11) was tested in a non-small cell lung cancer cell line A549.

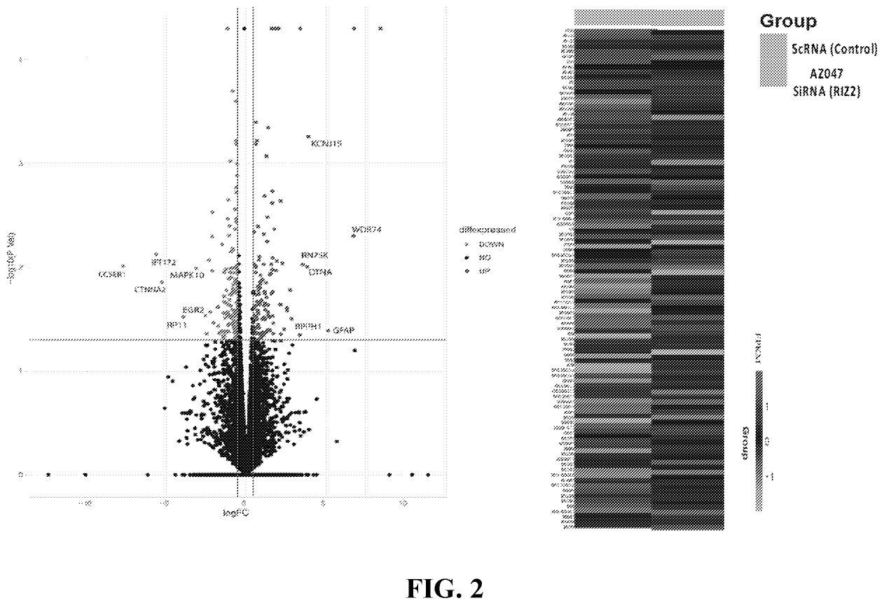

RNA-Seq gene expression analysis was performed in these cells to understand the mechanism of action of the ARIZ-047 siRNA in lung cancer cells. It was found that ARIZ-047 changes RNA expression landscape of A549 lung cancer cell line. Data shown in FIGS. 2, 3, 4A-4C illustrate that RNA-Seq identified key pro-cancer signaling pathways that are affected by ARIZ-047 treatment. FIG. 2 shows the dispersion of RNA Seq gene expression data in the presence of the siRNA versus control (scramble) in a volcano plot (left panel), which is also demonstrated in the plot in the right panel (FIG. 2). Genes with upregulated expression are on the right side of the volcano plot, and genes with downregulated expression are on the left side of the plot. A total of 61 and 50 differentially expressed genes were down and upregulated, respectively. These results suggest that ARIZ047 treatment elicits significant changes in gene expression in A549 cells, providing insight into the molecular mechanisms underlying its therapeutic effects in lung cancer.

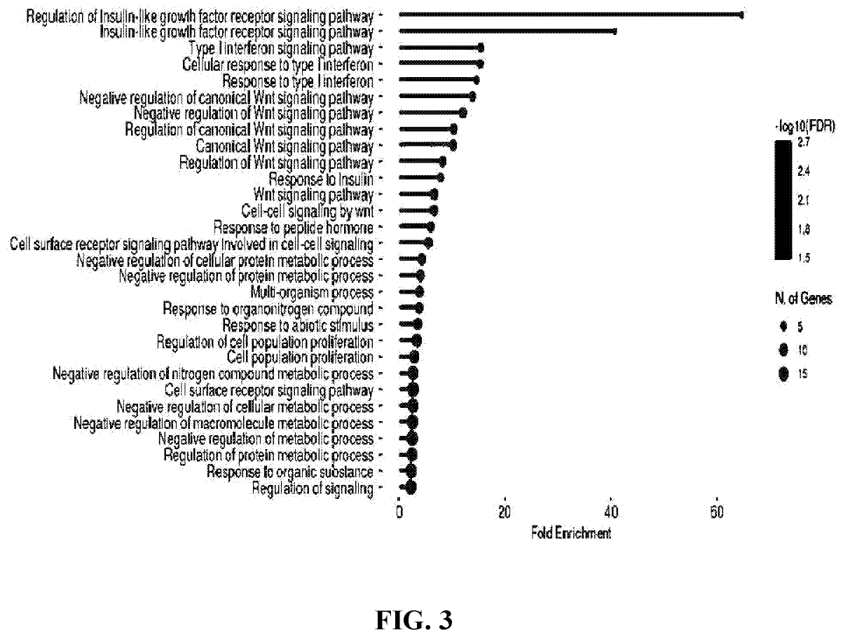

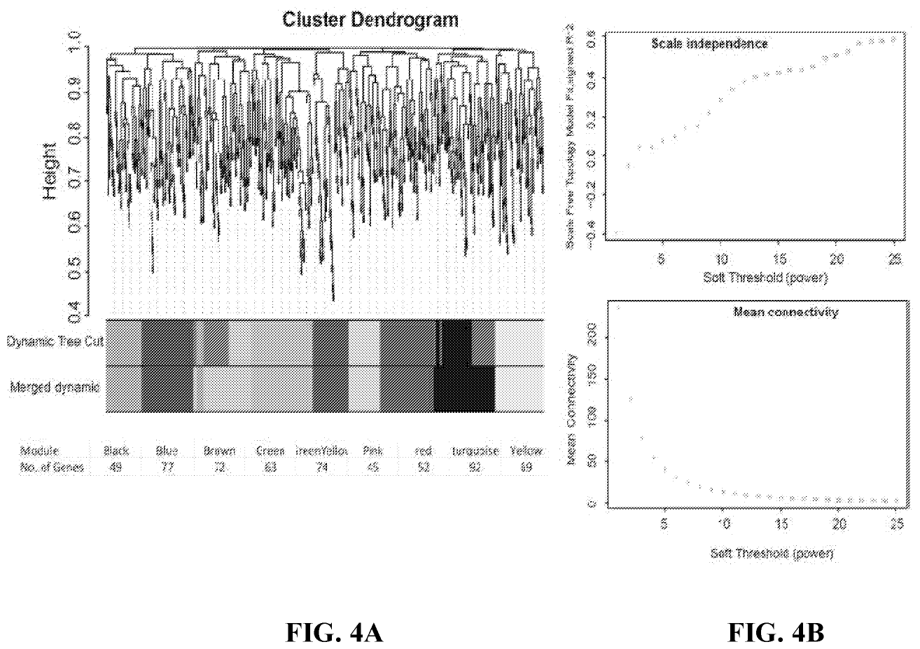

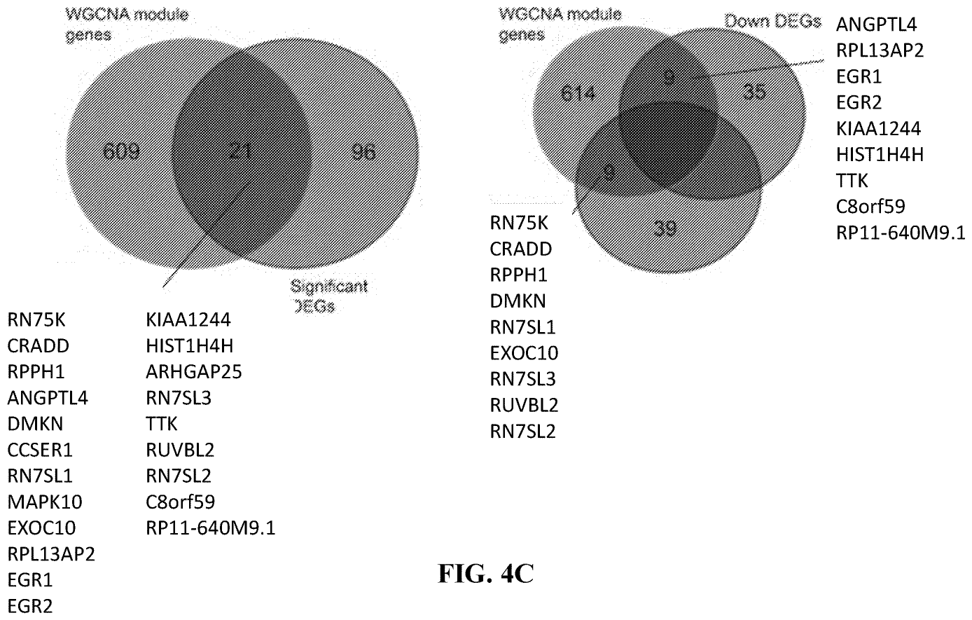

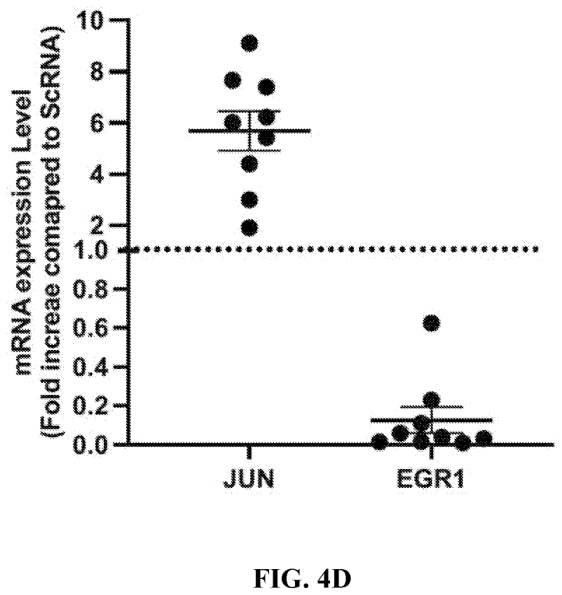

Gene ontology (GO) term enrichment analysis revealed overrepresented GO terms for downrugulated and upregulated differentially expressed genes (DEGs) in ARIZ047 siRNA-treated cells (FIG. 3). The gene expression profile showed that insulin like growth factor receptor family of genes, IFN responsive family of early response genes were most implicated in response to the siRNA treatment. Negative regulators of the WNT signaling pathway clusters were also highly upregulated (FIG. 4). EGR1 was indicated as a major driver gene for these pro-cancer signaling pathways in A549 NSCLC cells, as was CTGF, JUN, and FOS (FIG. 4.)

These data indicated that inhibition of WNT signaling could play a major role in the siRNA mediated tumor cell anti-proliferative activity. To test this, tumor cells were treated with an activator of WNT signaling pathway, R-spondin. Briefly, A549 cells were transfected with anti-RIZ2 siRNA or control ScRNA. After transfection A549 cells were cultured on Matrigel with growth factors to form 3D spheroids. Three transfection experiments were performed in duplicate. In one representative example, replicates of one set of ARIZ-047 transfected cells were cultured in presence of WNT signaling agonist R-spondin. Micrographic images were acquired after 6 days. R-spondin is used at 10 ng/ml concentration. 8 h after transfection in 2D culture, cells were embedded in matrigel supplemented with 100 ul of growth medium that contains 100 ng/ml R-spondin. Every two days medium was changed to fresh medium that contains 100 ng/ml R-spondin.

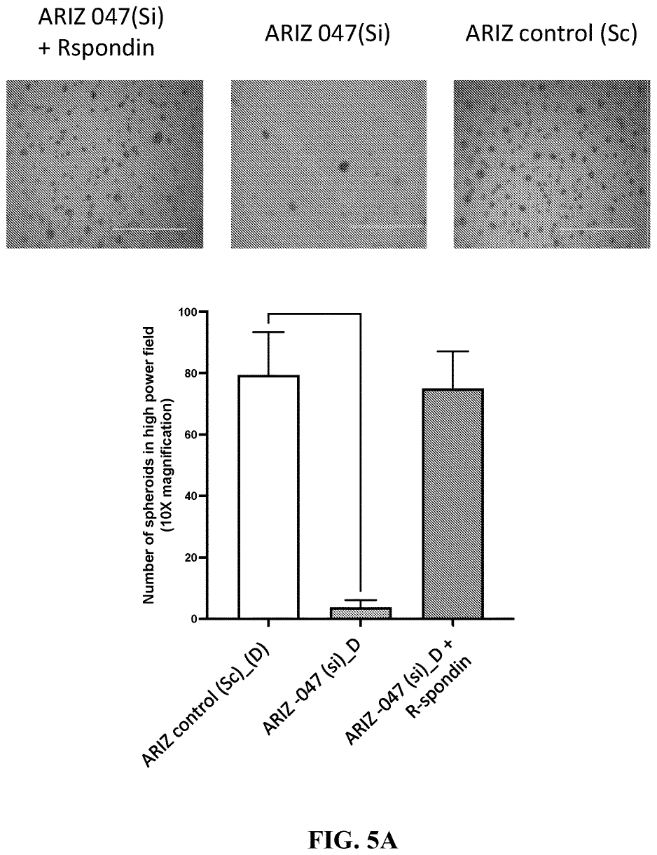

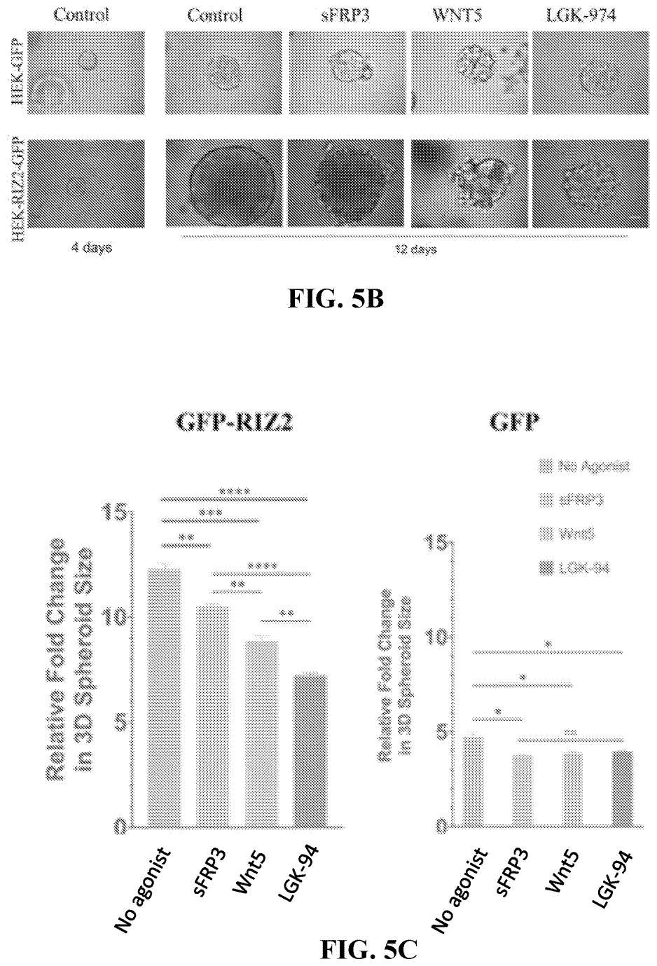

Microscopic images obtained over 6 days revealed that A549 cells treated with ARIZ047 failed to form 3D organoids compared to those treated with control ScRNA (FIG. 5A). Constitutive activation of WNT signaling in anti-RIZ2 siRNA (ARIZ-047) treated A549 3D spheroids reverses the cell killing effect of the siRNA, demonstrating that a crucial mechanism of action for cell killing by the siRNA treatment was through inhibition of the WNT signaling pathway. This is illustrated in the data shown in FIG. 5A. The graph presented in the right panel of FIG. 5 indicates the proliferation of the tumor cell A549 spheroids in the presence of the siRNA, compared to the scramble control. Dramatic reduction of the number of spheroids (about 70-80 fold) was observed in siRNA treated set over the scramble control set. R-espondin treatment led to activation of the WNT signaling, which restored the proliferation of the spheroids. Based on this data, it is inferred that the anti-proliferative action of the siRNA that inhibits RIZ2 is largely dependent on the inhibition of the WNT signaling in the cancer cells and may point to a possible utility for a combination therapy of ARIZ-047 and a WNT inhibitor.

The relationship between RIZ2 and the WNT signaling pathway was further investigated by assessing the effect of Wnt signaling pathway agonists sFRP3, WNT5, and LGK-974 on 3D spheroids overexpressing the RIZ2 protein derived from BEK cells (FIG. 5B, lower panel) compared to control spheroids lacking RIZ2 (FIG. 5B, upper panel). Notably after four days, Phase contrast microscopic images obtained at 4 days showed the formation of well-defined roundish and well differentiated 3D spheroids in both GFP only and RIZ2-GFP overexpressing HEK-293 cells cultured in Matrigel. Subsequently, changes in the size and morphology of the spheroids overexpressing RIZ2-GFP and GFP only were monitored over an additional 12-day period in the presence of negative regulators of Wnt signaling pathway. Interestingly, an increase of about 12-fold in the size of pEGFP RIZ2-derived organoids was observed after 12 days.

After 12 days of culturing, spheroids lacking RIZ2 displayed a moderate increase in size, whereas spheroids expressing RIZ2 exhibited a significant increase in size. Intriguingly, when RIZ2-overexpressing spheroids were subjected to the treatment with Wnt signaling agonist, a substantial reduction in size was observed compared to the untreated spheroids. Among the Wnt-signaling agonists tested, LGK-974 was able to significantly reduce the size of the RIZ2-GFP overexpressing spheroids. In contrast, the spheroids lacking RIZ2 did not exhibit any alterations in size when exposed to sFRP3, WNT5, and LGK-974 (FIG. 5C). These findings suggest that LGK-974 is more effective in presence of RIZ2 which plays a pivotal role in modulating WNT signaling and contributes to tumor progression in Lung cancer. These data further support a possible utility for a combination therapy of ARIZ-047 and a WNT inhibitor.

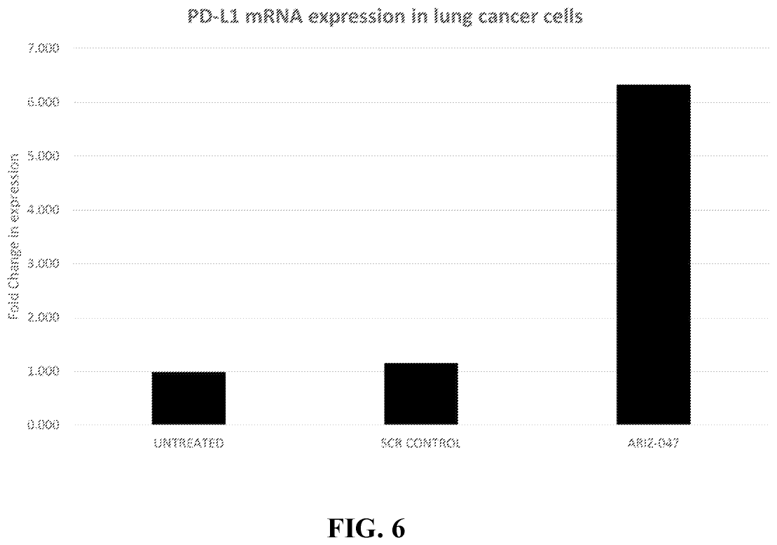

Anti-RIZ2 siRNA treated cells also show upregulation of PD-L1 in cancer cells, as illustrated in FIG. 6. Compared to untreated cells or scrambled control, siRNA treated cells showed an about 6-fold increase in PD-L1 expression. This indicates that treatment with anti-RIZ2 siRNA could further potentiate a checkpoint inhibitor therapy in cancer, such as pembrolizumab (Keytruda).

### Example 5: Nanoparticle Formation

This example describes a 3-step process of forming nanoparticles disclosed herein.

Step 1: Aptamer PEGylation. This step produces a DNA aptamer (e.g., AS1411) with PEG attached to the 5′ end of the DNA aptamer.

Solutions of 0.431M methoxy-PEG-NH2 2000 (Laysan Bio) in 0.172M Imidazole HCL (Sigma) pH6, 852 mM EDC (N-(3-Dimethylaminopropyl)-N′-ethylcarbodiimide hydrochloride: Sigma) in 1.26 mL 1M MES (2-(N-Morpholino)ethanesulfonic acid: Sigma) buffer pH4, and 1 mM DNA aptamer (e.g., AS1411) in water were prepared. In each tube, 50 uL aptamer, 460 uL nuclease free water, 105 uL MES/EDC, and 435 uL PEG/Imidazole were mixed and incubated at 50° C. under nitrogen gas overnight while stirring.

The reaction mixture was slowly cooled and transferred to a 4 ml 3K MWCO filter, that was pre-washed 4 times once with 0.05M NaOH then 3 times with nuclease free water at 7,500 g. Nuclease free water was added to the solution (1.5 mL reaction mix to 2.5 mL water). The aptamer solution was centrifuged at 7500 g until the volume of water added is in the flow through. This step was repeated 4 more times, increasing the water added and thus collected by 0.5 mL each time (the last two cycles use the same volume). The mixture was collected and resuspended to the desired concentration. An EtBr agarose gel was run to determine the PEGylation percent by image densitometry.

Step 2: siRNA PEGylation. This step produces an siRNA with PEG attached to the 5′ end of the sense strand.

0.431M methoxy-PEG-NH2 2000 (Laysan Bio) in 0.172M Imidazole HCL (Sigma) pH6, 852 mM EDC (N-(3-Dimethylaminopropyl)-N′-ethylcarbodiimide hydrochloride: Sigma) in 1.26 mL 1M MES (2-(N-Morpholino)ethanesulfonic acid: Sigma) buffer pH4, and 1 mM of an siRNA sense strand were prepared. To make PEGylated sense strands, 50 uL siRNA sense strand (e.g., SEQ ID NO: 6), 460 uL nuclease free water, 105 uL MES/EDC, and 435 uL PEG/Imidazole were mixed and incubated at 50 C under nitrogen gas overnight while stirring.

To anneal the corresponding siRNA antisense strand to the pegylated sense strand, the reaction mixture with the siRNA sense strand was slowly cooled and 50 uL 1 mM of a corresponding siRNA antisense strand (e.g., SEQ ID NO: 11) was added, solution was vortexed, and incubated at 70° C. for 20 minutes under nitrogen and stirring. The reaction mixture was slow cooled over 2 hours. The reaction mixture was added to a 50 mL tube, then 1 mL of 5M sodium acetate and 13 mL ethanol were added. The mixture was vortexed and incubated at −80 C overnight.

Tubes were vortexed then centrifuged at 15,000 g at 4 C for 90 minutes. The supernatant was removed and saved. 4 mL-20 C 75% ethanol was added to the tube and incubated for 90 minutes at −80 C, then centrifuged at 15,000 g at 4 C for 45 minutes. The supernatant was removed and saved; tubes were covered with a Kimwipe and left inverted at 4 C to dry overnight.

Then tubes were brought to room temperature and samples resuspended and vortexed with 100 uL nuclease free water. An EtBr agarose gel was run to determine the PEGylation percent. Equal volumes of 1 mM non-PEGylated sense and antisense RNA were mixed heated at 70 C for 20 minutes and slow cooled. Annealed non-PEG siRNA was added to PEG-siRNA to achieve 12.5% PEGylation.

Step 3: Calcium Phosphate nanoparticle assembly

25 mM Calcium Chloride (CaCl₂)) (Sigma) and 25 mM Sodium Phosphate (Na2HPO4) (Sigma) were made to a pH of 10. PEG-siRNA (12.5% PEGylated), CaCl₂), and PEG-aptamer (75% PEGylated) were mixed in solution to get a concentration of 200 uM (12.5% PEGylated siRNA), 5 mM CaCl₂), and 25 uM PEG-aptamer. Na2HpO4 was diluted to get a 4.05 mM concentration based on the total desired solution volume and was added last. Solutions were vortexed between additions. The solutions were prepared and incubated for 2 days at RT. After incubation, nanoparticle size was examined by Dynamic Light Scattering. A 500 ul 100K MWCO centrifugal filter was washed 4 times, once with 0.05M NaOH then 3 times with nuclease free water at 3,000 g until most of the solution was passed through the filter. Nanoparticle solution was centrifuged with the pre-washed 100K MWCO filter at 3,000 g until 90% of the solution volume has passed through the filter. The sample was collected and 25 mM CaCl₂) pH10 was added to increase the concentration of CaCl₂ by 2 mM, then vortexed. The solution was incubated at 37 C for 2 hours, slow cooled, vortexed, and incubated overnight at RT. The solution pH was measured and nanoparticle size was examined by Dynamic Light Scattering. A syringe filter was pre-washed with 0.5 ml water, then sample was passed through the filter. Samples were collected for DLS and siRNA quantification.

The present invention is not intended to be limited in scope to the particular disclosed embodiments, which are provided by way of example to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-76. (canceled)

77. A composition for treating a cancer in a subject, comprising:
a therapeutically effective amount of a PRDM inhibitor; and
a cancer cell targeting moiety that binds specifically to a nuclear protein that is expressed on a cancer cell surface.

78. The composition of claim 77, wherein the PRDM is a PRDM2, and wherein the PRDM2 is RIZ2.

79. The composition of claim 77, wherein the PRDM inhibitor is selected from a group consisting of small molecules, peptides, antibodies, and nucleic acid inhibitors.

80. The composition of claim 77, wherein the cancer cell targeting moiety comprises a DNA aptamer that specifically binds to a nuclear protein expressed on the surface of the cancer cell, and wherein the DNA aptamer is pegylated.

81. The composition of claim 80, wherein the nuclear protein expressed on the surface of the cancer cell is nucleolin, and wherein the DNA aptamer specifically binds to the nucleolin.

82. The composition of claim 77, wherein the cancer cell targeting moiety comprises a nucleic acid having a sequence with at least 85% sequence identity to the sequence of SEQ ID NO: 21.

83. The composition of claim 77, wherein the cancer cell targeting moiety comprises a nucleic acid having a sequence set forth in SEQ ID NO: 21 or a sequence that has less than 3 nucleotides differing from that of SEQ ID NO: 21.

84. The composition of claim 77, further comprising a nanoparticle delivery vehicle, comprising: (a) a therapeutically effective amount of an anti-PRDM2 siRNA molecule comprising a sense strand and an antisense strand, wherein the sense strand and the antisense strand are selected from Table 2; (b) a DNA aptamer that binds to a nuclear protein expressed on the surface of a cancer cell having a sequence that is at least 85% sequence identity to the sequence of SEQ ID NO: 21.

85. The composition of claim 84, wherein the sense strand has a sequence that is at least 95% identical to SEQ ID NO: 6, and the antisense strand has a sequence that is at least 95% identical to SEQ ID NO: 11.

86. The composition of claim 84, wherein the anti-PRDM2 siRNA molecule comprises a double stranded RNA comprising a sense strand and an antisense strand, each strand having 10-21 nucleotides, wherein the sense strand and the antisense strand hybridize with each other to form at least a region of double strands, and wherein each of the sense strand and the antisense strand has at least one nucleotide overhang at the 5′ or the 3′ end.

87. The composition of claim 84, wherein the anti-PRDM2 siRNA molecule comprises at least 10 contiguous nucleotides that are homologous to 10 contiguous nucleotides of the human PRDM2/RIZ2 gene.

88. The composition of claim 84, wherein the anti-PRDM2 siRNA molecule is covalently linked with a poly-ethylene glycol (PEG) moiety (pegylated siRNA).

89. The composition of claim 84, wherein the anti-PRDM2 siRNA molecule comprises one or more modified nucleotide bases.

90. A pharmaceutical composition comprising:
(i) a calcium phosphate nanoparticle delivery vehicle, comprising:
(a) a therapeutically effective amount of an anti-PRDM2 siRNA comprising a sense strand having a sequence set forth in SEQ ID NO: 6; and an anti-sense strand having a sequence set forth in SEQ ID NO: 11; and
(b) a cancer cell targeting DNA aptamer, having a sequence of SEQ ID NO: 21, wherein the cancer cell targeting DNA aptamer binds to nucleolin on a cancer cell.

91. An injectable formulation for anti-cancer therapy, comprising:
(a) a double stranded siRNA having a sense strand comprising a sequence of SEQ ID NO: 6; and an antisense comprising a sequence of SEQ ID NO: 11;
(b) a calcium phosphate nanoparticle encapsulating the siRNA of (a);
(c) a cancer cell targeting moiety covalently associated with the siRNA of (a) or the nanoparticle of (b), having a nucleic acid sequence of SEQ ID NO: 21; and
(d) a pharmaceutically acceptable excipient.

92. A method of reducing cancer cell proliferation, comprising contacting the cancer cells with the composition of claim 77.

93. A method of treating lung cancer in a human subject, comprising administering to the subject a pharmaceutical composition comprising:
(a) a therapeutically effective amount of an siRNA comprising a sense strand having the sequence of SEQ ID NO: 6, and an antisense strand having the sequence of SEQ ID NO: 11;
(b) a calcium phosphate nanoparticle encapsulating the siRNA of (a);
(c) a cancer cell targeting moiety covalently associated with the siRNA of (a) or the nanoparticle of (b), wherein the targeting moiety is a nucleolin binding aptamer having a sequence of SEQ ID NO: 21; and
(d) a pharmaceutically acceptable excipient.

94. The method of claim 93, wherein the pharmaceutical composition is administered in combination with a second therapy.

95. The method of claim 94, wherein the second therapy is an anti-cancer therapy, selected from radiation therapy, chemotherapy, a therapy involving EGRI pathway activation, or a therapy involving inhibition of WNT signaling pathway.

96. The method of claim 94, wherein the second therapy is an anti-PD-1 or PD-L1 therapy.
